SEQUENCE LISTING (69 sequences)

SEQ ID NO: 1 - protein, 13 aa, Artificial Sequence
  Description of Artificial Sequence: Synthetic peptide
  DISULFID (2)..(12)
ICVVQDWGHHRCT

SEQ ID NO: 2 - protein, 42 aa, Artificial Sequence
  Description of Artificial Sequence: Synthetic polypeptide
  VARIANT (1)..(19): Any independently selected amino acid or amino acid analog
  misc_feature (1)..(19): This region may encompass 2 to 19 residues, wherein some residues may be absent
  VARIANT (22)..(22): Trp or an analog of Trp
  VARIANT (24)..(42): Any independently selected amino acid or amino acid analog
  misc_feature (24)..(42): This region may encompass 2 to 19 residues, wherein some residues may be absent
XXXXXXXXXXXXXXXXXXXQDXGXXXXXXXXXXXXXXXXXXX

SEQ ID NO: 3 - protein, 5 aa, Artificial Sequence
  Description of Artificial Sequence: Synthetic peptide
  VARIANT (1)..(1): Trp or an analog of Trp
  VARIANT (4)..(4): Trp or an analog of Trp
XQDXG

SEQ ID NO: 4 - protein, 6 aa, Artificial Sequence
  Description of Artificial Sequence: Synthetic peptide
  VARIANT (1)..(1): Trp or an analog of Trp
  VARIANT (4)..(4): Trp or an analog of Trp
  VARIANT (6)..(6): His, Ala, analogs of Ala, Phe or Trp
XQDXGX

SEQ ID NO: 5 - protein, 13 aa, Artificial Sequence
  Description of Artificial Sequence: Synthetic peptide
  VARIANT (1)..(3): Any independently selected amino acid or amino acid analog
  VARIANT (4)..(4): Trp or analog of Trp
  VARIANT (7)..(7): Trp or analog of Trp
  VARIANT (9)..(13): Any independently selected amino acid or amino acid analog
XXXXQDXGXXXXX

SEQ ID NO: 6 - protein, 16 aa, Artificial Sequence
  Description of Artificial Sequence: Synthetic peptide
  MISC_FEATURE: see specification as filed for detailed description of substitutions and preferred embodiments
  VARIANT (1)..(2): Ile, Val, Leu, B1-Ile, B1-Val, B1-Leu or a dipeptide comprising Gly-Ile or B1-Gly-Ile, wherein B1 represents a first blocking moiety
  misc_feature (1)..(2): This region may encompass 1 to 2 residues, wherein some positions may be absent
  DISULFID (3)..(13)
  VARIANT (5)..(5): Independently selected from Trp or analogs of Trp
  VARIANT (8)..(8): Independently selected from Trp or analogs of Trp
  VARIANT (10)..(10): His, Ala or an analog of Ala, Phe, Trp or an analog of Trp
  VARIANT (14)..(16): L-Thr, D-Thr, Ile, Val, Gly, a dipeptide selected from Thr-Ala and Thr-Asn, or a tripeptide comprising Thr-Ala-Asn, wherein a C-terminal -OH of any of the L-Thr, D-Thr, Ile, Val, Gly, Ala or Asn is optionally replaced by a second blocking moiety
  misc_feature (14)..(16): This region may encompass 1 to 3 residues, wherein some positions may be absent
XXCVXQDXGXHRCXXX

SEQ ID NO: 7 - protein, 16 aa, Artificial Sequence
  Description of Artificial Sequence: Synthetic peptide
  misc_feature: see specification as filed for detailed description of substitutions and preferred embodiments
  VARIANT (1)..(2): Ile, Val, Leu, Ac-Ile, Ac-Val, Ac-Leu or a dipeptide comprising Gly-Ile or Ac-Gly-Ile
  misc_feature (1)..(2): This region may encompass 1 to 2 residues, wherein some positions may be absent
  DISULFID (3)..(13)
  VARIANT (5)..(5): Independently selected from Trp or analogs of Trp
  VARIANT (8)..(8): Independently selected from Trp or analogs of Trp
  VARIANT (10)..(10): His, Ala or an analog of Ala, Phe, Trp or an analog of Trp
  VARIANT (14)..(16): L-Thr, D-Thr, Ile, Val, Gly, a dipeptide selected from Thr-Ala or Thr-Asn, or a tripeptide comprising Thr-Ala-Asn, wherein a c-term -OH of any of L-Thr, D-Thr, Ile, Val, Gly, Ala, or Asn is optionally replaced by -NH2
  misc_feature (14)..(16): This region may encompass 1 to 3 residues, wherein some positions may be absent
XXCVXQDXGXHRCXXX

SEQ ID NO: 8 - protein, 13 aa, Artificial Sequence
  Description of Artificial Sequence: Synthetic peptide
  MISC_FEATURE: C-terminal CONH2
  MOD_RES (1)..(1): N-terminal H
ICVVQDWGHHRCT

SEQ ID NO: 9 - protein, 13 aa, Artificial Sequence
  Description of Artificial Sequence: Synthetic peptide
  MOD_RES (1)..(1): N-terminal ACETYLATION
  MOD_RES (13)..(13): C-terminal CONH2
ICVVQDWGHHRCT

SEQ ID NO: 10 - protein, 13 aa, Artificial Sequence
  Description of Artificial Sequence: Synthetic peptide
  MOD_RES (1)..(1): N-terminal ACETYLATION
  MOD_RES (13)..(13): C-terminal CONH2
ICVYQDWGAHRCT

SEQ ID NO: 11 - protein, 13 aa, Artificial Sequence
  Description of Artificial Sequence: Synthetic peptide
  MOD_RES (1)..(1): N-terminal ACETYLATION
  MOD_RES (13)..(13): C-terminal COOH
ICVWQDWGAHRCT

SEQ ID NO: 12 - protein, 13 aa, Artificial Sequence
  Description of Artificial Sequence: Synthetic peptide
  MOD_RES (1)..(1): N-terminal ACETYLATION
  MOD_RES (13)..(13): C-terminal CONH2
ICVWQDWGAHRCT

SEQ ID NO: 13 - protein, 13 aa, Artificial Sequence
  Description of Artificial Sequence: Synthetic peptide
  MOD_RES (1)..(1): N-terminal ACETYLATION
  MOD_RES (13)..(13): d-Thr
  MOD_RES (13)..(13): C-terminal COOH
ICVWQDWGAHRCT

SEQ ID NO: 14 - protein, 13 aa, Artificial Sequence
  Description of Artificial Sequence: Synthetic peptide
  MOD_RES (1)..(1): N-terminal ACETYLATION
  MOD_RES (4)..(4): 2-napthylalanine
  MOD_RES (13)..(13): C-terminal CONH2
ICVAQDWGAHRCT

SEQ ID NO: 15 - protein, 13 aa, Artificial Sequence
  Description of Artificial Sequence: Synthetic peptide
  MOD_RES (1)..(1): N-terminal ACETYLATION
  MOD_RES (4)..(4): 2-naphthylalanine
  MOD_RES (13)..(13): C-terminal COOH
ICVAQDWGAHRCT

SEQ ID NO: 16 - protein, 13 aa, Artificial Sequence
  Description of Artificial Sequence: Synthetic peptide
  MOD_RES (1)..(1): N-terminal ACETYLATION
  MOD_RES (4)..(4): 1-naphthylalanine
  MOD_RES (13)..(13): C-terminal COOH
ICVAQDWGAHRCT

SEQ ID NO: 17 - protein, 13 aa, Artificial Sequence
  Description of Artificial Sequence: Synthetic peptide
  MOD_RES (1)..(1): N-terminal ACETYLATION
  MOD_RES (4)..(4): 2-indanylglycine carboxylic acid
  MOD_RES (13)..(13): C-terminal CONH2
ICVGQDWGAHRCT

SEQ ID NO: 18 - protein, 13 aa, Artificial Sequence
  Description of Artificial Sequence: Synthetic peptide
  MOD_RES (1)..(1): N-terminal ACETYLATION
  MOD_RES (4)..(4): 2-indanylglycine carboxylic acid
  MOD_RES (13)..(13): C-terminal COOH
ICVGQDWGAHRCT

SEQ ID NO: 19 - protein, 13 aa, Artificial Sequence
  Description of Artificial Sequence: Synthetic peptide
  MOD_RES (1)..(1): N-terminal ACETYLATOIN
  MOD_RES (4)..(4): dihydrotrpytophan
  MISC_FEATURE (13)..(13): C-terminal COOH
ICVWQDWGAHRCT

SEQ ID NO: 20 - protein, 13 aa, Artificial Sequence
  Description of Artificial Sequence: Synthetic peptide
  MOD_RES (1)..(1): N-terminal ACETYLATION
  MOD_RES (4)..(4): 4-benzoyl-L-phenylalanine
  MOD_RES (13)..(13): C-terminal COOH
ICVFQDWGAHRCT

SEQ ID NO: 21 - protein, 13 aa, Artificial Sequence
  Description of Artificial Sequence: Synthetic peptide
  MOD_RES (1)..(1): N-terminal ACETYLATION
  MOD_RES (4)..(4): p-benzoyl-L-phenylalanine
  MOD_RES (13)..(13): C-terminal CONH2
ICVFQDWGAHRCT

SEQ ID NO: 22 - protein, 13 aa, Artificial Sequence
  Description of Artificial Sequence: Synthetic peptide
  MOD_RES (1)..(1): N-terminal ACETYLATION
  MOD_RES (4)..(4): Bta
  MOD_RES (13)..(13): C-terminal COOH
ICVXQDWGAHRCT

SEQ ID NO: 23 - protein, 13 aa, Artificial Sequence
  Description of Artificial Sequence: Synthetic peptide
  MOD_RES (1)..(1): N-terminal ACETYLATION
  MOD_RES (4)..(4): Bta
  MOD_RES (13)..(13): C-terminal CONH2
ICVXQDWGAHRCT

SEQ ID NO: 24 - protein, 13 aa, Artificial Sequence
  Description of Artificial Sequence: Synthetic peptide
  MOD_RES (1)..(1): N-terminal ACETYLATION
  MOD_RES (9)..(9): 2-alpha-aminobutyric acid
  MOD_RES (13)..(13): C-terminal CONH2
ICVWQDWGXHRCT

SEQ ID NO: 25 - protein, 16 aa, Artificial Sequence
  Description of Artificial Sequence: Synthetic peptide
  MOD_RES (1)..(1): N-terminal H
  MOD_RES (16)..(16): C-terminal COOH
GICVWQDWGAHRCTAN

SEQ ID NO: 26 - protein, 13 aa, Artificial Sequence
  Description of Artificial Sequence: Synthetic peptide
  MOD_RES (1)..(1): N-terminal ACETYLATION
  MOD_RES (4)..(4): 5-fluoro-L-tryptophan
  MOD_RES (13)..(13): C-terminal CONH2
ICVWQDWGAHRCT

SEQ ID NO: 27 - protein, 13 aa, Artificial Sequence
  Description of Artificial Sequence: Synthetic peptide
  MOD_RES (1)..(1): N-terminal ACETYLATION
  MOD_RES (4)..(4): 5-methyl-L-tryptophan
  MOD_RES (13)..(13): C-terminal CONH2
ICVWQDWGAHRCT

SEQ ID NO: 28 - protein, 13 aa, Artificial Sequence
  Description of Artificial Sequence: Synthetic peptide
  MOD_RES (1)..(1): N-terminal Ac
  MOD_RES (4)..(4): 1-methyl-L-tryptophan
  MOD_RES (13)..(13): C-terminal CONH2
ICVWQDWGAHRCT

SEQ ID NO: 29 - protein, 13 aa, Artificial Sequence
  Description of Artificial Sequence: Synthetic peptide
  MOD_RES (1)..(1): N-terminal ACETYLATION
  MOD_RES (7)..(7): 5-fluoro-L-tryptophan
  MOD_RES (13)..(13): C-terminal CONH2
ICVWQDWGAHRCT

SEQ ID NO: 30 - protein, 13 aa, Artificial Sequence
  Description of Artificial Sequence: Synthetic peptide
  MOD_RES (1)..(1): N-terminal ACETYLATION
  MOD_RES (4)..(4): 5-fluoro-L-tryptophan
  MOD_RES (7)..(7): 5-fluoro-L-tryptophan
  MOD_RES (13)..(13): C-terminal CONH2
ICVWQDWGAHRCT

SEQ ID NO: 31 - protein, 13 aa, Artificial Sequence
  Description of Artificial Sequence: Synthetic peptide
  MOD_RES (1)..(1): N-terminal ACETYLATION
  MOD_RES (4)..(4): 5-methyl-L-tryptophan
  MOD_RES (7)..(7): 5-fluoro-L-tryptophan
  MOD_RES (13)..(13): C-terminal CONH2
ICVWQDWGAHRCT

SEQ ID NO: 32 - protein, 13 aa, Artificial Sequence
  Description of Artificial Sequence: Synthetic peptide
  MOD_RES (1)..(1): N-terminal ACETYLATION
  MOD_RES (4)..(4): 1-methyl-L-tryptophan
  MOD_RES (7)..(7): 5-fluoro-L-tryptophan
  MOD_RES (13)..(13): C-terminal CONH2
ICVWQDWGAHRCT

SEQ ID NO: 33 - protein, 15 aa, Artificial Sequence
  Description of Artificial Sequence: Synthetic peptide
  MOD_RES (1)..(1): N-term H
  MOD_RES (5)..(5): 6-fluoro-L-tryptophan
  MOD_RES (8)..(8): 6-fluoro-L-tryptophan
  MOD_RES (15)..(15): C-terminal COOH
GICVWQDWGAHRCTN

SEQ ID NO: 34 - protein, 13 aa, Artificial Sequence
  Description of Artificial Sequence: Synthetic peptide
  MOD_RES (1)..(1): N-terminal ACETYLATION
  MOD_RES (4)..(4): 1-formyl-Trp
  MOD_RES (13)..(13): C-terminal CONH2
ICVWQDWGAHRCT

SEQ ID NO: 35 - protein, 13 aa, Artificial Sequence
  Description of Artificial Sequence: Synthetic peptide
  MOD_RES (1)..(1): N-terminal acetylation
  MOD_RES (4)..(4): 1-methoxy-tryptophan
  MISC_FEATURE (13)..(13): C-terminal CONH2
ICVWQDWGAHRCT

SEQ ID NO: 36 - protein, 15 aa, Artificial Sequence
  Description of Artificial Sequence: Synthetic peptide
  MOD_RES (1)..(1): N-term H
  MOD_RES (5)..(5): 5-fluoro-L-tryptophan
  MOD_RES (8)..(8): 5-fluoro-L-tryptophan
  MOD_RES (15)..(15): C-terminal COOH
GICVWQDWGAHRCTN

SEQ ID NO: 37 - protein, 15 aa, Artificial Sequence
  Description of Artificial Sequence: Synthetic peptide
  MISC_FEATURE: see specification as filed for detailed description of substitutions and preferred embodiments
  VARIANT (1)..(1): Any amino acid
  MOD_RES (1)..(1): BLOCKED
  VARIANT (2)..(4): Any independently selected amino acid or amino acid analog
  VARIANT (5)..(5): Trp or analog of Trp
  VARIANT (8)..(8): Trp or analog of Trp
  VARIANT (10)..(14): Any independently selected amino acid or amino acid analog
  VARIANT (15)..(15): Any amino acid
  MOD_RES (15)..(15): BLOCKED
XXXXXQDXGXXXXXX

SEQ ID NO: 38 - protein, 18 aa, Artificial Sequence
  Description of Artificial Sequence: Synthetic peptide
  MISC_FEATURE: see specification as filed for detailed description of substitutions and preferred embodiments
  VARIANT (1)..(1): Any amino acid
  MOD_RES (1)..(1): BLOCKED
  VARIANT (2)..(3): Ile, Val, Leu, B1-Ile, B1-Val, B1-Leu or a dipeptide comprising Gly-Ile or B1-Gly-Ile, wherein B1 represents a first blocking moiety
  misc_feature (2)..(3): This region may encompass 1 to 2 residues, wherein some positions may be absent
  DISULFID (4)..(14)
  VARIANT (6)..(6): Independently selected from Trp or analogs of Trp
  VARIANT (9)..(9): Independently selected from Trp or analogs of Trp
  VARIANT (11)..(11): His, Ala or an analog of Ala, Phe, Trp or an analog of Trp
  VARIANT (15)..(17): L-Thr, D-Thr, Ile, Val, Gly, a dipeptide selected from Thr-Ala and Thr-Asn, or a tripeptide comprising Thr-Ala-Asn, wherein a C-terminal -OH of any of the L-Thr, D-Thr, Ile, Val, Gly, Ala or Asn is optionally replaced by a second blocking moiety
  misc_feature (15)..(17): This region may encompass 1 to 3 residues, wherein some positions may be absent
  VARIANT (18)..(18): Any amino acid
  MOD_RES (18)..(18): BLOCKED
XXXCVXQDXGXHRCXXXX

SEQ ID NO: 39 - protein, 15 aa, Artificial Sequence
  Description of Artificial Sequence: Synthetic peptide
  MISC_FEATURE: see specification as filed for detailed description of substitutions and preferred embodiments
  VARIANT (1)..(1): Any amino acid
  MOD_RES (1)..(1): BLOCKED
  VARIANT (2)..(2): Ile or Gly
  VARIANT (3)..(3): Ile or Cys
  VARIANT (4)..(4): Val or Cys
  VARIANT (5)..(5): Val, Tyr, Trp, 2-Nal, 1-Nal, 2-Igl, Dht, Bpa, Bta, 5f-Trp, 5-methyl-Trp, 1-methyl-Trp, 1-formyl-Trp or 1-methyoxy-Trp
  VARIANT (6)..(6): Gln, Trp, 6f-Trp or 5f-Trp
  VARIANT (7)..(7): Asp or Gln
  VARIANT (8)..(8): Trp, Asp or 5f-Trp
  VARIANT (9)..(9): Gly, Trp, 6f-Trp or 5f-Trp
  VARIANT (10)..(10): His, Ala, 2-Abu or Gly
  VARIANT (11)..(11): His or Ala
  VARIANT (12)..(12): Arg or His
  VARIANT (13)..(13): Cys or Arg
  VARIANT (14)..(14): Thr, d-Thr or Cys
  VARIANT (15)..(15): Any amino acid
  MOD_RES (15)..(15): BLOCKED
XXXXXXXXXXXXXXX

SEQ ID NO: 40 - protein, 15 aa, Artificial Sequence
  Description of Artificial Sequence: Synthetic peptide
  MISC_FEATURE: see specification as filed for detailed description of substitutions and preferred embodiments
  VARIANT (1)..(1): Any amino acid
  MOD_RES (1)..(1): BLOCKED
  MOD_RES (1)..(1): BLOCKED
  DISULFID (3)..(13)
  MOD_RES (5)..(5): Trp(Me)
  MOD_RES (9)..(9): N-methyl glycine (sarcosine)
  MOD_RES (14)..(14): N-methyl isoleucine
  VARIANT (15)..(15): Any amino acid
  MOD_RES (15)..(15): BLOCKED
XICVWQDWGAHRCIX

SEQ ID NO: 41 - protein, 15 aa, Artificial Sequence
  Description of Artificial Sequence: Synthetic peptide
  MISC_FEATURE: see specification as filed for detailed description of substitutions and preferred embodiments
  VARIANT (1)..(1): Any amino acid
  MOD_RES (1)..(1): BLOCKED
  DISULFID (3)..(13)
  MOD_RES (5)..(5): Trp(Me)
  MOD_RES (9)..(9): N-methyl glycine (sarcosine)
  MOD_RES (14)..(14): N-methyl isoleucine
  VARIANT (15)..(15): Any amino acid
  MOD_RES (15)..(15): BLOCKED
XICVWQDWGAHRCIX

SEQ ID NO: 42-58 - skipped (17 entries)

SEQ ID NO: 59 - protein, 5 aa, Artificial Sequence
  Description of Artificial Sequence: Synthetic peptide
  misc_feature (1)..(5): Cyclic
  MOD_RES (1)..(1): Ornithine
  MOD_RES (3)..(3): D-cyclohexylalanine
XPAWR

SEQ ID NO: 60 - protein, 5 aa, Artificial Sequence
  Description of Artificial Sequence: Synthetic peptide
  misc_feature (1)..(5): Cyclic
  MOD_RES (3)..(3): D-cyclohexylalanine
KPAWR

SEQ ID NO: 61 - protein, 10 aa, Artificial Sequence
  Description of Artificial Sequence: Synthetic peptide
  VARIANT (1)..(5): Any amino acid. This region may encompass 1 to 5 residues, wherein some positions may be absent.
  MOD_RES (6)..(6): Ornithine
  misc_feature (6)..(10): Cyclic
  MOD_RES (8)..(8): D-cyclohexylalanine
XXXXXXPAWR

SEQ ID NO: 62 - protein, 10 aa, Artificial Sequence
  Description of Artificial Sequence: Synthetic peptide
  VARIANT (1)..(5): Any amino acid. This region may encompass 1 to 5 residues, wherein some positions may be absent
  misc_feature (6)..(10): Cyclic
  MOD_RES (8)..(8): D-cyclohexylalanine
XXXXXKPAWR

SEQ ID NO: 63 - protein, 6 aa, Artificial Sequence
  Description of Artificial Sequence: Synthetic peptide
  MOD_RES (2)..(2): Ornithine
  misc_feature (2)..(6): Cyclic
  MOD_RES (4)..(4): D-cyclohexylalanine
FXPAWR

SEQ ID NO: 64 - protein, 6 aa, Artificial Sequence
  Description of Artificial Sequence: Synthetic peptide
  misc_feature (2)..(6): Cyclic
  MOD_RES (4)..(4): D-cyclohexylalanine
FKPAWR

SEQ ID NO: 65 - protein, 5 aa, Artificial Sequence
  Description of Artificial Sequence: Synthetic peptide
  MOD_RES (1)..(1): cinnamoylated
  MOD_RES (1)..(1): Ornithine
  misc_feature (1)..(5): Cyclic
  MOD_RES (3)..(3): D-cyclohexylalanine
XPAWR

SEQ ID NO: 66 - protein, 5 aa, Artificial Sequence
  Description of Artificial Sequence: Synthetic peptide
  MOD_RES (1)..(1): H-cinnamoylated
  MOD_RES (1)..(1): Ornithine
  misc_feature (1)..(5): Cyclic
  MOD_RES (3)..(3): D-cyclohexylalanine
XPAWR

SEQ ID NO: 67 - protein, 6 aa, Artificial Sequence
  Description of Artificial Sequence: Synthetic peptide
  MOD_RES (1)..(1): N-terminal ACETYLATION
  MOD_RES (2)..(2): Ornithine
  misc_feature (2)..(6): Cyclic
  MOD_RES (4)..(4): D-cyclohexylalanine
FXPAWR

SEQ ID NO: 68 - protein, 10 aa, Artificial Sequence
  Description of Artificial Sequence: Synthetic peptide
WESNGQPENN

SEQ ID NO: 69 - protein, 17 aa, Artificial Sequence
  Description of Artificial Sequence: Synthetic peptide
KTISKAKGQPREPQVYT

(19) United States
(12) Patent Application Publication
(10) Pub. No.: US 2020/0155675 A1
(43) Pub. Date: May 21, 2020

(54) METHODS AND COMPOSITIONS FOR CANCER TREATMENT AND TREATMENT SELECTION

(71) Applicant: Apellis Pharmaceuticals, Inc., Crestwood, KY (US)

(72) Inventor: Cedric Francois, Prospect, KY (US)

(21) Appl. No.: 16/425,269

(22) Filed: May 29, 2019

Related U.S. Application Data
(62) Division of application No. 15/513,397, filed on Mar. 22, 2017, now abandoned.
(60) Provisional application No. 62/054,939, filed on Sep. 24, 2014.

(51) Int. Cl. A61K 39/395; G01N 33/574; C12Q 1/6886; C07K 16/28; A61K 47/10; A61K 45/06; A61K 38/17

(52) U.S. Cl. CPC A61K 39/39558; G01N 33/57492; C12Q 1/6886; C07K 16/2827; C07K 16/2818; A61K 47/10; C07K 2317/76; A61K 38/1709; C12Q 2600/158; C12Q 2600/156; C12Q 2600/118; C12Q 2600/106; A61K 45/06

(57) ABSTRACT

In some aspects, the disclosure provides methods of treating cancer or an infection. In some aspects, the disclosure provides methods of enhancing the efficacy of treatment with an immune checkpoint inhibitor. In some embodiments the methods comprise administering a complement inhibitor and an immune checkpoint inhibitor to a subject with cancer or an infection. In some aspects, the disclosure provides methods of identifying a subject who is an appropriate candidate for treatment with an immune checkpoint inhibitor. In some aspects, the disclosure provides methods of identifying a subject who is an appropriate candidate for treatment with an immune checkpoint inhibitor and a complement inhibitor. In some embodiments the methods comprise determining whether the subject is an appropriate candidate for treatment with an immune checkpoint inhibitor and a complement inhibitor based on an assay of a complement system biomarker in the subject or in a sample obtained from the subject.

### CROSS REFERENCE TO RELATED APPLICATIONS

The present application claims priority to U.S. Provisional Patent Application No. 62/054,939, filed Sep. 24, 2014, the entire contents of which are hereby incorporated by reference.

### BACKGROUND

Cancer is among the leading causes of death worldwide, accounting for an estimated 8.2 million deaths in 2012 (Globocan 2012, International Agency for Research on Cancer (IARC), World Health Organization, IARC website). The mainstays of treatment for many types of cancer remain conventional chemotherapeutic agents and radiotherapy, which act on dividing cells in a relatively non-specific manner. Most therapeutic agents that have been approved in recent years for treatment of cancer confer only modest increases in average life expectancy and/or have relatively narrow indication(s). There is significant unmet need for innovative approaches for treating cancer. There is a particular need for improved treatment for patients with advanced cancer.

### SUMMARY

Immune checkpoint inhibitors are a class of agents that have recently been shown to produce unprecedented rates of prolonged disease-free response in certain cancers, including some that are refractory to other therapeutic approaches. However, not all subjects with cancer benefit from these agents. In some aspects, the present invention embodies the insight that, in at least some cases, the response of cancer patients to checkpoint inhibitor therapy may be impacted by complement. Without wishing to be bound by any particular theory, the present disclosure proposes, for example, that complement may play a role in resistance to treatment with immune checkpoint inhibitors, which occurs in a significant percentage of subjects treated with such agents. Moreover, the present disclosure provides the insight that a contributing factor to a subject's ability to respond to checkpoint inhibitor therapy and/or the nature or extent of response to checkpoint inhibitor therapy may be the complement status of the subject. Assessment of the complement status of a subject may be performed using a variety of assays, and the results may be used in the treatment of cancer patients. For example, based at least in part on complement status, a patient may be treated with a checkpoint inhibitor as a single agent or may be treated with a combination therapy such as an immune checkpoint inhibitor and a complement inhibitor.

In some aspects, described herein are methods of identifying a subject who is an appropriate candidate for treatment with an immune checkpoint inhibitor. In some embodiments the methods comprise determining whether the subject is an appropriate candidate for treatment with an immune checkpoint inhibitor based on an assay of a complement system biomarker in the subject or in a sample obtained from the subject. In some embodiments the assay comprises determining the genotype of the subject with respect to one or more polymorphisms of a complement-related gene, wherein an allele of the polymorphism is associated with an increased likelihood of responsiveness (or a decreased likelihood of nonresponsiveness) to treatment with an immune checkpoint inhibitor and identifying the subject as an appropriate candidate for treatment with an immune checkpoint inhibitor if the subject harbors the allele. In some embodiments the assay comprises determining the genotype of the subject with respect to one or more polymorphisms of a complement-related gene, wherein an allele of the polymorphism is associated with a decreased likelihood of responsiveness (or an increased likelihood of nonresponsiveness) to treatment with an immune checkpoint inhibitor and identifying the subject as an appropriate candidate for treatment with an immune checkpoint inhibitor if the subject does not harbor the allele. In some embodiments the assay comprises determining the genotype of the subject with respect to one or more polymorphisms of a complement-related gene, wherein an allele of the polymorphism is associated with a decreased likelihood of responsiveness (or an increased likelihood of nonresponsiveness) to treatment with an immune checkpoint inhibitor and identifying the subject as not being an appropriate candidate for treatment with an immune checkpoint inhibitor if the subject does not harbor the allele. In some embodiments the polymorphism is one that is associated with risk of a complement-mediated disorder.

In some aspects, the disclosure provides methods of identifying subjects who are appropriate candidates for treatment with an immune checkpoint inhibitor and a complement inhibitor. In some embodiments the methods comprise determining whether the subject is an appropriate candidate for treatment with an immune checkpoint inhibitor and a complement inhibitor based on an assay of a complement system biomarker in the subject or in a sample obtained from the subject. In some embodiments the assay comprises determining the genotype of the subject with respect to one or more polymorphisms of a complement-related gene, wherein an allele of the polymorphism is associated with a decreased likelihood of responsiveness (or an increased likelihood of nonresponsiveness) to treatment with an immune checkpoint inhibitor and identifying the subject as an appropriate candidate for treatment with an immune checkpoint inhibitor and a compement inhibitor if the subject harbors the allele. In some embodiments the polymorphism is one that is associated with risk of a complement-mediated disorder.

In some aspects, the disclosure provides methods of identifying subjects who are appropriate candidates for treatment with an immune checkpoint inhibitor and a complement inhibitor. In some embodiments the methods comprise determining whether the subject is an appropriate candidate for treatment with an immune checkpoint inhibitor and a complement inhibitor based on an assay of a complement system biomarker in the subject or in a sample obtained from the subject. In some embodiments the assay comprises determining the genotype of the subject with respect to one or more polymorphisms of a complement-related gene, wherein an allele of the polymorphism is associated with a decreased likelihood of responsiveness (or an increased likelihood of nonresponsiveness) to treatment with an immune checkpoint inhibitor and identifying the subject as an appropriate candidate for treatment with an immune checkpoint inhibitor and a compement inhibitor if the subject harbors the allele. In some embodiments the polymorphism is one that is associated with risk of a complement-mediated disorder.

In some aspects, described herein is a method of treating a subject in need of treatment of cancer, the method comprising treating the subject with a combination of an immune checkpoint inhibitor and a complement inhibitor to the subject (i.e., administering therapy with a complement inhibitor, an immune checkpoint inhibitor, or both, so that the subject receives therapy with both). In some embodiments (i) the subject has been determined to have a complement system biomarker profile that correlates with a lack of response from monotherapy with an immune checkpoint inhibitor; (ii) the subject has been determined, based on an assay of one or more complement system biomarkers, to be unlikely to respond to therapy with an immune checkpoint inhibitor; (iii) the subject has been previously treated with an immune checkpoint inhibitor and did not respond; (iv) the subject has been previously treated for the cancer with an immune checkpoint inhibitor in the absence of combination treatment with a complement inhibitor and did not respond; (v) the subject has previously treated for the cancer with an immune checkpoint inhibitor in the absence of combination treatment with a complement inhibitor and exhibited a response followed by disease progression; or (vi) any combination of (i)-(v).

In some embodiments of combination therapy with an immune checkpoint inhibitor and a complement inhibitor, the immune checkpoint inhibitor and the complement inhibitor are administered at least once within 6 weeks of each other. In some embodiments the immune checkpoint inhibitor and the complement inhibitor are administered at least once within 6 months of each other.

In some embodiments of combination therapy with an immune checkpoint inhibitor and a complement inhibitor, the subject is further treated with a second anti-cancer agent. In some embodiments the second anti-cancer agent comprises conventional chemotherapy, a molecularly targeted anticancer agent, a cancer vaccine, a second immunostimulatory agent, cell-based immunotherapy, or a combination thereof to the subject.

In some embodiments of combination therapy with an immune checkpoint inhibitor and a complement inhibitor, the method further comprises determining, based on an assay of a complement system biomarker in the subject or in a sample obtained from the subject, that the subject is not an appropriate candidate for standard treatment with an immune checkpoint inhibitor or that the subject is an appropriate candidate for therapy with an immune checkpoint inhibitor and a complement inhibitor.

In some aspects, described herein is a method of treating a subject in need of treatment for cancer comprising: (a) determining based on an assay of a complement system biomarker in the subject or in a sample obtained from the subject, that the subject is an appropriate candidate for standard treatment with an immune checkpoint inhibitor; and (b) treating the subject with an immune checkpoint inhibitor. In some embodiments step (a) comprises determining that the subject is an appropriate candidate for standard treatment with an immune checkpoint inhibitor. In some embodiments step (a) comprises determining that the subject is an appropriate candidate for standard treatment with an immune checkpoint inhibitor, and step (b) comprises treating the subject with an immune checkpoint inhibitor and not with a complement inhibitor. In some embodiments step (a) comprises determining that the subject is an appropriate candidate for standard treatment with an immune checkpoint inhibitor, and step (b) comprises treating the subject with an immune checkpoint inhibitor and with a complement inhibitor. In certain such embodiments, although the subject is determined to be an appropriate candidate for standard treatment with an immune checkpoint inhibitor, the subject may benefit from combination therapy with an immune checkpoint inhibitor and a complement inhibitor.

In some aspects, described herein is a method of treating a subject in need of treatment for cancer comprising: (a) determining based on an assay of a complement system biomarker in the subject or in a sample obtained from the subject, that the subject is not an appropriate candidate for standard therapy with an immune checkpoint inhibitor; and (b) (i) treating the subject with an immune checkpoint inhibitor and a complement inhibitor or (ii) treating the subject with a second anti-cancer therapy in combination with or instead of an immune checkpoint inhibitor.

In some aspects, described herein is a method of treating a cancer patient comprising steps of: (a) obtaining results of an assay of one or more a complement system biomarker in a cancer patient or in a sample obtained from a cancer patient; (b) correlating the results with the likelihood that the patient will respond to therapy with an immune checkpoint inhibitor; and (c) based on step (b) either (i) treating the cancer patient with an immune checkpoint inhibitor or (ii) treating the cancer patient with an immune checkpoint inhibitor and a complement inhibitor or (iii) treating the cancer patient with a second anti-cancer therapy in combination with or instead of an immune checkpoint inhibitor.

In some embodiments of any of the methods, the assay of a complement system biomarker comprises determining the genotype of the subject or cancer with respect to a polymorphism in or near of a complement-related gene.

In some embodiments of any of the methods or compositions that involve or relate to a complement system biomarker or an assay of a complement system biomarker, results of the assay of a complement system biomarker comprise the genotype of the subject or cancer with respect to a polymorphism in or near of a complement-related gene. In some embodiments the assay of a complement system biomarker comprises determining the genotype of the subject or cancer with respect to a mutation associated with a complement-mediated disorder. In some embodiments results of the assay of a complement system biomarker comprise the genotype of the subject or cancer with respect to a mutation associated with a complement-mediated disorder.

In some embodiments of any of the methods or compositions that involve or relate to a complement system biomarker or an assay of a complement system biomarker, the complement system biomarker is a complement-related gene. In some embodiments, the complement system biomarker is a complement-related protein.

In some embodiments of any of the methods, the complement system biomarker serves as an indicator of the level of complement activation in the subject.

In some embodiments of any of the methods, the complement system biomarker is measured in a cancer or in a sample obtained from a cancer.

In some embodiments of any of the methods, the complement system biomarker serves as an indicator of the level of complement activation in the cancer.

In some embodiments of any of the methods, the method comprises providing a biological sample obtained from the subject; and performing an assay to detect, measure, or characterize at least one complement system biomarker in the biological sample.

In some embodiments of any of the methods, the method comprises obtaining a biological sample from the cancer; and performing an assay to detect, measure, or characterize at least one complement system biomarker in the biological sample.

In some aspects, described herein is a method of method of treating a cancer patient comprising: obtaining the genotype of the cancer patient with respect to at least one polymorphism in or within up to 150 kilobases (kB) of a complement-related gene; determining that the patient is an appropriate candidate for treatment with an immune checkpoint inhibitor based on the genotype; and treating the patient with an immune checkpoint inhibitor.

In some aspects, described herein is a method of method of treating a cancer patient comprising: obtaining the genotype of the patient with respect to at least one polymorphism in or near a complement-related gene; determining that the patient is not an appropriate candidate for standard treatment with immune checkpoint inhibitor based on the genotype; and (i) treating the patient with an immune checkpoint inhibitor and a complement inhibitor; or (ii) treating the patient with a second anti-cancer therapy in combination with or instead of an immune checkpoint inhibitor.

In some aspects, described herein is a method of treating a cancer patient comprising: (a) obtaining the genotype of a cancer patient with respect to at least one polymorphism or mutation in or near a complement-related gene; (b) correlating the genotype with the likelihood that the cancer patient will respond to therapy with an immune checkpoint inhibitor; and (c) based on step (b) either (i) treating the cancer patient with an immune checkpoint inhibitor (ii) treating the cancer patient with an immune checkpoint inhibitor and a complement inhibitor or (iii) treating the cancer patient with a second anti-cancer therapy other than a complement inhibitor, in combination with or instead of an immune checkpoint inhibitor based on step (b).

In some embodiments of any of the methods, one or more of the polymorphisms or mutations is associated with susceptibility to a complement-mediated disorder. In some embodiments the complement-mediated disorder is age-related macular degeneration (AMD).

In some embodiments of any of the methods, one or more of the polymorphisms or mutations is in a complement-related gene. In some embodiments of any of the methods one or more of the polymorphisms or mutations is in a complement-related gene that encodes a complement protein. In some embodiments of any of the methods, one or more of the polymorphisms or mutations is in a complement-related gene that encodes a complement regulatory protein. In some embodiments of any of the methods, one or more of the polymorphisms or mutations is in or near a complement-related gene that encodes complement factor H (CFH), complement factor I (CFI), complement component C3 (C3), complement component C2 (C2), complement factor B (CFB), complement component C7 (C7), and mannose binding lectin 2 (MBL-2). In some embodiments of any of the methods, one or more of the polymorphisms is rs10737680 (CFH), rs429608 (C2/CFB), rs2230199 (C3), rs4698775 (CFI), or a polymorphism in linkage disequilibrium with any of these. Certain embodiments of any of the methods may involve or relate to any one or more of the polymorphisms or mutations, or any combination thereof.

In some aspects, described herein is a method of treating a subject in need of treatment for cancer comprising: (a) treating the subject with an immune checkpoint inhibitor; (b) evaluating the subject one or more times after initiating treatment with the immune checkpoint inhibitor; (c) determining that the subject exhibits progressive disease; and (d) treating the subject with a complement inhibitor in combination with the same or a different immune checkpoint inhibitor. In some embodiments the method comprises treating the subject with a complement inhibitor if the subject does not respond to the immune checkpoint inhibitor within 6 months of initiating treatment or exhibits progressive disease after an initial response.

In some embodiments of any of the methods, the cancer is a melanoma, lung cancer, bladder cancer, head and neck cancer, ovarian cancer, renal cell carcinoma (RCC), prostate cancer, or hematological malignancy.

In some embodiments of any of the methods, the cancer is a metastatic cancer, optionally an unresectable cancer, stage III cancer, or stage IV cancer.

In some aspects, described herein is a pharmaceutical composition comprising a complement inhibitor and an immune checkpoint inhibitor.

In some embodiments of any of the methods or compositions, the immune checkpoint inhibitor comprises an antibody (e.g., a monoclonal antibody), an engineered binding protein, a soluble receptor, an aptamer, a peptide, or a small molecule that binds to an immune checkpoint protein.

In some embodiments of any of the methods or compositions, the immune checkpoint inhibitor comprises an antagonist of an inhibitory receptor.

In some embodiments of any of the methods or compositions, the immune checkpoint inhibitor inhibits the PD1 pathway.

In some embodiments of any of the methods or compositions, the immune checkpoint inhibitor binds to PD1, PD-L1 or PD-L2.

In some embodiments of any of the methods or compositions, the immune checkpoint inhibitor comprises an antibody that binds to PD1 or PD-L1.

In some embodiments of any of the methods or compositions, the immune checkpoint inhibitor comprises nivolumab, pembrolizumab (lambrolizumab), pidilizumab, MEDI0680, MPDL3280A, AMP-224, BMS-936559, MPDL3280A, MEDI4736, MSB0010718C, or any combination of these.

In some embodiments of any of the methods or compositions, the checkpoint inhibitor inhibits the CTLA4 pathway. For example, in some embodiments the immune checkpoint inhibitor binds to CTLA4. In some embodiments the immune checkpoint inhibitor comprises ipilimumab or tremelimumab.

In some embodiments of any of the methods or compositions, the immune checkpoint inhibitor inhibits a killer-like immunoglobulin receptor (KIR) pathway. For example, in some embodiments the immune checkpoint inhibitor binds to a KIR or KIR ligand.

In some embodiments of any of the methods or compositions, the immune checkpoint inhibitor inhibits an immune checkpoint pathway involving LAG3, TIM3, BTLA, A2AR, or A2BR.

In some embodiments of any of the methods or compositions the complement inhibitor comprises an antibody (e.g., a monoclonal antibody), aptamer, an engineered binding protein, peptide, polypeptide, or small molecule that binds to a complement component. In some embodiments the complement inhibitor binds to C3, C5, factor B, or factor D. In some embodiments the complement inhibitor inhibits cleavage of C3, C5, or factor B. In some embodiments the complement inhibitor comprises eculizumab, pexelizumab, lampalizumab, or a compstatin analog. In some embodiments the complement inhibitor comprises a compstatin analog. In some embodiments the complement inhibitor comprises a compstatin analog whose sequence comprises any of SEQ ID NOs: 3-41. In some embodiments the complement inhibitor comprises a compstatin analog whose sequence comprises SEQ ID NO: 14, 21, 28, 29, 32, 33, 34, or 36. In some embodiments the complement inhibitor comprises a clearance-reducing moiety, optionally wherein the clearance reducing moiety comprises PEG.

In some embodiments of any of the methods or compositions, the immune checkpoint inhibitor comprises ipilimumab, tremilimumab, nivolumab, pembrolizumab (lambrolizumab), pidilizumab, MPDL3280A (an Fc engineered anti-PD-L1), BMS-936559, MPDL3280A, MEDI4736, MSB0010718C, or any combination of these, optionally wherein the combination comprises no more than 2, 3, or 4 immune checkpoint inhibitors.

In some embodiments of any aspect, a response in a subject being treated for cancer is defined according to the immune-related response criteria.

All articles, books, patent applications, patents, other publications, websites, and databases mentioned in this application are incorporated herein by reference. In the event of a conflict between the specification and any of the incorporated references the specification (including any amendments thereto) shall control. Unless otherwise indicated, art-accepted meanings of terms and abbreviations are used herein.

### DETAILED DESCRIPTION OF CERTAIN EMBODIMENTS

### I. Glossary

An “allele” is any of two or more alternative forms of a gene or more generally, any of two or more alternative forms of a portion of genomic DNA, which portion may be as little as a single nucleotide in length and may or may not be located within a gene. For example, the alternate forms of a polymorphism such as a single nucleotide polymorphism (SNP) may be referred to in the art as “alleles” or “alleles of the polymorphism”. A “major allele” is the allele that occurs most frequently in the general population. As used herein, a “minor allele” is an allele that occurs less frequently than the major allele. If there are three or more alleles, the second most frequently occurring allele is considered to be the minor allele.

As used herein, the term “antibody” refers to an immunoglobulin and encompasses full size antibodies and antibody fragments comprising an antigen binding site. Antibodies useful in certain embodiments of the invention may originate from or be derived from a mammal, e.g., a human, non-human primate, rodent (e.g., mouse, rat, rabbit), goat, camelid, or from a bird (e.g., chicken), and may be of any of the various antibody isotypes, e.g., the mammalian isotypes: IgG (e.g., of the IgG1, IgG2, IgG3, or IgG4 subclass), IgM, IgA, IgD, and IgE or isotypes that are not found in mammals, e.g., IgY (found in birds) or IgW (found in sharks). An antibody fragment (Fab) may be, for example, a Fab′, F(ab′)₂, scFv (single-chain variable), single domain antibody (e.g., a VHH), or other fragment that retains or contains an antigen binding site. See, e.g., Allen, T., Nature Reviews Cancer, Vol. 2, 750-765, 2002, and references therein. Antibodies known in the art as diabodies, minibodies, or nanobodies can be used in various embodiments. Bispecific or multispecific antibodies may be used in various embodiments. The heavy and light chain of IgG immunoglobulins (e.g., rodent or human IgGs) contain four framework regions (FR1 through FR4) separated respectively by three complementarity determining regions (CDR1 through CDR3). The CDRs, particularly the CDR3 regions, especially the heavy chain CDR3, are largely responsible for antibody specificity. An antibody may be a chimeric antibody in which, for example, a variable domain of non-human origin, e.g., of rodent (e.g., murine) or non-human primate origin) is fused to a constant domain of human origin, or a “humanized” antibody in which some or all of the complementarity-determining region (CDR) amino acids that constitute an antigen binding site (sometimes along with one or more framework amino acids or regions) are “grafted” from a rodent antibody (e.g., murine antibody) or phage display antibody to a human antibody, thus retaining the specificity of the rodent or phage display antibody. Thus, humanized antibodies may be recombinant proteins in which only the antibody complementarity-determining regions are of non-human origin. It will be appreciated that the alterations to antibody sequence that are involved in the humanization process are generally carried out through techniques at the nucleic acid level, e.g., standard recombinant nucleic acid techniques. In some embodiments only the specificity determining residues (SDRs), the CDR residues that are most crucial in the antibody-ligand interaction, are grafted. The SDRs may be identified, e.g., through use of a database of the three-dimensional structures of the antigen-antibody complexes of known structures or by mutational analysis of the antibody-combining site. In some embodiments an approach is used that involves retention of more CDR residues, namely grafting of so-called “abbreviated” CDRs, the stretches of CDR residues that include all the SDRs. See, e.g., Kashmiri, S V, Methods. 36(1):25-34 (2005), for further discussion of SDR grafting. See, e.g., Almagro J C, Fransson J. Humanization of antibodies. Front Biosci. 13:1619-33 (2008) for review of various methods of obtaining humanized antibodies. It will be understood that “originate from or derived from” refers to the original source of the genetic information specifying an antibody sequence or a portion thereof, which may be different from the species in which an antibody is initially synthesized. For example, “human” domains may be generated in rodents (e.g., mice) whose genome incorporates human immunoglobulin genes or may be generated using phage display. See, e.g., Vaughan, et al, (1998), Nature Biotechnology, 16: 535-539, e.g., for discussion of methods that may be used to generate a fully human antibody. It will be understood that the amino acid sequences of the variable regions of such antibodies are sequences that, while derived from and related to the germline sequences encoding variable domains (V_{H} and/or V_{L} domains) of a particular species (e.g., human), may not naturally exist within that species' antibody germline repertoire in vivo. For example, the human immunoglobulin genes may have been subjected to in vitro mutagenesis (or, when an animal transgenic for human immunoglobulin gene sequences is used, in vivo somatic mutagenesis). An antibody may be polyclonal or monoclonal, though for purposes of the present invention monoclonal antibodies are generally preferred as therapeutic agents. Antibodies may be glycosylated or non-glycosylated. Methods for generating antibodies that specifically bind to virtually any molecule of interest are known in the art. For example, monoclonal or polyclonal antibodies can be purified from natural sources, e.g., from blood or ascites fluid of an animal that produces the antibody (e.g., following immunization with the molecule or an antigenic fragment thereof) or can be produced recombinantly, in cell culture and, e.g., purified from culture medium. Affinity purification may be used, e.g., protein A/G affinity purification and/or affinity purification using the antigen as an affinity reagent. Suitable antibodies can be identified using phage display and related techniques. See, e.g., Kaser, M. and Howard, G., “Making and Using Antibodies: A Practical Handbook” and Sidhu, S., “Phage Display in Biotechnology and Drug Discovery”, CRC Press, Taylor and Francis Group, 2005, for further information. Methods for generating antibody fragments are well known. For example, F(ab′)₂ fragments can be generated, for example, through the use of an Immunopure F(ab′)₂ Preparation Kit (Pierce) in which the antibodies are digested using immobilized pepsin and purified over an immobilized Protein A column. The digestion conditions (such as temperature and duration) may be optimized by one of ordinary skill in the art to obtain a good yield of F(ab′)₂. The yield of F(ab′)₂ resulting from the digestion can be monitored by standard protein gel electrophoresis. F(ab′) can be obtained by papain digestion of antibodies, or by reducing the S—S bond in the F(ab′)₂. As used herein, a “single-chain Fv” or “scFv” antibody fragment comprises the V_{H} and V_{L} domains of an antibody, wherein these domains are present in a single polypeptide chain. Typically, a scFv antibody further comprises a polypeptide linker between the V_{H} and V_{L} domains, although other linkers could be used to connect the domains in certain embodiments.

The terms “approximately” or “about” in reference to a number generally include numbers that fall within ±10%, in some embodiments ±5%, in some embodiments ±1%, in some embodiments ±0.5% of the number unless otherwise stated or otherwise evident from the context (except where such number would impermissibly exceed 100% of a possible value).

The term “cancer”, also known as a malignant tumor, refers to any of a group of diseases involving abnormal cell proliferation with the potential to invade locally and/or spread to other parts of the body (metastasize). The term “cancer” is generally used interchangeably with “tumor” herein (unless a tumor is specifically referred to as a “benign” tumor, which is an abnormal mass of cells that lacks the ability to invade neighboring tissue or metastasize), and encompasses malignant solid tumors (e.g., carcinomas, sarcomas) and malignant growths in which there may be no detectable solid tumor mass (e.g., certain hematologic malignancies).

“Complement activation capacity” refers to the level of complement activation that would result from exposure to a stimulus that causes maximum complement activation. Typically, complement activation capacity is assessed using a sample obtained from a subject (e.g., a blood, plasma, serum, or other fluid sample, which may be diluted appropriately), which sample may be exposed in vitro to a complement activating stimulus. A heat-inactivated sample can be used as a control. It will be understood that the stimulus need not be sufficient to cause maximum complement activation in order to provide a measurement of complement activation capacity. For example, the extent to which complement activation occurs within a defined time period can provide an indication of complement activation capacity.

A “complement component” or “complement protein” is a protein that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, and properdin. Components of the lectin pathway include, e.g., MBL2, MASP-1, and MASP-2. Complement components also include cell-bound receptors for soluble complement components, wherein such receptor mediates one or more biological activities of such soluble complement component following binding of the soluble complement component. Such receptors include, e.g., C5a receptor (C5aR), C5a receptor 2 (C5aR2, often referred to as C5L2) C3a receptor (C3aR), Complement Receptor 1 (CR1), Complement Receptor 2 (CR2), Complement Receptor 3 (CR3, also known as CD45), etc. It will be appreciated that the term “complement component” is not intended to include those molecules and molecular structures that serve as “triggers” for complement activation, e.g., antigen-antibody complexes, foreign structures found on microbial or artificial surfaces, etc.

A “complement regulatory protein” is a protein involved in regulating complement activity. A complement regulatory protein may down-regulate complement activity by, e.g., inhibiting complement activation or by inactivating or accelerating decay of one or more activated complement proteins. Examples of complement regulatory proteins include C1 inhibitor, C4 binding protein, clusterin, vitronectin, complement factor H (CFH, sometimes referred to as FH), complement factor I (CFI, sometimes referred to as FI or IF), and the cell-bound proteins CD46, CD55, CD59, CR1, CR2, and CR3. In certain embodiments C-reactive protein is considered a complement regulatory protein. In certain embodiments C-reactive protein is not considered a complement regulatory protein.

A “complement control protein” is a complement regulatory protein comprising multiple SCR modules as described below. Examples include CFH, CD46, CD55, CR1, and CR2.

A “complement-like protein” is a protein that has significant sequence identity to a complement protein or a complement control protein over at least 20% of its length and/or specifically competes with the complement protein or complement control protein for binding to its target, e.g., has an affinity at least 10% as great. The genes encoding such proteins may be found in close proximity to genes encoding the complement protein or complement control protein having a similar sequence. For example, the CFH gene cluster contains numerous CFH-like genes (e.g., CFHR1, CFHR2, CFHR3, CFHR4, and CFHR5).

“Complement-related protein” refers collectively to complement components, complement regulatory proteins, and complement-like proteins; however, wherever the disclosure refers to complement-related proteins in general, it is understood that the invention encompasses embodiments that relate specifically to complement components, complement regulatory proteins, complement-like proteins, and any combination or subset thereof. Any one or more complement-related proteins, whether or not expressly mentioned herein, may be excluded from any one or more embodiments.

A “complement-related gene” is a gene that encodes a complement-related protein.

As used herein, the term “complement-mediated disorder” is a disorder in which complement activation (e.g., excessive or inappropriate complement activation) is involved, e.g., as a contributing and/or at least partially causative factor. Complement-mediated disorders of particular interest herein are ones in which one or more complement system biomarkers, e.g., one or more genetic markers, is known to be associated with likelihood of developing or having the disease, such that results of an assay of such complement system biomarker(s) are indicative of the likelihood that a subject who does not have the disease will develop the disease or of the likelihood that a subject has the disease. In particular, complement-mediated disorders of interest herein include age-related macular degeneration, atypical hemolytic uremic syndrome (aHUS), and membranoproliferative glomerulonephritis (MPGN).

As used herein, the term “correlates”, “correlates with”, “correlation”, and like terms, refer to a statistical association between two or more things, such as events, characteristics, outcomes, numbers, data sets, etc., which may be referred to as “variables”. It will be understand that the things may be of different types. Often the variables are expressed as numbers (e.g., measurements, values, likelihood, risk), wherein a positive correlation means that as one variable increases, the other also increases, and a negative correlation (also called anticorrelation) means that as one variable increases, the other variable decreases. Unless otherwise indicated, the terms “correlates”, “correlates with”, “correlation”, and the like as used herein refer to a positive correlation.

As used herein, the term “immune system cell” refers to any of a variety of cells that play a role in the immune response Immune system cells include lymphocytes (T cells, B cells, natural killer (NK) cells); dendritic cells, monocytes, macrophages, eosinophils, mast cells, basophils, and neutrophils. T cells encompass a number of different functional classes that play different roles in the immune response. Different functional classes may be distinguished based on cell surface markers and other properties. Most T cells express an alpha beta (4) T cell receptor (TCR) through which the cell is able to recognize a specific antigen in the context of an appropriate major histocompatibility complex (MHC) molecule, though a minor subset expresses the γδ TCR. Cytotoxic T cells (CTLs) are typically positive for the cell surface marker CD8, which serves as a co-receptor for the TCR in recognition of MHC Class I molecules on the surface of target cells during antigen-specific T cell activation and/or responses. CTLs and NK cells play important roles by eliminating infected host cells and tumor cells through a variety of mechanisms including the release of cytotoxic substances. Helper T cells are typically positive for the cell surface marker CD4, which serves as a co-receptor for the TCR in recognition of MHC Class II molecules on the surface of APCs during antigen-specific T cell activation. Helper T cells promote the activity of other immune system cells (i.e., provide “help”) by, among other things, releasing cytokines that have a variety of effects such as enhancing survival, proliferation, and/or differentiation. Natural killer cells have the ability to recognize and kill (e.g., by causing lysis or apoptosis) cancerous, stressed, or infected cells without requiring antigen-specific activation by presentation of antigen in the context of MHC. Instead, their activation is regulated by a balance of the activity of activating receptors and inhibitory receptors and cytokines. NK cells typically lack cell surface receptors that are highly specific for a particular antigen and are able to react rapidly without prior exposure to the antigen. As used herein, “effector cells” refers to the activated immune system cells that defend the body in an immune response. Effector T cells include cytotoxic T cells and helper T cells, which carry out cell-mediated responses. Effector B cells are called plasma cells and secrete antibodies. Effector cells also include effector NK cells. Regulatory T cells (Tregs) are a subset of CD4⁺ T cells whose normal roles are to suppress immune responses and maintain self-tolerance. The transcription factors FoxP3 and STATS play important roles in their development. Tregs are often CD4⁺CD25⁺ FoxP3⁺ and may be identified based on a cell surface marker expression pattern of CD4⁺CD25⁺CD127^{lo}. Tregs may also be characterized by expression of CTLA4 and GITR. Tregs may suppress the activity of other immune system cell subsets by a variety of mechanisms such as secretion of immunosuppressive cytokines and via cell-cell contact. They may inhibit immune responses at multiple steps, e.g., at the induction of activation (e.g., by inhibiting the ability of APCs to stimulate T cells) and during effector phases. Tregs are often found in tumors, and increased numbers of Tregs has been associated with a worse prognosis in various cancer types. Where it is intended herein to refer to a T cell that is a Treg, the T cell will be identified as such. Thus, unless expressly indicated a T cell, as used herein, is not a Treg cell. An antigen-presenting cell (APC) is a cell that can process and display antigens in association with major histocompatibility complex (MHC) molecules on its surface. T cells may recognize these complexes using their T cell receptors (TCRs). APCs may also display other molecules (costimulatory proteins) that are required for activating naïve T cells. APCs that express MHC class II molecules include dendritic cells, macrophages, and B cells and may be referred to as professional APCs. Dendritic cells (DCs) are white blood cells that occur in most tissues of the body, particularly epithelial tissues. DCs serve as a link between peripheral tissues and lymphoid organs Immature DCs sample the surrounding environment and take up antigenic substances such as pathogen components or tumor antigens. They undergo maturation and migrate to lymph nodes or spleen, where they display fragments of processed antigens at their cell surface using MHC Class 11 (MHCII) complexes. As part of the maturation process, DCs upregulate cell-surface molecules that act as co-stimulators in T cell activation, such as CD80 (B7-1), CD86 (B7-2), and/or CD40. DCs activate helper T cells by presenting them with antigens in the context of MHCII complexes, together with non-antigen specific co-stimulators. DCs and various other APCs have the capacity to activate cytotoxic T cells and B cells through presentation of MHC Class I (MHCI)-peptide complexes (cross-presentation) and costimulators.

“Linked”, as used herein with respect to two or more moieties, means that the moieities are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linking moiety” or “linking portion”, the linkage between the two linked moieties is indirect, and typically each of the linked moieties is covalently bonded to the linking moiety. Two moieties may be linked using a “linker”. A linker can be any suitable moiety that reacts with the entities to be linked within a reasonable period of time, under conditions consistent with stability of the entities (portions of which may be protected as appropriate, depending upon the conditions), and in sufficient amount, to produce a reasonable yield. Typically the linker will contain at least two functional groups, one of which reacts with a first entity and the other of which reacts with a second entity. It will be appreciated that after the linker has reacted with the entities to be linked, the term “linker” may refer to the part of the resulting structure that originated from the linker, or at least the portion that does not include the reacted functional groups. A linking moiety may comprise a portion that does not participate in a bond with the entities being linked, and whose main purpose may be to spatially separate the entities from each other. Such portion may be referred to as a “spacer”.

“Polypeptide”, as used herein, refers to a polymer of amino acids, optionally including one or more amino acid analogs. A protein is a molecule composed of one or more polypeptides. A peptide is a relatively short polypeptide, typically between about 2 and 60 amino acids in length, e.g., between 8 and 40 amino acids in length. The terms “protein”, “polypeptide”, and “peptide” may be used interchangeably. Polypeptides used herein may contain amino acids such as those that are naturally found in proteins, amino acids that are not naturally found in proteins, and/or amino acid analogs that are not amino acids. As used herein, an “analog” of an amino acid may be a different amino acid that structurally resembles the amino acid or a compound other than an amino acid that structurally resembles the amino acid. A large number of art-recognized analogs of the 20 amino acids commonly found in proteins (the “standard” amino acids) are known. One or more of the amino acids in a polypeptide may be modified, for example, by the addition of a chemical entity such as a carbohydrate group, a phosphate group, a farnesyl group, an isofamesyl group, a fatty acid group, a linker for conjugation, functionalization, or other modification, etc. Certain non-limiting suitable analogs and modifications are described in WO2004026328 and/or below. The polypeptide may be acetylated, e.g., at the N-terminus and/or amidated, e.g., at the C-terminus.

In general, polypeptides may be obtained or produced using any suitable method known in the art. For example, polypeptides may be isolated from natural sources, produced in vitro or in vivo using recombinant DNA technology in suitable expression systems (e.g., by recombinant host cells or transgenic non-human animals or plants), synthesized through chemical means such as solid phase peptide synthesis and/or using methods involving chemical ligation of synthesized peptides (see, e.g., Kent, S., J Pept Sci., 9(9):574-93, 2003 and U.S. Pub. No. 20040115774), or a combination of these. One of ordinary skill in the art would readily select appropriate method(s). A polypeptide may comprise a tag, e.g., an epitope tag, which tag may facilitate purification and/or detection of the polypeptide. Exemplary tags include, e.g., 6XHis, HA, Myc, SNUT, FLAG, TAP, etc. In some embodiments, a tag is cleavable, e.g., the tag comprises a recognition site for cleavage by a protease, or is separated from a portion complement inhibiting portion of the polypeptide by a linking portion that comprises a recognition site for cleavage by a protease. For example, a TEV protease cleavage site can be used.

“As used herein, the term “purified” refers to agents that have been separated from most of the components with which they are associated in nature or when originally generated or with which they were associated prior to purification. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate. Any of the agents or substances described herein may be purified.

“Recombinant host cells”, “host cells”, and other such terms, denote prokaryotic or eukaryotic cells or cell lines that contain an exogenous nucleic acid (typically DNA) such as an expression vector comprising a nucleic acid that encodes a polypeptide of interest. It will be understood that such terms include the descendants of the original cell(s) into which the vector or other nucleic acid has been introduced. Appropriate host cells include any of those routinely used in the art for expressing polynucleotides (e.g., for purposes of producing polypeptide(s) encoded by such polynucleotides) including, for example, prokaryotes, such as E. coli; and eukaryotes, including for example, fungi, such as yeast (e.g., Pichia pastoris); insect cells (e.g., Sf9), plant cells, and animal cells, e.g., mammalian cells such as CHO, R1.1, B-W, L-M, African Green Monkey Kidney cells (e.g. COS-1, COS-7, BSC-1, BSC-40 and BMT-10) and cultured human cells. The exogenous nucleic acid may be stably maintained as an episome such as a plasmid or may at least in part be integrated into the host cell's genome, optionally after being copied or reverse transcribed. Terms such as “host cells”, etc., are also used to refer to cells or cell lines that can be used as recipients for an exogenous nucleic acid, prior to introduction of the nucleic acid. A “recombinant polynucleotide” is a polynucleotide that contains nucleic acid sequences that are not found joined directly to one another in nature. For example, the nucleic acid sequences may occur in different genes or different species or one or more of the sequence(s) may be a variant of a naturally occurring sequence or may at least in part be an artificial sequence that is not homologous to a naturally occurring sequence. A “recombinant polypeptide” is a polypeptide that is produced by transcription and translation of an exogenous nucleic acid by a recombinant host cell or by a cell-free in vitro expression system and/or that contains amino acid sequences that are not found joined directly to one another in nature. In the latter case, the recombinant polypeptide may be referred to as a “chimeric polypeptide”. The amino acid sequences in a chimeric polypeptide may, for example, occur in different genes or in different species or one or more of the sequence(s) may be a variant of a naturally occurring sequence or may at least in part be an artificial sequence that is not homologous to a naturally occurring sequence. It will be understood that a chimeric polypeptide may comprise two or more polypeptides. For example, first and second polypeptides A and B of a chimeric polypeptide may be directly linked (A-B or B-A) or may be separated by a third polypeptide portion C (A-C-B or B-C-A). In some embodiments, portion C represents a polypeptide linker which may, for example, comprise multiple glycine and/or serine residues. In some embodiments, two or more polypeptides may be linked by non-polypeptide linker(s).

“Reactive functional groups” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds, N-hydroxysuccinimide esters, maleimides, sulfhydryls, and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989, and Hermanson, G., Bioconjugate Techniques, 2^{nd} ed., Academic Press, San Diego, 2008).

“Specific binding” generally refers to a physical association between a target molecule (e.g., a polypeptide) or molecular complex and a binding molecule such as an antibody or ligand. The association is typically dependent upon the presence of a particular structural feature of the target such as an antigenic determinant, epitope, binding pocket or cleft, recognized by the binding molecule. For example, if an antibody is specific for epitope A, the presence of a polypeptide containing epitope A or the presence of free unlabeled A in a reaction containing both free labeled A and the binding molecule that binds thereto, will reduce the amount of labeled A that binds to the binding molecule. It is to be understood that specificity need not be absolute but generally refers to the context in which the binding occurs. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies or ligands having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target versus the affinity of the binding molecule for other targets, e.g., competitors. If a binding molecule exhibits a high affinity for a target molecule that it is desired to detect and low affinity for nontarget molecules, the antibody will likely be an acceptable reagent. Once the specificity of a binding molecule is established in one or more contexts, it may be employed in other, preferably similar, contexts without necessarily re-evaluating its specificity. In some embodiments, the affinity (as measured by the equilibrium dissociation constant, Kd) of two molecules (or between a molecule and a complex), e.g., two molecules that exhibit specific binding, is 10⁻³ M or less, e.g., 10⁻⁴ M or less, e.g., 10⁻⁵ M or less, e.g., 10⁻⁶M or less, 10⁻⁷M or less, 10⁻⁸M or less, 10⁻⁹M or less, 10⁻¹⁰ M or less, 10⁻¹¹M or less, 10⁻¹² M or less, e.g., between 10⁻¹³M and 10⁻³ M (or within any range having any two of the afore-mentioned values as endpoints) under the conditions tested, e.g., under physiological conditions (e.g., conditions such as salt concentration, pH, and/or temperature, etc., that reasonably approximate corresponding conditions in vivo), or other conditions of the assay. Binding affinity can be measured using any of a variety of methods known in the art. For example, assays based on isothermal titration calorimetry or surface plasmon resonance (e.g., Biacore® assays) can be used in certain embodiments.

A “specific binding agent” is an agent that exhibits specific binding to a target.

A “subject” is typically a human, a non-human primate, or another mammal such as a cow, horse, dog, cat, rodent (e.g., mouse or rat), or rabbit. It will be appreciated that, at least in embodiments wherein a complement inhibitor is administered, the subject should express at least one complement component that can be inhibited by the particular complement inhibitor used. For example, a complement inhibitor specific for primate complement would typically be administered to a human or non-human primate or an animal model that has been genetically engineered to express human complement component(s). In some embodiments the subject is male. In some embodiments the subject is female. In some embodiments, a human subject is at least 12 years of age. In some embodiments a subject is an adult, e.g., a human at least 18 years of age, e.g., between 18 and 100 years of age. The term “subject in need of treatment for cancer”, means a subject who has cancer, e.g., a subject who has been diagnosed as suffering from a cancer, and includes subjects in whom the presence of cancer is detectable as well as subjects who receive adjuvant therapy, for example after surgical removal of a cancer, in an effort to eradicate any residual cancer cells. A subject may sometimes be referred to herein as a “patient” or “individual”. A subject in need of treatment for cancer may be referred to as a “cancer patient”. Any method described herein may be expressly limited to human subjects. Where methods relate to particular biomarkers known to exist in humans and not known to exist in non-human animals, it will be understood that the subject or sample in which such biomarker is assayed is typically a human subject or sample obtained from a human subject.

“Treating”, as used herein in regard to treating a subject, refers to providing treatment, i.e, providing any type of medical or surgical management of a subject. The treatment can be provided in order to reverse, alleviate, inhibit the progression of, prevent or reduce the likelihood of a disease, or in order to reverse, alleviate, inhibit or prevent the progression of, prevent or reduce the likelihood of one or more symptoms or manifestations of a disease. “Prevent” refers to causing a disease or symptom or manifestation of a disease not to occur for at least a period of time in at least some individuals, e.g., individuals at risk of developing the disease, symptom, or manifestation. Treating may include administering an agent, carrying out a procedure (e.g., a surgical procedure), or both, for the purposes of obtaining an effect. Treating can include administering a compound or composition to the subject following the development of one or more symptoms or manifestations indicative of a disease, e.g., in order to reverse, alleviate, reduce the severity of, and/or inhibit or prevent the progression of the disease and/or to reverse, alleviate, reduce the severity of, and/or inhibit or one or more symptoms or manifestations of the disease. A compound or composition can be administered to a subject who has developed a disease, or is at increased risk of developing the disease relative to a member of the general population, optionally a member who is matched with the subject in terms of age, sex, and/or other demographic variable(s). It will be understood that a compound, composition, or intervention useful to treat a subject may be referred to as a “treatment” or “therapy”.

A “variant” of a particular polypeptide or polynucleotide has one or more alterations (e.g., additions, substitutions, and/or deletions, which may be referred to collectively as “mutations”) with respect to the polypeptide or nucleic acid, which may be referred to as the “original polypeptide” or “original polynucleotide”, respectively. Thus a variant can be shorter or longer than the polypeptide or polynucleotide of which it is a variant. The terms “variant” encompasses “fragments”. A “fragment” is a continuous portion of a polypeptide that is shorter than the original polypeptide. In certain embodiments of the invention a variant polypeptide has significant sequence identity to the original polypeptide over a continuous portion of the variant that comprises at least 50%, preferably at least 60%, at least 70%, at least 80%, at least 90%, at least 95%, or more, of the length of the variant or the length of the polypeptide, (whichever is shorter). In certain embodiments of the invention a variant polypeptide has substantial sequence identity to the original polypeptide over a continuous portion of the variant that comprises at least 50%, preferably at least 60%, at least 70%, at least 80%, at least 90%, at least 95%, or more, of the length of the variant or the length of the polypeptide, (whichever is shorter). In a non-limiting embodiment a variant has at least 80% identity to the original sequence over a continuous portion of the variant that comprises between 90% and 100% of the variant, e.g., over 100% of the length of the variant or the length of the polypeptide, (whichever is shorter). In another non-limiting embodiment a variant has at least 80% identity to the original sequence over a continuous portion of the variant that comprises between 90% and 100% of the variant, e.g., over 100% of the length of the variant or the length of the polypeptide, (whichever is shorter). In specific embodiments the sequence of a variant polypeptide has N amino acid differences with respect to an original sequence, wherein N is any integer between 1 and 10. In other specific embodiments the sequence of a variant polypeptide has N amino acid differences with respect to an original sequence, wherein N is any integer between 1 and 20. An amino acid “difference” refers to a substitution, insertion, or deletion of an amino acid.

In certain embodiments a fragment or variant possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Safi, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32(Web Server issue):W522-5, Jul. 1, 2004). In many embodiments one, more than one, or all biological functions or activities of a variant or fragment is substantially similar to that of the corresponding biological function or activity of the original molecule. In certain embodiments the activity of a variant or fragment may be at least 20%, at least 50%, at least 60%, at least 70%, at least 80%, or at least 90% of the activity of the original molecule, up to approximately 100%, approximately 125%, or approximately 150% of the activity of the original molecule. In certain embodiments an activity of a variant or fragment is such that the amount or concentration of the variant needed to produce an effect is within 0.5 to 5-fold of the amount or concentration of the original molecule needed to produce that effect. The disclosure contemplates use of variants of any of the complement inhibiting polypeptides disclosed herein, wherein the variant inhibits complement sufficiently to be useful in a method described herein. In some embodiments, a variant lacks or has a substantially reduction in a property that may be undesired such as immunogenicity.

As used herein, “alkyl” refers to a saturated straight, branched, or cyclic hydrocarbon having from about 1 to about 22 carbon atoms (and all combinations and subcombinations of ranges and specific numbers of carbon atoms therein), with from about 1 to about 12, or about 1 to about 7 carbon atoms being preferred in certain embodiments of the invention. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

As used herein, “halo” refers to F, Cl, Br or I.

As used herein, “alkanoyl” refers to an optionally substituted straight or branched aliphatic acyclic residue having about 1 to 10 carbon atoms (and all combinations and subcombinations of ranges and specific number of carbon atoms) therein, e.g., from about 1 to 7 carbon atoms which, as will be appreciated, is attached to a terminal C═O group with a single bond (and may also be referred to as an “acyl group”). Alkanoyl groups include, but are not limited to, formyl, acetyl, propionyl, butyryl, isobutyryl, pentanoyl, isopentanoyl, 2-methyl-butyryl, 2,2-dimethoxypropionyl, hexanoyl, heptanoyl, octanoyl, and the like, and for purposes of the present invention a formyl group is considered an alkanoyl group. “Lower alkanoyl” refers to an optionally substituted straight or branched aliphatic acyclic residue having about 1 to about 5 carbon atoms (and all combinations and subcombinations of ranges and specific number of carbon atoms). Such groups include, but are not limited to, formyl, acetyl, propionyl, butyryl, isobutyryl, pentanoyl, isopentanoyl, etc.

As used herein, “aryl” refers to an optionally substituted, mono- or bicyclic aromatic ring system having from about 5 to about 14 carbon atoms (and all combinations and subcombinations of ranges and specific numbers of carbon atoms therein), with from about 6 to about 10 carbons being preferred. Non-limiting examples include, for example, phenyl and naphthyl.

As used herein, “aralkyl” refers to alkyl radicals bearing an aryl substituent and having from about 6 to about 22 carbon atoms (and all combinations and subcombinations of ranges and specific numbers of carbon atoms therein), with from about 6 to about 12 carbon atoms being preferred in certain embodiments. Aralkyl groups can be optionally substituted. Non-limiting examples include, for example, benzyl, naphthylmethyl, diphenylmethyl, triphenylmethyl, phenylethyl, and diphenylethyl.

As used herein, the terms “alkoxy” and “alkoxyl” refer to an optionally substituted alkyl-O-group wherein alkyl is as previously defined. Exemplary alkoxy and alkoxyl groups include methoxy, ethoxy, n-propoxy, i-propoxy, n-butoxy, and heptoxy.

As used herein, “carboxy” refers to a —C(═O)OH group.

As used herein, “alkoxycarbonyl” refers to a —C(═O)O-alkyl group, where alkyl is as previously defined.

As used herein, “aroyl” refers to a —C(═O)-aryl group, wherein aryl is as previously defined. Exemplary aroyl groups include benzoyl and naphthoyl.

The term “cyclic ring system” refers to an aromatic or non-aromatic, partially unsaturated or fully saturated, 3- to 10-membered ring system, which includes single rings of 3 to 8 atoms in size and bi- and tri-cyclic ring systems which may include aromatic 5- or 6-membered aryl or aromatic heterocyclic groups fused to a non-aromatic ring. These heterocyclic rings include those having from 1 to 3 heteroatoms independently selected from the group consisting of oxygen, sulfur, and nitrogen. In certain embodiments, the term heterocyclic refers to a non-aromatic 5-, 6-, or 7-membered ring or a polycyclic group wherein at least one ring atom is a heteroatom selected from the group consisting of O, S, and N, including, but not limited to, a bi- or tri-cyclic group, comprising fused six-membered rings having between one and three heteroatoms independently selected from the group consisting of the oxygen, sulfur, and nitrogen. In some embodiments, “cyclic ring system” refers to a cycloalkyl group which, as used herein, refers to groups having 3 to 10, e.g., 4 to 7 carbon atoms. Cycloalkyls include, but are not limited to cyclopropyl, cyclobutyl, cyclopentyl, cyclohexyl, cycloheptyl and the like, which, is optionally substituted. In some embodiments, “cyclic ring system” refers to a cycloalkenyl or cycloalkynyl moiety, which is optionally substituted.

Typically, substituted chemical moieties include one or more substituents that replace hydrogen. Exemplary substituents include, for example, halo, alkyl, cycloalkyl, aralkyl, aryl, sulfhydryl, hydroxyl (—OH), alkoxyl, cyano (—CN), carboxyl (—COOH), —C(═O)O-alkyl, aminocarbonyl (—C(═O)NH₂), —N-substituted aminocarbonyl (—C(═O)NHR″), CF₃, CF₂CF₃, and the like. In relation to the aforementioned substituents, each moiety R″ can be, independently, any of H, alkyl, cycloalkyl, aryl, or aralkyl, for example.

As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

As used herein, an “aromatic amino acid” is an amino acid that comprises at least one aromatic ring, e.g., it comprises an aryl group.

As used herein, an “aromatic amino acid analog” is an amino acid analog that comprises at least one aromatic ring, e.g., it comprises an aryl group.

### II. Methods and Compositions Relating to Immune Checkpoint Inhibitors and the Complement System

The vertebrate immune system comprises a variety of cell types and molecules whose primary role is to protect the body from disease by identifying and eliminating pathogens and tumor cells. However, excessive or inappropriate immune system activity has the potential to inflict significant damage to host tissues. Accordingly, the vertebrate immune system includes a variety of regulatory mechanisms that constrain immune responses or prevent attack on self cells and tissues from occurring. Through a balance between positive and negative signals, these mechanisms normally ensure adequate responses against pathogens, yet prevent over-activation of immune cells that could result in autoimmunity.

Immune checkpoint pathways are a group of inhibitory pathways whose main function is to limit excessive or inappropriate immune system activity under normal conditions. They play important roles in modulating the level and duration of physiological immune responses, such as those that occur during infections, and maintaining self-tolerance (preventing autoimmunity). Immune checkpoint pathways may be activated in various ways. For example, many immune checkpoint pathways are initiated through inhibitory cell surface receptors expressed by various immune system cells, e.g., T cells and natural killer (NK) cells. The receptors for these ligands are typically cell-bound or soluble proteins or small molecules such as adenosine. Ligand binding to these inhibitory receptors initiates signaling pathways that lead to cellular dysfunction. For example, T cells may enter a state of anergy, exhaustion, or senescence, thus inhibiting their ability to carry out their normal functions. T cell anergy refers to an induced hyporesponsive state with low IL-2 production or incomplete activation that naïve T cells can enter if they encounter antigen in the absence of sufficient co-stimulatory stimulation and/or in the presence of high co-inhibitory stimulation. T cell exhaustion is a state of T cell dysfunction that arises during many chronic infections and cancer. It is characterized by poor effector function, sustained expression of inhibitory receptors, and a transcriptional state distinct from that of functional effector or memory T cells, which prevent optimal control of infections and tumors (Wherry, Nat Immunol. 2011; 12(6):492-9). Exhausted T cells have decreased cytokine expression and effector function and are resistant to reactivation.

An “immune checkpoint protein” is generally understoon in the art to be a protein that functions in an immune checkpoint pathway. Examples of immune checkpoint proteins include inhibitory receptors through which an immune checkpoint pathway is initiated, and their ligands. Examples of immune checkpoint pathways include the cytotoxic T-lymphocyte associated antigen 4 (CTLA4) pathway and the programmed cell death 1 (PD1) pathway, both of which are further discussed below. The term “immune checkpoint molecule” encompasses immune checkpoint proteins as well as small molecules such as adenosine that play a role in immune checkpoint pathways.

While immune checkpoint pathways play beneficial roles by protecting mammalian tissues from damage that might otherwise arise as a consequence of inappropriate or excessive immune response, they can also contribute significantly to the ability of cancers to resist destruction by the immune system (Pardoll, Nature Reviews Cancer, 12: 252-264 (2012)). Cancer cells, nontransformed cells within a tumor, or both, may express ligands that bind to inhibitory receptors on immune system cells within the tumor and activate immune checkpoint pathways in these cells, impairing their ability to mount an effective anti-tumor response. The rate of cell death in tumors is high, and dying cells release adenosine, thus providing another mechanism of immune checkpoint pathway activation in cancer patients.

Immune checkpoint inhibitors are a class of anti-cancer agents that enhance anti-tumor immunity by blocking immune checkpoint pathways (Pardoll, supra). Many of these agents bind to immune checkpoint proteins. Ipilimumab, the first immune checkpoint inhibitor to reach the market as a therapeutic agent, is a monoclonal antibody that binds to CLTA4, an immune checkpoint receptor that downregulates T cell activation. Ipilimumab has been approved in a number of countries as a treatment for metastatic melanoma. Ipilimumab and a number of other immune checkpoint inhibitors are currently being studied in clinical trials in a wide variety of advanced cancers. A number of patients treated with such agents demonstrate tumor regression or prolonged stable disease, and some striking responses have been observed. However, overall, only a limited proportion of patients respond, and a significant number of patients experience adverse effects. Therefore, the identification of biomarkers useful for selecting patients most likely to benefit from treatment with immune checkpoint inhibitors is a high priority. It would be of considerable benefit to be able to predict which patients are likely to respond to immune checkpoint inhibitor therapy. It would also be of great benefit to provide ways to increase the likelihood that a patient will respond to immune checkpoint inhibitor therapy or to avoid or overcome lack of response to such therapy.

In some aspects, the present disclosure provides insights into medically significant connections between immune checkpoint pathways and the complement system. Among other things, the disclosure provides the insight that complement has a significant influence on the efficacy of immune checkpoint inhibitor therapy. For example, according to certain embodiments, the likelihood that a subject in need of treatment for cancer will respond to treatment with an immune checkpoint inhibitor may depend at least in part on the state of the complement system in the subject. According to certain embodiments, the likelihood that a subject in need of treatment for cancer will experience disease progression or recurrence after an initial response to treatment with an immune checkpoint inhibitor may depend at least in part on the state of the complement system in the subject. In some embodiments, the likelihood that a subject in need of treatment for cancer will respond to immune checkpoint inhibitor therapy or the likelihood that the subject will experience disease progression or recurrence after an initial response to immune checkpoint inhibitor therapy is determined based on an assay of a complement system biomarker in the subject or in a sample obtained from the subject.

Complement is traditionally considered as playing a beneficial role in the immune response against cancer. However, it has been observed by others that, under certain conditions, complement can promote tumor growth. While a possible role for complement inhibitors as therapeutic agents for the treatment of cancer has been suggested, it has been unclear whether complement inhibition could provide a therapeutically useful effect as a treatment approach in cancer. In particular, it has been unclear in which contexts, if any, treatment with complement inhibitors could be beneficial in cancer. The present disclosure embodies the insight that complement inhibition for treatment of cancer is of particular use in conjunction with treatment with immune checkpoint inhibitors, particularly in cases where immune checkpoint inhibitor treatment has limited efficacy, e.g., in patients who do not respond to immune checkpoint inhibitor therapy or who relapse after an initial response to such therapy. Without wishing to be bound by any particular theory, Applicants propose that complement may promote resistance to immune checkpoint inhibitor therapy through a variety of mechanisms. For example, complement may participate in a tolerogenic cycle involving T regulatory (Treg) cells and dendritic cells (DCs) that limits the ability of the immune system to eliminate cancers. This cycle may contribute to resistance to checkpoint inhibitor therapy. For example, in the setting of chronic exposure to tumor antigens, a population of Treg cells with up-regulated expression of checkpoint proteins such as CTLA4, PD1 and/or LAG3 may be generated. In a manner that is at least in part complement-dependent, these Treg cells promote the development of tolerogenic DCs, which in turn interact with naive CD4 T cells that recognize cognate MHC peptide, causing these cells to develop into Treg cells and thereby establishing a positive feedback loop. In at least some individuals with cancer, this tolerogenic cycle may limit the efficacy of checkpoint inhibitors. For example, the particular checkpoint proteins expressed by the Tregs may vary and may include one or more whose activity is not blocked by the particular checkpoint inhibitor administered and/or the level of checkpoint protein expression and rate of Treg generation may be too high for effective blockage. It is proposed herein that complement inhibition may disrupt this tolerogenic cycle, allowing the benefit of checkpoint inhibitor treatment to be realized.

Alternately or additionally, and without wishing to be bound by any theory, complement inhibition may inhibit the STAT3 pathway in tumor cells and/or in immune cells and thereby potentiate the beneficial effect of checkpoint inhibitor therapy. STAT3 pathway signaling may limit anti-tumor immunity by, e.g., repressing production of pro-inflammatory cytokines (such as type I interferons), inducing release of factors that inhibit activation of multiple immune cell types, and/or by polarizing helper T-cell responses away from a Th1 phenotype (e.g., towards a Th2 or Th17 phenotype). Th1 cells stimulate cytotoxic T lymphocyte responses and are associated with anti-tumor immunity. It is proposed herein that complement inhibition may alter the ratio of Th1 cells to other T helper cell subsets, shifting the balance towards a more robust Th1 response that can, in the presence of checkpoint inhibitor(s), generate a sustained and effective cytotoxic response towards cancers.

Complement may be activated at high levels within tumors. Without wishing to be bound by any theory, this high level complement activation may itself act as an immune checkpoint and may potentially synergize with other immune checkpoint mechanisms to limit the anti-tumor immune response. For example, complement activation products in the tumor, in nearby lymph nodes, and/or in the bloodstream, may saturate complement receptors on immune cells, potentially leading to a state of immune cell exhaustion or allergy. In the presence of high levels of complement activation, immune checkpoint inhibitors may be rendered ineffective because, e.g., immune cells remain exhausted or anergic. It is proposed herein that complement inhibition may act as a “reset” switch, relieving this inhibition and allowing the beneficial effects of immune checkpoint therapy to be realized.

Yet another way in which complement inhibition may potentiate the effect of immune checkpoint inhibitor therapy may be by limiting the generation of sublytic amounts of the membrane attack complex (MAC). As known in the art, the MAC can kill cells by creating pores in the cell membrane. However, sublytic amounts of MAC on cells (e.g., cancer cells) can cause them to become resistant to amounts of MAC that would otherwise be lethal and also to become resistant to other pore-forming agents, including perforin, the pore forming cytolytic protein found in the granules of cytotoxic T lymphocytes and natural killer cells. Certain cancers are capable of avoiding lysis by MAC by, e.g., upregulating complement regulatory proteins, and may thus gain such cross-protection. In a setting in which cancer cells are resistant to lysis by cytotoxic lymphocytes, even a robust and sustained cytotoxic T cell and/or NK cell response, e.g., promoted by immune checkpoint inhibitor therapy, may not be effective in controlling or eliminating the cancer. Complement inhibition may prevent the generation of additional lysis-resistant cancer cells and/or may restore lysis sensitivity as the amount of MAC decreases over time, thereby allowing the benefit of checkpoint inhibitor treatment to be realized. It should be understood that any one or more of the mechanisms described herein by which complement may limit the effectiveness of immune checkpoint inhibitor therapy and/or promote resistance to immune checkpoint inhibitor therapy, or other mechanisms, may be operative in a given subject,

Some aspects of the disclosure relate to methods of selecting or identifying individuals who are appropriate candidates for therapy with an immune checkpoint inhibitor, e.g., for treatment of cancer. Such individuals include patients that have an increased likelihood of benefiting from administration of an immune checkpoint inhibitor relative to other members of the general population having different characteristic(s). In certain embodiments an appropriate candidate is one who is reasonably likely to benefit from treatment or at least sufficiently likely to benefit as to justify administering the treatment in view of its risks and side effects. An aspect of the disclosure relates to methods of selecting or identifying individuals who are not appropriate candidates for therapy with an immune checkpoint inhibitor, e.g., for treatment of cancer. Such individuals include patients that have a decreased likelihood of benefiting from administration of an immune checkpoint inhibitor relative to other members of the general population having different characteristic(s), or a low or substantially no likelihood of benefiting from such treatment, such that it may be desirable to use a different or additional treatment. In some embodiments, whether a subject is an appropriate candidate for therapy with an immune checkpoint inhibitor is determined based on an assay of a complement system biomarker in the subject or in a sample obtained from the subject.

In some aspects, described herein are methods of determining, for example based on an assay of a complement system biomarker, the likelihood that a subject in need of treatment for cancer will respond to treatment with an immune checkpoint inhibitor and/or of identifying and/or selecting a subject to receive such treatment. The phrase “treatment with an immune checkpoint inhibitor”, also referred to as “immune checkpoint inhibitor treatment”, “therapy with an immune checkpoint inhibitor”, or “immune checkpoint inhibitor therapy”, encompasses embodiments pertaining to treatment with a single immune checkpoint inhibitor and embodiments pertaining to treatment with two or more immune checkpoint inhibitors in combination. In some embodiments immune checkpoint inhibitor treatment comprises inhibiting two or more different immune checkpoint pathways using a single agent or using two or more separate agents.

As used herein, a cancer patient who has been treated is considered to “respond”, have a “response”, or be “responsive” to the treatment if the subject shows evidence of an anti-cancer effect according to an art-accepted set of objective criteria or reasonable modification thereof. It will be understood that these terms may also be used in regard to the cancer. A variety of different objective criteria for assessing the effect of anti-cancer treatments on cancers are known in the art. The World Health Organization (WHO) criteria (Miller, A B, et al., Cancer. 1981; 47(1):207-14) and modified versions thereof, the Response Evaluation Criteria in Solid Tumors (RECIST) (Therasse P, et al. J Natl Cancer Inst 2000; 92:205-16), and revised version thereof (Eisenhauer E A, New response evaluation criteria in solid tumours: revised RECIST guideline (version 1.1). Eur J Cancer. 2009; 45(2):228-47) are sets of objective criteria, based on imaging measurements of the size and number of tumor lesions and detection of new lesions, e.g., from computed tomography (CT), magnetic resonance imaging (MRI), or conventional radiographs. Dimensions of selected lesions (referred to as target lesions) are used to calculate the change in tumor burden between images from different time points. The calculated response is then categorized as complete response (CR), partial response (PR), stable disease (SD), or progressive disease (PD). CR is complete disappearance of tumor (−100%), and PD is an increase of about 20%-25% or greater (depending on the particular criteria) and/or the appearance of new lesions. PR is a significant reduction (of at least about 30%) in size of tumor lesions (without emergence of new lesions) but less than a complete response. SD is in between PR and PD. (See Tables 1 and 2 for details.) These criteria are widely used as a primary endpoint in Phase II trials evaluating the efficacy of anti-cancer agents, e.g., as a surrogate for overall survival. However, anatomic imaging alone using WHO, RECIST, and RECIST 1.1 criteria were designed to detect early effects of cytotoxic agents and have certain limitations, particularly in assessing the activity of newer cancer therapies that stabilize disease. Clinical response patterns in patients treated with immunotherapeutic anti-cancer agents or molecularly targeted anti-cancer agents may extend beyond those of cytotoxic agents and can manifest after an initial increase in tumor burden or the appearance of new lesions. For example, meaningful tumor responses to immune checkpoint inhibitor may occur after a delay, in some cases following WHO- or RECIST-defined PD. Criteria designated immune-related response criteria (irRC) were defined in an attempt to capture additional favorable response patterns observed with immune therapies (Wolchok, J D, et al. (2009) Guidelines for the evaluation of immune therapy activity in solid tumors: immune-related response criteria. Clin. Care Res. 15, 7412-7420.). Four patterns associated with favorable survival were identified, i.e., decreased baseline lesions without new lesions; durable stable disease; initial increase in total tumor burden but eventual response; and a reduction in total tumor burden during or after the appearance of new lesion(s), of which the latter two are distinct from the response patterns considered favorable according to WHO or RECIST criteria. The irRC include criteria for complete response (irCR), partial response (irPR), stable disease (irSD), and progressive disease (irPD). Among other things, the irRC incorporates measurable new lesions into “total tumor burden” and compares this variable to baseline measurements rather than assuming that new lesions necessarily represent progressive disease. In summary, according to the immune-related response criteria, irCR is complete disappearance of all lesions whether measurable or not, and no new lesions; irPR is a decrease in tumor burden ≥50% relative to baseline; irSD is disease not meeting criteria for irCR or irPR, in absence of it progressive disease (irPD); irPD is an increase in tumor burden ≥25% relative to nadir (the minimum recorded tumor burden) (Wolchok, supra). irCR, irPR and irPD require confirmation by a repeat, consecutive assessment at least 4 weeks from the date of first documentation. irCR, irPR, and irSD include all patients with CR, PR, or SD by WHO criteria as well as those patients that shift to these irRC categories from WHO PD. However, some patients who would be classified as having PD according to WHO or RECIST criteria are instead classified as having PR or SD according to the irRC, identifying them as likely to have favorable survival. The irRC are applicable to immune checkpoint inhibitors and other immunotherapeutic agents. One of ordinary skill in the art will appreciate that additional response criteria are known in the art, which take into consideration various factors such as changes in the degree of tumor arterial enhancement and/or tumor density as indicators of tumor viable tissue, with decreased arterial enhancement and decreased tumor density being indicators of reduced viable tumor tissue (e.g., due to tumor necrosis). For example, modified RECIST criteria (mRECIST) take into consideration changes in the degree of tumor arterial enhancement (Lencioni R and Llovet J M. Semin Liver Dis 30: 52-60, 2010). Choi criteria and modified Choi criteria take into consideration decrease in tumor density on CT. Choi H, et al., J Clin Oncol 25: 1753-1759, 2007; Nathan P D, et al. Cancer Biol Ther 9: 15-19, 2010; Smith A D, et al. Am J Roentgenol 194: 157-165, 2010. Such criteria may be particularly useful in certain cancer types and/or with certain classes of therapeutic agents. For example, changes in tumor size can be minimal in tumors such as lymphomas, sarcoma, hepatomas, mesothelioma, and gastrointestinal stromal tumor despite effective treatment. CT tumor density, contrast enhancement, or MRI characteristics appear more informative than size. In certain embodiments functional imaging, e.g., using positron emission tomography (PET) may be used. For example, PET response criteria in solid tumors (PERCIST) may be used, in which the treatment response is evaluated by metabolic changes assessed with (18)F-FDG PET imaging, with decreased uptake of the tracer being indicative of (Wahl R L, et al., J Nucl Med 2009; 50, Suppl 1:122S-50S). It will also be understood that response criteria developed for various specific cancer types such as lymphoma, glioblastoma, and hepatocellular carcinoma, are known in the art.

For purposes of the present disclosure, a cancer patient treated with an immune checkpoint inhibitor as monotherapy or in combination with one or more other active agents (e.g., a complement inhibitor, an additional anti-cancer agent, or both) is considered to “respond”, have a “response”, or be “responsive” to the treatment if the patient has a complete response, partial response, or stable disease according at least to the immune-related response criteria. (The cancer patient may also respond according to RECIST, RECIST 1.1, WHO, and/or other criteria such as those mentioned above.) Likewise, the cancer in such cases is said to “respond”, be “responsive”, or be “sensitive” to the treatment. The cancer patient is considered to “not respond”, not have a “response”, or to be “nonresponsive” to the treatment if the patient has progressive disease according to the immune-related response criteria. (The cancer patient may also not respond according to RECIST, RECIST 1.1, WHO, and/or other criteria such as those mentioned above). Likewise, the cancer in such cases said to “not respond”, or to be “nonresponsive”, “insensitive” or “resistant” to the treatment. (A cancer is also considered to have become resistant to treatment if it initially responds but the patient subsequently exhibits progressive disease in the presence of treatment.) Thus, for example, for methods and products described herein that relate to response to treatment for cancer (e.g., methods of predicting likelihood of response, methods of classifying patients according to predicted response, methods of increasing the likelihood of response) a response is defined as irCR, irPR, or irSD, and lack of response is defined as irPD unless otherwise specified. In certain embodiments any useful response criteria may be specified. The response criteria may have been shown to correlate with a benefit such as increased overall survival or other clinically significant benefit. It will be appreciated that refinements or revisions of existing response criteria that, e.g., encompass additional favorable patterns of clinical activity (e.g., correlating with increased overall survival) applicable to immune checkpoint inhibitors or are otherwise useful may be developed in the future. In certain embodiments any such response criteria may be specified for use in methods described herein.

In some aspects, the disclosure provides a methods of treating a subject in need of treatment for cancer, by exposing the subject to combination therapy with an immune checkpoint inhibitor and a complement inhibitor (i.e., by administering immune checkpoint inhibitor therapy, complement inhibitor therapy, or both to a subject so that the subject receives both).

According to the present disclosure, complement inhibitor therapy may enhance the efficacy of immune checkpoint inhibitor therapy in a variety of different ways. For example, in some embodiments, administration of a complement inhibitor to a subject suffering from a cancer that shows resistance (e.g., is nonresponsive) to treatment with an immune checkpoint inhibitor may overcome such resistance. In some embodiments a cancer that is resistant to an immune checkpoint inhibitor may be rendered sensitive (responsive) to such therapy by treating a subject suffering from the cancer with a complement inhibitor. In some embodiments a cancer that is initially sensitive to treatment with an immune checkpoint inhibitor but becomes resistant to such treatment may be rendered sensitive again by treating a subject suffering from the cancer with a complement inhibitor. In some embodiments coadministration of a complement inhibitor and an immune checkpoint inhibitor may delay or prevent the emergence of resistance to the immune checkpoint inhibitor. In some embodiments coadministration of a complement inhibitor and an immune checkpoint inhibitor may increase the overall response rate relative to the overall response rate if the immune checkpoint inhibitor is administered without coadministering the complement inhibitor. In some embodiments coadministration of a complement inhibitor and an immune checkpoint inhibitor may increase the likelihood that a patient will respond relative to the likelihood that the patient will respond if the immune checkpoint inhibitor is administered without coadministering the complement inhibitor. In some embodiments coadministration of a complement inhibitor and an immune checkpoint inhibitor may increase the complete response rate relative to the complete response rate if the immune checkpoint inhibitor is administered without coadministering the complement inhibitor. In some embodiments coadministration of a complement inhibitor and an immune checkpoint inhibitor may increase the likelihood that a patient will have a complete response relative to the likelihood that the patient will exhibit a complete response if the immune checkpoint inhibitor is administered without coadministering the complement inhibitor. In some embodiments coadministration of a complement inhibitor and an immune checkpoint inhibitor may increase the sum of the complete and partial response rates relative to the sum of the complete and partial response rates if the immune checkpoint inhibitor is administered without coadministering the complement inhibitor. In some embodiments coadministration of a complement inhibitor and an immune checkpoint inhibitor may increase the likelihood that a patient will have a complete or partial response relative to the likelihood that the patient will exhibit a complete or partial response if the immune checkpoint inhibitor is administered without coadministering the complement inhibitor. In some embodiments, the overall response rate (i.e., complete response, partial response, and stable disease), complete response rate, or complete+partial response rate, likelihood of response, likelihood of complete response, or likelihood of complete or partial response may increase by at least a factor of 1.2, 1.5, 2.0, 2.5, or more. In some embodiments, the overall response rate, complete response rate, or complete+partial response rate may increase by at least 10, 20, 30, 40, or 50 percentage points. By way of example, an increase in response rate from 10% of patients to 20% of patients would be an increase by a factor of 2 and an increase of 10 percentage points. In some embodiments, the overall response rate, complete response rate, or complete+partial response rate, likelihood of response, likelihood of complete response, or likelihood of complete or partial response may increase up to about 30%, 40%, 50%, 60%, 70%, 80%, 90%, or more. In some embodiments, the overall response rate, complete response rate, or complete+partial response rate may increase by at least 10, 20, 30, 40, or 50 percentage points, e.g., up to about 30%, 40%, 50%, 60%, 70%, 80%, 90%, or more. In some embodiments, such an increase may be seen in patients overall. In some embodiments, such an increase may be seen in one or more particular subpopulations, e.g., patients who are determined, based on an assay of a complement system biomarker, to be appropriate candidates for treatment with an immune checkpoint inhibitor and a complement inhibitor.

The phrase “treatment with a complement inhibitor”, also referred to as “complement inhibitor treatment”, “therapy with a complement inhibitor”, or “complement inhibitor therapy”, encompasses embodiments pertaining to treatment with a single complement inhibitor and embodiments pertaining to treatment with two or more complement inhibitors. In some embodiments pertaining to treatment with two or more complement inhibitors, the complement inhibitors inhibit different complement components. Unless otherwise indicated, “treatment with an immune checkpoint inhibitor” and like terms refer to treatment with an immune checkpoint inhibitor in the absence of combination treatment with a complement inhibitor. Such treatment may also be referred to as “standard treatment with an immune checkpoint inhibitor”. “Combination treatment with an immune checkpoint inhibitor and a complement inhibitor” refers to treatment of a subject with an immune checkpoint inhibitor and with a complement inhibitor for treating the same disorder (e.g., cancer) and encompasses treatment using a treatment regimen for each agent that is appropriate for treating the disorder and treatment in which administration of the immune checkpoint inhibitor and complement inhibitor is coordinated in some way, e.g., so as to enhance or restore the efficacy of an immune checkpoint inhibitor or reduce the likelihood of resistance or nonresponsiveness to treatment with an immune checkpoint inhibitor. In some embodiments administration of an immune checkpoint inhibitor and a complement inhibitor is coordinated in that the agents are administered according to a predetermined schedule that, for example, may specify a particular time interval, minimum or maximum time interval, or range of appropriate time intervals to be used between administration of the agents.

Combination treatment with an immune checkpoint inhibitor and a complement inhibitor encompasses situations in which administration of the agents is coordinated such that the treatment regimen for the complement inhibitor is selected so as to enhance the efficacy of an immune checkpoint inhibitor or reduce the likelihood of resistance or nonresponsiveness to treatment with an immune checkpoint inhibitor that is also administered to the subject. Combination treatment with an immune checkpoint inhibitor and a complement inhibitor may be presumed to be the case if an immune checkpoint inhibitor and a complement inhibitor are prescribed or administered to a subject by or under direction of the same health care professional, if the immune checkpoint inhibitor and a complement inhibitor are administered to a subject who has not been diagnosed with a complement-mediated disorder, or if the immune checkpoint inhibitor and a complement inhibitor are administered to a subject who has been diagnosed with a complement-mediated disorder that is being effectively treated by one or more agents that are not complement inhibitors or for which no complement inhibitor has been shown to be safe and effective. Combination therapy with an immune checkpoint inhibitor and a complement inhibitor is distinct from a situation in which a complement inhibitor is administered for purposes of treating a complement-mediated disorder and an immune checkpoint inhibitor is administered independently for treating a different disorder, e.g., cancer or an infection. For example, intravitreal administration of a complement inhibitor to treat age-related macular degeneration in a subject who is independently treated with an immune checkpoint inhibitor to treat lung cancer would not be considered combination treatment with an immune checkpoint inhibitor and a complement inhibitor. Where combination treatment with an immune checkpoint inhibitor and a complement inhibitor is intended, the use of a complement inhibitor in the treatment regimen will be mentioned.

In some embodiments combination treatment with an immune checkpoint inhibitor and a complement inhibitor comprises pre-treatment with a complement inhibitor prior to treatment with an immune checkpoint inhibitor. The pretreatment may be used to reduce the likelihood of nonresponse to the immune checkpoint inhibitor. In some embodiments the pretreatment occurs at least once within 5, 10, 20, 30, 45, or 60 days before starting therapy with the immune checkpoint inhibitor or resuming such therapy after discontinuing it. In some embodiments treatment with the complement inhibitor continues after treatment with the immune checkpoint inhibitor has started or resumed.

Combination therapy with an immune checkpoint inhibitor and a complement inhibitor may result in increased immune-mediated destruction of tumors and improve the rate of overall tumor response and duration of response. These effects may contribute to an improvement in overall survival compared to treatment using an immune checkpoint inhibitor alone. A treatment regimen comprising an immune checkpoint inhibitor, one or more additional anti-cancer therapies, and a complement inhibitor may result in an improvement in overall survival compared to treatment using the same immune checkpoint inhibitor and the same one or more additional anti-cancer therapies without the complement inhibitor. Overall survival may be measured as the median survival following the initiation of treatment with the immune checkpoint inhibitor. Overall survival may additionally or alternately be measured as the overall survival rate at, e.g., 1 month, 2 months, 3 months, 4 months, 6 months, 9 months, 12 months (1 year), 18 months, 2 years, 3 years, 4 years, 5 years, etc., following the initiation of treatment with the immune checkpoint inhibitor. In some embodiments, the overall survival rate at one or more of the afore-mentioned time points may be increased by at least 10%, 20%, 30%, 40%, 50%, 60%, 70%, 80%, 90%, 100%, 2.5-fold, 3-fold, 4-fold, 5-fold, or more, in subjects treated with an immune checkpoint inhibitor and a complement inhibitor as compared with subjects treated with the same immune checkpoint inhibitor but not treated with the complement inhibitor. In some embodiments, the overall survival rate at one or more of the afore-mentioned time points may be increased by at least 10%, 20%, 30%, 40%, 50%, 60%, 70%, 80%, 90%, 100%, 2.5-fold, 3-fold, 4-fold, 5-fold, or more in subjects who are treated with an immune checkpoint inhibitor, one or more additional anti-cancer therapies, and a complement inhibitor as compared with subjects treated with the same immune checkpoint inhibitor and same additional anti-cancer therapies but not treated with the complement inhibitor. In some embodiments, the overall median survival may be increased by at least 1 month, 2 months, 3 months, 4 months, 6 months, 9 months, 12 months, 18 months, 2 years, or more, in subjects treated with an immune checkpoint inhibitor and a complement inhibitor as compared with subjects treated with the same immune checkpoint inhibitor but not treated with the complement inhibitor. In some embodiments, the overall survival rate at one or more of the afore-mentioned time points may be increased by at least 1 month, 2 months, 3 months, 4 months, 6 months, 9 months, 12 months, 18 months, 2 years, or more in subjects who arc treated with an immune checkpoint inhibitor, one or more additional anti-cancer therapies, and a complement inhibitor as compared with subjects treated with the same immune checkpoint inhibitor and same additional anti-cancer therapies but not treated with the complement inhibitor.

In some aspects, the disclosure provides the recognition that there is an important, medically significant connection between the state of the complement system in a subject and the likelihood that the subject will respond to treatment with an immune checkpoint inhibitor. According to certain aspects described herein, excessive or inappropriate activity of the complement system correlates with nonresponsiveness (failure to respond) to treatment with an immune checkpoint inhibitor. The state of the subject's complement system may be determined based on an assay of a complement system biomarker. Results of such an assay may be used, for example, to predict the likelihood that a subject will respond to treatment with an immune checkpoint inhibitor, to determine whether a subject is an appropriate candidate for treatment with an immune checkpoint inhibitor, or to select an appropriate agent or combination of agents for treating a subject. Any such methods may comprise treating a subject who is determined to be an appropriate candidate for treatment with an immune checkpoint inhibitor by administration of such an agent.

In some aspects, the disclosure provides the recognition that complement system biomarkers that serve as indicators of the susceptibility of a subject to a complement-mediated disorder can be used as indicators of the likelihood that a subject in need of treatment for cancer will respond to treatment with an immune checkpoint inhibitor. Thus, according to some aspects of the disclosure, complement system biomarkers serve as biomarkers for the likelihood of response to immune checkpoint inhibitor therapy. “Susceptibility to a complement-mediated disorder” refers to (i) the likelihood that a subject who does not have a complement-mediated disorder will develop the disorder, (ii) the likelihood that a subject who has the disorder will develop a severe or rapidly progressive form of the disorder relative to the overall likelihood of developing a severe or rapidly progressive form of the disorder among individuals having the disorder, or (iii) both (i) and (ii). It will be understood that complement system biomarkers that are associated with increased or decreased risk of developing a complement-mediated disorder are often identified by comparing subjects who have the disorder (e.g., subjects who have an advanced form of the disorder) with comparable subjects who do not have the disorder and identifying statistically significant differences between the groups with respect to results of an assay of a complement system biomarker. Thus, in general, the likelihood that a subject who does not have a complement-mediated disorder will develop the disorder, as determined based on an assay of a complement system biomarker, is highly correlated with the likelihood that a subject in whom the assay yields the same results (or who has the same underlying characteristic(s) that the assay measures) actually has the disorder. Accordingly, “susceptibility to a complement-mediated disorder”, as determined based on an assay of a complement system biomarker, encompasses the likelihood that a subject in whom the assay yields the same results (or who has the same underlying characteristic(s) that the assay measures) actually has the disorder.

“Increased susceptibility to a complement-mediated disorder” refers to (i) an increased likelihood of developing the complement-mediated disorder relative to comparable members of the general population or relative to comparable individuals not having one or more characteristic(s) that confer the increased susceptibility, (ii) an increased likelihood of developing a severe or rapidly progressive form of the complement-mediated disorder relative to the average risk among comparable individuals having the disorder or relative to comparable individuals not having one or more characteristic(s) that confer the increased susceptibility, or (iii) both (i) and (ii). “Increased susceptibility to a complement-mediated disorder” also encompasses increased likelihood that a subject actually has the disorder relative to the population as a whole or relative to individuals not having one or more characteristic(s) that are found more frequently in comparable individuals who have the disorder than in subjects who do not have the disorder. Likewise, “decreased susceptibility to a complement-mediated disorder” refers to (i) a decreased likelihood of developing the complement-mediated disorder relative to comparable members of the general population or relative to comparable individuals not having one or more characteristic(s) that confer the decreased susceptibility, (ii) a decreased likelihood of developing a severe or rapidly progressive form of the complement-mediated disorder relative to the average risk among comparable individuals having the disorder or relative to comparable individuals not having one or more characteristic(s) that confer the decreased susceptibility; or (iii) both (i) and (ii). “Decreased susceptibility to a complement-mediated disorder” also encompasses decreased likelihood that a subject actually has the disorder relative to the population as a whole or relative to individuals not having one or more characteristic(s) that are found more frequently in comparable individuals who do have the disorder than in subjects who have the disorder.

A particular characteristic or particular result of an assay is considered to be associated with susceptibility to a complement-mediated disorder if the characteristic or result is (i) found at higher or lower frequency among individuals who have the disorder than among comparable individuals who do not have the disorder, (ii) found at higher or lower frequency among comparable individuals who have a severe or rapidly progressive form of the disorder, or (iii) both (i) and (ii). A particular characteristic or particular result of an assay is considered to be associated with increased susceptibility to a complement-mediated disorder if the characteristic or result is (i) found at higher frequency among individuals who have the disorder than among comparable individuals who do not have the disorder, (ii) found at higher frequency among individuals who have a severe or rapidly progressive form of the disorder, or (iii) both (i) and (ii). A particular characteristic or particular result of an assay is considered to be associated with decreased susceptibility to a complement-mediated disorder if the characteristic or result is (i) found at higher frequency among individuals who do not have the disorder than among comparable individuals who have the disorder, (ii) found at higher frequency among individuals who have a mild or slowly progressive form of the disorder than among individuals who have a severe or rapidly progressive form of the disorder, or both (i) and (ii). “Comparable individuals” are individuals who similar with respect to other risk factor(s) for the disorder (e.g., smoking history, diet) and/or standard demographic parameters such as age (e.g., up to 5 years younger or older), gender, and/or race.

According to certain aspects described herein, a correlation exists between a subject's susceptibility to a complement-mediated disorder, e.g., AMD, as determined based on an assay of a complement system biomarker, and the likelihood that the subject will not respond to treatment with an immune checkpoint inhibitor. In certain embodiments, if results of an assay of a complement system biomarker are indicative of an increased susceptibility to a complement-mediated disorder, e.g., AMD, the subject also has an increased likelihood of not responding to treatment with an immune checkpoint inhibitor. Stated another way, in some embodiments, an anticorrelation exists between a subject's susceptibility to a complement-mediated disorder, e.g., AMD, as determined based on an assay of complement system biomarker, and the likelihood that the subject would respond to treatment with an immune checkpoint inhibitor. For example, in some embodiments, a subject with increased susceptibility to AMD as compared to average risk in the population, as determined based on an assay of a complement system biomarker, would be at increased risk of not responding to treatment with an immune checkpoint inhibitor as compared to an average member of the population. In some embodiments, a subject who with increased susceptibility to AMD as compared to average risk in the population, as determined based on an assay of a complement system biomarker, would be at increased risk of not responding to treatment with an immune checkpoint inhibitor as compared to the risk of not responding that a subject who has decreased or average susceptibility to AMD, as determined based on an assay of the complement system biomarker. In some embodiments, a subject with increased susceptibility to AMD as compared to average susceptibility in the population, as determined based on an assay of the complement system biomarker, would have a decreased likelihood of responding to treatment with an immune checkpoint inhibitor as compared to the likelihood of responding that a subject who has decreased risk or average susceptibility AMD, as determined based on an assay of the complement system biomarker. In some embodiments, a subject with decreased susceptibility to AMD relative to average susceptibility among the population, as determined based on an assay of the complement system biomarker, would be at decreased risk of not responding to treatment with an immune checkpoint inhibitor as compared to average risk in the population or as compared to the risk of nonresponsiveness of a member of the population who has decreased susceptibility to AMD. In some embodiments, a subject with decreased susceptibility to AMD relative to average risk in the population would have an increased likelihood of responding to treatment with an immune checkpoint inhibitor as compared to average risk in population or as compared to a member of the population who has increased susceptibility to AMD. In some embodiments, assay results that correlate with AMD susceptibility also correlate with nonresponsiveness to treatment with an immune checkpoint inhibitor. In some embodiments, assay results that correlate with decreased AMD susceptibility correlate with response to treatment with an immune checkpoint inhibitor. As mentioned above, it should be understood that AMD susceptibility refers to susceptibility to AMD as determined based on a complement system biomarker, disregarding other factors that might contribute to AMD risk. Without wishing to be bound by any theory, when results of an assay of a complement system biomarker that serves as an indicator of a subject' susceptibility to a complement-mediated disorder indicate that the subject has increased susceptibility to a complement-mediated disorder, such results are indicative of a state of the complement system characterized by increased complement activation capacity, increased or inappropriate complement activity, impaired regulation, or a combination thereof, which state is also associated with an increased likelihood of non-responsiveness to treatment with an immune checkpoint inhibitor. In some embodiments increased susceptibility to AMD refers to increased susceptibility to advanced AMD, i.e., wet AMD (neovascular AMD), advanced dry AMD, or both. For purposes of the present disclosure, advanced dry AMD refers to geographic atrophy (GA).

In some aspects, described herein are methods of classifying a cancer patient according to predicted likelihood of responding to treatment with an immune checkpoint inhibitor, wherein the classification is based on an assay of complement system biomarker. In some embodiments, if results of the assay indicate that the cancer patient has increased susceptibility to a complement-mediated disorder, the cancer patient is classified as having a decreased likelihood of responding to treatment with an immune checkpoint inhibitor as compared to the likelihood of responding if results of the assay do not indicate an increased susceptibility to a complement-mediated disorder.

In some aspects, described herein are methods of determining, based on an assay of complement system biomarker, whether a subject in need of treatment for cancer is an appropriate candidate for treatment with an immune checkpoint inhibitor. In some embodiments the method comprises determining, based on an assay of complement system biomarker, that the subject is an appropriate candidate for treatment with an immune checkpoint inhibitor. In some embodiments the method further comprises treating the subject with an immune checkpoint inhibitor. In some embodiments, the subject is determined to be an appropriate candidate for treatment with an immune checkpoint inhibitor if results of the assay of the complement system biomarker do not indicate that the subject has increased susceptibility to a complement-mediated disorder. In some embodiments the complement system biomarker is one that serves as an indicator of the likelihood that a subject has or will develop AMD, and the subject is determined to be an appropriate candidate for treatment with an immune checkpoint inhibitor if results of the assay do not indicate that the subject has an increased susceptibility to AMD. The results may, for example, indicate that the patient has a decreased susceptibility to AMD.

In some aspects, described herein are methods of determining, based on an assay of a complement system biomarker, that a subject in need of treatment for cancer is not an appropriate candidate for standard treatment with an immune checkpoint inhibitor. In some embodiments the method further comprises treating the subject with (i) an immune checkpoint inhibitor and (ii) a complement inhibitor, a second anti-cancer agent, or both. In some embodiments the second anti-cancer agent comprises a second immunostimulatory agent. In some embodiments the complement system biomarker is one that serves as an indicator of susceptibility to a complement-mediated disorder. In some embodiments, the subject is determined not to be an appropriate candidate for standard treatment with an immune checkpoint inhibitor if results of the assay of the complement system biomarker indicate that the subject has an increased susceptibility to a complement-mediated disorder. In some embodiments the complement system biomarker is one that serves as an indicator of a subject's susceptibility to AMD. In some embodiments the subject is determined not to be an appropriate candidate for standard treatment an immune checkpoint inhibitor if results of the assay indicate that the subject has an increased susceptibility to AMD.

In some aspects, described herein are methods of determining, based on an assay of a complement system biomarker, that a subject is an appropriate candidate for combination therapy with an immune checkpoint inhibitor and a complement inhibitor. In some embodiments the method further comprises administering an immune checkpoint inhibitor and a complement inhibitor to the subject. In some embodiments the complement system biomarker is one that serves as an indicator of susceptibility to complement-mediated disorder. In some embodiments the cancer patient is determined to be an appropriate candidate for treatment with an immune checkpoint inhibitor and a complement inhibitor if results of the assay indicate that the cancer patient has an increased susceptibility to a complement-mediated disorder. In some embodiments the complement system biomarker is one that serves as an indicator of the likelihood that a subject has or will develop AMD. In some embodiments the subject is determined to be an appropriate candidate for treatment with an immune checkpoint inhibitor and a complement inhibitor if results of the assay indicate that the subject has an increased susceptibility to AMD.

In some aspects, described herein are methods of determining, based on an assay of a complement system biomarker, that a subject in need of treatment for cancer is not an appropriate candidate for standard treatment with an immune checkpoint inhibitor in the absence of combination treatment with a complement inhibitor. In some embodiments the method further comprises administering an immune checkpoint inhibitor and a complement inhibitor to the subject. In some embodiments the complement system biomarker is one that serves as an indicator of the susceptibility of a subject to a complement-mediated disorder. In some embodiments the subject is determined not to be an appropriate candidate for standard treatment with an immune checkpoint inhibitor in the absence of combination treatment with a complement inhibitor if results of the assay indicate that the subject has an increased susceptibility to a complement-mediated disorder. In some embodiments the complement system biomarker is one that serves as an indicator of susceptibility to AMD. In some embodiments the subject is determined not to be an appropriate candidate for standard treatment with an immune checkpoint inhibitor in the absence of combination treatment with a complement inhibitor if results of the assay indicate that the subject has an increased susceptibility to AMD.

In some aspects, described herein are methods of treating a cancer patient. In some embodiments, a method of treating a cancer patient comprises: (a) determining, based on an assay of a complement system biomarker, that the patient is an appropriate candidate for standard treatment with an immune checkpoint inhibitor; and (b) administering an immune checkpoint inhibitor to the patient. In some embodiments, a method of treating a cancer patient comprises: (a) determining, based on an assay of a complement system biomarker, that the patient is not an appropriate candidate for standard treatment with an immune checkpoint inhibitor; and (b) (i) treating the patient with an immune checkpoint inhibitor and a complement inhibitor or (ii) treating the patient with a second anti-cancer agent in combination with or instead of an immune checkpoint inhibitor.

In some embodiments, a method of treating a cancer patient comprises: (a) determining, based on an assay of a complement system biomarker, that the patient is an appropriate patient for combination therapy with an immune checkpoint inhibitor and a complement inhibitor; and (b) treating the patient with an immune checkpoint inhibitor and a complement inhibitor. In some embodiments the complement system biomarker is one that serves as an indicator of susceptibility to a complement-mediated disorder. In some embodiments the patient is determined not to be an appropriate candidate for treatment with an immune checkpoint inhibitor in the absence of combination treatment with a complement inhibitor if results of the assay indicate that the cancer patient has an increased susceptibility to a complement-mediated disorder. In some embodiments the complement system biomarker is one that serves as an indicator of susceptibility to AMD. In some embodiments the cancer patient is determined not to be an appropriate candidate for treatment with an immune checkpoint inhibitor in the absence of combination treatment with a complement inhibitor if results of the assay indicate that the cancer patient has an increased susceptibility to AMD.

In some embodiments, a method of treating a cancer patient comprises: (a) determining, based on an assay of a complement system biomarker, that the patient has increased susceptibility to AMD; and (b) treating the patient with an immune checkpoint inhibitor and a complement inhibitor. In some embodiments, a method of treating a cancer patient comprises: (a) providing a cancer patient who has been tested with an assay of a complement system biomarker and is classified, based on the assay, as being at increased susceptibility to AMD; and (b) treating the patient with an immune checkpoint inhibitor and a complement inhibitor.

In some embodiments, a method of treating a cancer patient comprises: (a) determining, based on an assay of a complement system biomarker, that the patient does not have increased susceptibility to AMD; and (b) treating the patient with an immune checkpoint inhibitor in the absence of combination treatment with complement inhibitor. In some embodiments, a method of treating a cancer patient comprises: (a) providing a cancer patient who has been tested with an assay of a complement system biomarker and is classified as not having increased susceptibility to AMD based on the assay; and (b) treating the patient with an immune checkpoint inhibitor in the absence of combination therapy with a complement inhibitor.

Certain aspects of the disclosure find use in a variety of contexts in which enhancement of the subject's immune response is desired and immune checkpoint pathways contribute to limiting or reducing a subject's immune response. For example, it may be desirable to enhance the immune response of a subject suffering from an infection. A variety of infections are characterized by a state of immune cell dysfunction, e.g., anergy or exhaustion, which may be mediated at least in part by immune checkpoint pathways. Immune checkpoint inhibitors may be useful in treating such disorders. For example, signaling through PD-1 attenuates T cell antigen receptor signals and inhibits the cytokine production and cytolytic function of T cells, both in cancer and in chronic infections. Blockade of PD-1 or PD-L1 during chronic viral infection can restore T cell function and diminish the viral load. In some aspects, described herein is a method of treating a subject in need of an enhanced immune response comprising treating the subject with an immune checkpoint inhibitor and a complement inhibitor. In some embodiments the subject is one in whom an immune checkpoint pathway is overactive as compared with a normal, healthy subject. In some aspects, described herein is a method of reducing or reversing immune cell dysfunction in a subject in need thereof comprising treating the subject with an immune checkpoint inhibitor and a complement inhibitor. In some embodiments the subject is a cancer patient. In some embodiments the subject has an infection, e.g., chronic infection.

Methods of treating a patient, and methods of classifying, determining likelihood of response to treatment, and/or determining whether a patient is an appropriate candidate for treatment, based on an assay of a complement system biomarker, are described herein mainly in regard to cancer patients, but the disclosure also provides embodiments in which such methods are applied in regard to patients suffering from any disorder in which immune checkpoint pathway inhibition is desired, in which immune response enhancement is desired, and/or in which preventing, reducing, or reversing immune cell dysfunction is desired. For example, in some embodiments the subject may suffer from an infection, i.e., a disease resulting from an infectious agent. The infectious agent may be a virus, bacterium, fungus, or parasite (e.g., a unicellular or multicellular parasite). The term “infectious agent” is used interchangeably with “pathogen” herein. In some embodiments an infection is a persistent infection, which term refers to an infection that is not eliminated from the infected subject, even after the induction of an immune response and/or because the agent inhibits induction of an immune response or the subject has an impaired immune system. A persistent infection may be characterized by the continual presence of the infectious agent, sometimes as a latent infection (during which time viral particles are not being produced and the subject is not exhibiting symptoms of viral infection) with occasional relapses of active infection. In some embodiments an infection is a chronic infection, which is defined herein as (i) an infection that has persisted for 2 months or more, in that the infectious agent remains present (typically the agent is detectable using a suitable assay and/or the subject has symptoms or signs of the infection, which may be intermittent) over a period of at least 2 months, e.g., at least 3, or at least 6 months; or (ii) an infection of a type that has a tendency to persist for 2 months or more in a subject with a normal immune system in the absence of treatment. In some embodiments the infection, e.g., chronic infection, is with human immunodeficiency virus, hepatitis C virus, hepatitis B virus, and human herpesviruses (herpes simplex viruses 1 and 2, varicella-zoster virus, EBV (Epstein-Barr virus), human cytomegalovirus, human herpesvirus 6, human herpesvirus 7, or Kaposi's sarcoma-associated herpesvirus. In some embodiments the infection, e.g., chronic infection, is with a member of the genus Babesia, Cryptosporidium, Leishmania, Plasmodium, Schistosoma, Toxoplasma, or Trypanosoma. In some embodiments the infection, e.g., chronic infection, is a bacterial infection, e.g., infection with Mycobacteria (e.g., M. tuberculosis), Chlamydia trachomatis. In some embodiments the bacterium resides intracellularly in mammalian hosts during at least part of its life cycle. In some embodiments the infection, e.g., chronic infection, is a fungal infection, e.g., infection with a fungus of the genus Aspergillus, Blastomyces, Candida, Coccidioides, Cryptococcus, Epidermophytum, Exserohilum, Fusarium, Histoplasma, Malassezia, Microsporum, Mucor, Paracoccidioides, Penicillium, Pichia, Pneumocystis, Pseudallescheria, Rhizopus, Rhodotorula, Scedosporium, Schizophyllum, Sporothrix, Stachybotrys, Saccharomyces, Trichophyton, or Trichosporon.

A subject who is to be treated or is being treated for an infection, e.g., a chronic or persistent infection, may be one whom a medical practitioner has diagnosed as having such a condition. In some embodiments the subject may be or may have been monitored for the infection and/or for response to treatment. Diagnosis and/or monitoring may be performed by any appropriate means. Diagnosis and/or monitoring may involve, for example, detecting the level of the infectious agent in a biological sample (e.g., a tissue biopsy, blood, urine, CSF, sputum sample), detecting the level of a surrogate marker of the infection in a biological sample, detecting symptoms associated with the infection, or detecting immune system cells (or their products) that are involved in the immune response typical of the infection. In some embodiments a subject may be one in whom an infection has persisted or become chronic despite treatment with a conventional anti-viral, anti-bacterial, anti-fungal, or anti-parasite agent or the infection may be one for which a conventional (e.g., approved) treatment is not available. As used herein, a “conventional” treatment is one that does not include an immune checkpoint inhibitor or a complement inhibitor. In some embodiments a subject with an infection is an immunocompromised subject. For example, the subject may be immunocompromised due to treatment with an immunosuppressive drug, due to HIV infection, or due to a genetic immunodeficiency. In some embodiments the infection is an opportunistic infection. In some embodiments the infectious agent is resistant to one or more conventional treatments. For example, a subject with tuberculosis (TB) may have drug-resistant TB, multi-drug resistant TB, or extensively drug-resistant tuberculosis. In some embodiments the subject may have sepsis or be at risk of sepsis.

In the context of treatment for an infection, a “response” may be control (e.g., e.g., substantial suppression of replication of the pathogen and substantial improvement in any symptoms of infection) or, preferably, eradication of the infectious agent.

It will be understood that if the subject suffers from an infection, the subject may additionally be treated with one or more agents appropriate for treating the infection. For example, an appropriate anti-viral, anti-bacterial, anti-fungal, or anti-parasite agent may be used, depending on the particular pathogen. It will also be understood that a subject who is treated or is to be treated with a particular compound may also be treated with one or more agents that reduce the severity and/or likelihood of adverse effects due to the compound. For example, immunosuppressants (or hormone-replacement therapy for endocrinopathies) may be administered to a subject who experiences treatment-related adverse events due to an immune checkpoint inhibitor.

In some embodiments of any of the methods, the step of determining, classifying, or correlating may be based on assays of a single complement system biomarker, 2 or more complement system biomarkers, e.g., 2, 3, 4, or 5, between 5 and 10, between 10 and 20, between 20 and 30, between 30 and 50, or more. Some or all of the biomarkers may be of the same type, e.g., they may all be genetic markers, or a combination of different types of biomarkers may be used, e.g., a genetic marker together with a protein biomarker. Any assay(s) appropriate for detecting, measuring, or otherwise characterizing the particular biomarker(s).

In some embodiments, methods of treating a patient, and methods of classifying, determining likelihood of response to treatment, and/or determining whether a patient (e.g., a cancer patient) is an appropriate candidate for treatment, based on an assay of a complement system biomarker, may be used in combination with other methods that relate to classifying, determining likelihood of response to treatment, and/or determining whether a patient (e.g., a cancer patient) is an appropriate candidate for treatment with an immune checkpoint inhibitor. Such methods may comprise, e.g., measuring the level of an immune checkpoint protein (e.g., PD1 or PD-L1) in a cancer or measuring any biomarker associated with response to immune checkpoint inhibitor treatment.

Methods described herein may, in general, be used with regard to any type of cancer. In certain embodiments the cancer is breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma; pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. It will be appreciated that a variety of different tumor types can arise in certain organs, which may differ with regard to, e.g., clinical and/or pathological features and/or molecular markers. Tumors arising in a variety of different organs are described in the WHO Classification of Tumours series, 4^{th} ed, or 3^{rd} ed (Pathology and Genetics of Tumours series), by the International Agency for Research on Cancer (IARC), WHO Press, Geneva, Switzerland, all volumes of which are incorporated herein by reference. Extensive information regarding different types of cancer and their diagnosis and treatment may be found in DeVita, V T, et al., DeVita, Hellman, and Rosenberg's Cancer: Principles and Practice of Oncology (Cancer: Principles & Practice, Lippincott, Williams, and Wilkins, 9^{th} ed (2011). A subject who is to be treated or is being treated for a cancer may be one whom a medical practitioner has diagnosed as having such a condition. In some embodiments the subject may be or may have been monitored for the cancer and/or for response to treatment. Diagnosis and/or monitoring may be performed by any appropriate means. Diagnosis and/or monitoring may involve, for example, detecting a mass on physical examination, by imaging (e.g., X-ray, CT scan, MRI scan, PET scan, ultrasound), histopathological examination of a biological sample or other means of detecting cancer cells or cancer cell products (e.g., tumor antigens), detecting symptoms associated with cancer. In some embodiments the patient may have exhibited progressive disease or recurrence despite treatment with one or more conventional anti-cancer agents, radiotherapy, or combination thereof. In some embodiments the patient may have exhibited progressive disease or recurrence despite treatment with one or more molecularly targeted anti-cancer agents and/or radiotherapy.

In certain embodiments the cancer type is one for treatment of which an immune checkpoint inhibitor has been tested in at least one Phase I trial and resulted in responses in at least some subjects. In certain embodiments the cancer type is one for treatment of which an immune checkpoint inhibitor has been tested in at least one Phase II trial and resulted in responses in at least some subjects. In certain embodiments the cancer type is one for treatment of which an immune checkpoint inhibitor has been tested in at least one Phase I trial and resulted in responses in at least some subjects. In certain embodiments the cancer type is one for treatment of which an immune checkpoint inhibitor has been approved for use by the US Food & Drug Administration (FDA), the European Medicines Agency (EMA), or both.

In some embodiments the cancer is metastatic, unresectable, or both. In some embodiments the cancer is a Stage III, IIIb, or Stage IV cancer. Cancer stages may be assigned based on the TNM system, described in Sobin L H, Gospodarowicz M K, Wittekind Ch. Eds. TNM Classification of Malignant Tumors, 7th ed. Wiley-Blackwell, Oxford 2009 or in the American Joint Commission on Cancer (AJCC Cancer Staging Manual, Eds. Edge et al., Springer, 7^{th} edition, 2010.

In some embodiments, any of the methods may comprise detecting a complement system biomarker in a sample obtained from the subject, wherein the biomarker is indicative of increased or decreased complement system activity or complement system activation capacity as compared with a control. The control may be a mean value among normal, healthy subjects. In some embodiments the complement system biomarker is detected in a sample comprising a body fluid, e.g., blood or a fraction thereof, e.g., serum or plasma. In some embodiments the sample comprises a tumor sample. In some embodiments, if the biomarker indicates increased complement system activity or complement system activation capacity, the subject has an increased likelihood of non-responsiveness to an immune checkpoint inhibitor as compared to the likelihood of non-responsiveness in the absence of increased complement system activity or complement system activation capacity. In some embodiments the biomarker comprises a complement-related gene or portion thereof or a nucleic acid near a complement related gene. In some embodiments any of the methods may comprise detecting a variation or mutation in or near a complement-related gene, wherein the variation or mutation causes altered expression, stability, or activity of the protein encoded by the gene as compared with the expression, stability, or activity in the absence of the variation or mutation. The alteration in expression, stability, or activity may result in increased or decreased complement system activity or complement system activation capacity as compared with a control. For example, a mutation or variation that causes increased activity of a complement component such as C3 may result in increased complement system activity. A mutation or variation that results in increased activity of a complement regulatory protein such as CFH or CFI may result in decreased complement system activity. In some embodiments, if the biomarker indicates increased complement system activity or complement system activation capacity, the subject is not an appropriate candidate for treatment with an immune checkpoint inhibitor. In some embodiments, if the biomarker indicates increased complement system activity or increased complement system activation capacity, the subject is an appropriate candidate for treatment with an immune checkpoint inhibitor and a complement inhibitor.

In some aspects, described herein is a composition comprising an immune checkpoint inhibitor and a complement inhibitor. In some embodiments the composition is a pharmaceutical composition.

In general, an immune checkpoint inhibitor in any method or composition described herein may comprise any immune checkpoint inhibitor, e.g., any immune checkpoint inhibitor disclosed herein, in various embodiments. For example, in some embodiments, the immune checkpoint inhibitor comprises a CTLA4 pathway inhibitor, e.g., an antibody or other specific binding agent that binds to CTLA4. In some embodiments, the immune checkpoint inhibitor comprises a PD1 pathway inhibitor, e.g., an antibody or other specific binding agent that binds to PD1, PD-L1, or PD-L2. In some embodiments, the immune checkpoint inhibitor inhibits a killer immunoglobulin receptor (KIR) pathway. In some embodiments, the immune checkpoint inhibitor inhibits an immune checkpoint pathway that involves a KIR, LAG3, TIM3, BTLA (B- and T-lymphocyte attenuator), A_{2A}R, or A_{2B}R. For example, in some embodiments the immune checkpoint inhibitor comprises an antibody or other specific binding agent that binds to LAG3, TIM3, BTLA, A_{2A}R, A_{2B}R, or a KIR.

In some embodiments the complement inhibitor comprises any complement inhibitor disclosed herein. For example, in some embodiments the complement inhibitor comprises a compstatin analog. In some aspects, a compstatin analog disclosed in any of the following may be used in a method and/or composition described herein: US Patent App. Pub. Nos. 20100222550, 20070238654, 20080227717, 20120178694, 20140113874, 20070238654; U.S. Ser. No. 14/116,591; U.S. Ser. No. 14/443,143 U.S. Ser. No. 14/443,143; PCT Application No. PCT/US2012/054180 (published as WO/2013/036778); PCT/US2012/037648 (published as WO/2012/155107); PCT/US2013/070424 (published as WO/2014/078734); PCT/US2013/070417 (published as WO/2014/078731).

In some embodiments, the immune checkpoint inhibitor comprises an antibody, aptamer, non-antibody engineered binding protein, dominant negative protein, or other specific binding agent that binds to an immune checkpoint molecule, e.g., an immune checkpoint protein such as CTLA4, PD1, PD-L1, or any other immune checkpoint molecule. All combinations of any genus, subgenus, or species of immune checkpoint inhibitor and any genus, subgenus, or species of complement inhibitor, compositions comprising any such combination, and use of any such combination in any method described herein, are to be considered expressly disclosed herein. Without limiting the foregoing, in some embodiments the immune checkpoint inhibitor comprises a CTLA4 pathway inhibitor, e.g., an antibody that binds to CTLA4, and the complement inhibitor comprises an antibody, compstatin analog, or other specific binding agent that binds to C3. In some embodiments the immune checkpoint inhibitor comprises a CTLA4 pathway inhibitor, e.g., an antibody that binds to CTLA4, and the complement inhibitor comprises an antibody or other specific binding agent that binds to C5 or C5aR. In some embodiments the immune checkpoint inhibitor comprises a CTLA4 pathway inhibitor, e.g., an antibody that binds to CTLA4, and the complement inhibitor comprises an antibody or other specific binding agent that binds to CFB. In some embodiments the immune checkpoint inhibitor comprises a CTLA4 pathway inhibitor, e.g., an antibody that binds to CTLA4, and the complement inhibitor comprises an antibody or other specific binding agent that binds to CFD. In some embodiments the immune checkpoint inhibitor comprises a PD1 pathway inhibitor, e.g., an antibody that binds to PD1, PD-L1, or PD-L2 or a soluble receptor portion that binds to PD-L1 or PD-L2, and the complement inhibitor comprises an antibody, compstatin analog, or other specific binding agent that binds to C3. In some embodiments the immune checkpoint inhibitor comprises a PD1 pathway inhibitor, e.g., an antibody that binds to PD1, PD-L1, or PD-L2 or a soluble receptor portion that binds to PD-L1 or PD-L2, and the complement inhibitor comprises an antibody or other specific binding agent that binds to C5 or C5aR. I n some embodiments the immune checkpoint inhibitor comprises a PD I pathway inhibitor, e.g., an antibody that binds to PD1, PD-L1, or PD-L2 or a soluble receptor portion that binds to PD-L1 or PD-L2, and the complement inhibitor comprises an antibody or other specific binding agent that binds to CFB. In some embodiments the immune checkpoint inhibitor comprises a PD1 pathway inhibitor, e.g., an antibody that binds to PD1, PD-L1, or PD-L2 or a soluble receptor portion that binds to PD-L1 or PD-L2, and the complement inhibitor comprises an antibody or other specific binding agent that binds to CFD. In each case, the antibody or other specific binding agent that binds to a particular molecular target (e.g., an immune checkpoint protein or complement component) may be any of the antibodies or other specific binding agents disclosed herein that bind to that molecular target.

Methods described herein may, in general, be practiced with a variety of complement inhibitors. In some embodiments a complement inhibitor comprises an antibody, aptamer, peptide, polypeptide, or small molecule. In some embodiments the complement inhibitor, e.g., antibody, aptamer, peptide, polypeptide, or small molecule, binds to C3, C5, factor B (CFB), or factor D (CFD). In some embodiments the complement inhibitor, e.g., antibody, aptamer, peptide, polypeptide, or small molecule, binds to C3, C5, or factor D, and inhibits its cleavage. In some embodiments a complement inhibitor comprises a compstatin analog. In some embodiments a complement inhibitor comprises a compstatin analog whose sequence comprises any of SEQ ID NOs: 3-41, e.g., any of SEQ ID NO: 14, 21, 28, 29, 32, 33, 34, or 36. These and other complement inhibitors of use in various embodiments are described further below.

In some embodiments a method comprises monitoring a subject who has responded to an immune checkpoint inhibitor for evidence of resistance to the immune checkpoint inhibitor; detecting evidence of resistance to the immune checkpoint inhibitor; and administering a complement inhibitor to the subject. In some embodiments the method further comprises monitoring the subject for recurrent resistance to the immune checkpoint inhibitor and administering one or more doses of a complement inhibitor to the subject if resistance is detected.

It will be understood that sequences of immune checkpoint proteins, complement-related proteins, and various other naturally occurring proteins, coding sequences, and genes of interest herein are well known in the art and available in public databases such as those available through Entrez at the National Center for Biotechnology Information (www.ncbi.nih.gov) or Universal Protein Resource (www.uniprot.org). Exemplary databases include, e.g., GenBank, RefSeq, Gene, Protein, Nucleotide, UniProtKB/SwissProt, UniProtKB/Trembl, and the like. In general, sequences, e.g., mRNA and polypeptide sequences, in the NCBI Reference Sequence database may be used as reference gene product sequences for a gene of interest. Such sequences may be used, e.g., to produce a polypeptide useful as an antigen or reagent for production, isolation, or characterization of an agent that binds to the gene product (e.g., an antibody that binds to an immune checkpoint protein). The NCBI Gene ID of certain genes of interest are provided herein for the human genes. One of ordinary skill in the art can readily obtain a sequence of any polypeptide, coding sequence (e.g., mRNA, cDNA), or gene. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (see Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (Md.): National Center for Biotechnology Information (US); 2002 (www.ncbi.nlm.nih.gov/bookshelf/br.fcgi?book=handbook&part=ch5, updated February 2011). Polymorphisms in dbSNP encompass single-base nucleotide substitutions (single nucleotide polymorphisms or SNPs), small-scale multi-base deletions or insertions (also called deletion insertion polymorphisms or DIPs), retroposable element insertions, and short tandem repeats. Multiple isoforms of certain proteins may exist, e.g., as a result of alternative RNA splicing or editing. In general, where aspects of this disclosure pertain to a gene or gene product, embodiments pertaining to allelic variants or isoforms are encompassed unless indicated otherwise. Certain embodiments may be directed to particular sequence(s), e.g., particular allele(s) or isoform(s). For example, certain embodiments relate to particular alleles associated with risk of a complement-mediated disorder.

Databases such as dbSNP and those of the International HapMap Project (available at www.hapmap.org) provide a wide variety of information regarding genetica variations, including, e.g., the identity of the nucleotide at each polymorphic position, whether it is a major or minor allele, the sequence surrounding each polymorphic position, chromosome and chromosomal location, gene names and identifiers for SNPs that lie within genes, biological notation, if available, etc. One of ordinary skill in the art can readily identify SNPs in or near particular genes of interest, can readily determine the identity of the possible nucleotides at the polymorphic site and the surrounding sequence, and can readily design probes, primers, etc., to detect such SNPs and/or genotype individuals with respect to such SNPs. In addition, considerable information regarding numerous SNPs is available at the Affymetrix NetAffx™ Analysis Center (www.affymetrix.com/analysis/index.affx). Certain embodiments of the disclosure encompass identification of new SNPs in or linked to genes encoding complement-related proteins and their use in methods described herein.

It will be appreciated that certain of the protein sequences, e.g., in databases, are precursor sequences. The mature form of the protein would lack a secretion signal sequence present in the precursor. It will be appreciated that the sequences described under the respective accession numbers are exemplary and that naturally occurring variants, e.g., allelic variants, are encompassed in various embodiments. Furthermore, it will be appreciated that for purposes of generating a useful binding agent (e.g., an antibody) that binds to a particular polypeptide of interest (e.g., an immune checkpoint molecule or complement component), variant sequences and/or short peptide segments of the polypeptide may be used in certain embodiments.

Certain agents, e.g., immune checkpoint inhibitors and complement inhibitors, of use in various embodiments of the present invention are described further herein. In general, variety of different immune checkpoint inhibitors may be used in various embodiments of the compositions and methods described herein. In general, the immune checkpoint inhibitor may belong to any of various compound classes such as peptides, polypeptides, antibodies (e.g., human or humanized monoclonal antibodies, which may be full size, fragments, single-chain, single domain antibodies, etc.), small molecules, and nucleic acids (e.g., nucleic acid aptamers that bind to a complement component; RNAi agents such as short interfering RNAs that inhibit expression of an immune checkpoint protein by, e.g., causing RNAi-mediated cleavage of mRNA that encodes an immune checkpoint protein or inhibiting translation of such mRNA; antisense oligonucleotides that inhibit expression of an immune checkpoint protein by, e.g., inhibiting translation of such mRNA. In certain embodiments an immune checkpoint inhibitor comprises an antibody or other specific binding agent that binds to an immune checkpoint protein and, e.g., inhibits its activity, e.g., by blocking binding of a ligand of the protein or by blocking binding to a receptor of the protein. In some embodiments immune checkpoint inhibitor comprises a dominant negative form of an immune checkpoint protein or a soluble portion of an immune checkpoint protein that is an inhibitory receptor.

All combinations of the various immune checkpoint inhibitors, complement inhibitors, compound classes, molecular targets, etc.), and dosing parameters (e.g., dosing interval, route of administration, etc.), and disorders disclosed herein are contemplated in various embodiments. It should be understood that other agents that inhibit any of the immune checkpoint pathways described herein or that inhibit complement activation may be used in certain embodiments of the compositions and methods described herein. It will further be understood that variants of the agents may be used. For example, a variant of a protein may comprise a polypeptide that is at least 90%, 91%, 92%, 93%, 94%, 95%, 96%, 97%, 98%, 99%, or more identical to a functional domain of the protein, such as an antigen binding domain of an antibody or a ligand binding domain of a receptor. In some embodiments an antibody that competes for binding to a protein of interest with a particular antibody known in the art, e.g., an antibody that competes with a particular immune checkpoint inhibitor or complement inhibitor, may be used. The competing antibody may be identified by screening or may be engineered based on the sequence of the CDRs of the known antibody and/or based on the identity of the epitope (if known). The competing antibody may bind to the same epitope or a nearby epitope. In some embodiments an antibody of the IgG class may be modified so that it lacks an Fc domain that may activate complement. For example, one or more sites in the Fc domain may be altered to reduce the ability of the Fc domain to activate complement, the Fc domain may be at least in part replaced by at least part of an Fc domain of an IgG4 antibody, a variable region or domain of an IgG1, IgG2, or IgG3 antibody may be grafted to a constant region of an IgG4 antibody, etc. Without wishing to be bound by any theory, avoiding the potential for complement activation by an antibody that serves as an immune checkpoint inhibitor may reduce the likelihood of complement-mediated nonresponsiveness to the antibody. In some embodiments the Fc domain may be altered at one or more sites or at least in part replaced to, e.g., reduce the ability of the Fc domain to elicit antibody-dependent cell-mediated cytotoxicity (ADCC) or bind to the Fc receptor.

### III. Immune Checkpoint Pathways and Immune Checkpoint Inhibitors

CTLA4 Pathway and CTLA4 Pathway Inhibitors

CTLA4 (NCBI Gene ID: 1493) is expressed on T cells, and its principal function is to regulate the extent of the early stages of T cell activation. In general, activation of T cells typically occurs through engagement of the T cell receptor (TCR) and a costimulatory molecule on the T cell. Binding of the T cell receptor to a processed form of its cognate antigen (“cognate antigen” refers to an antigen to which an antigen receptor binds) presented by major histocompatibility complex (MHC) molecules on an antigen presenting cell (APC), e.g., a dendritic cell, provides a first signal for activation. The second signal comes from co-stimulation, in which surface molecules on the APC bind to co-stimulatory receptors on T cells and activate intracellular signaling pathways. CD28 is the most important co-stimulatory receptor for T cell activation and is expressed constitutively by naïve T cells (“naïve” cells are cells that have not encountered cognate antigen). Co-stimulation for these cells comes from the CD80 and CD86 proteins (also called B7-1 and B7-2 respectively), which are expressed by APCs and bind to CD28. In the absence of co-stimulation, T-cell receptor signalling alone can result in anergy. CD28 and CTLA-4 display a different pattern of expression on T-cells: while CD28 is constitutively expressed on the surface of T-cells, CTLA-4 is detectable at low levels in naïve T-cells and more strongly upon T-cell activation. CTLA4 has the same ligands, namely CD80 and CD86, as does CD28, but the affinity of CTLA4 for B7-1 and B7-2 is about 10-fold higher than that of CD28. CTLA4 expression on T cells may counteract the activity of CD28 by competing for binding CD80 and CD86, may actively deliver inhibitory signals to the T cell, or both. Through these and/or other mechanisms, CTLA4 inhibits T cell activation, thus reducing immune responses and anti-tumor immunity. CTLA4 is also expressed by Tregs and promotes their immune suppressive function, further contributing to impairing the immune response to the tumor.

The term “CTLA4 pathway inhibitor” refers to any agent that inhibits the expression or activity of CTLA4 or that otherwise inhibits or interferes with a molecular interaction involving CTLA4 or initiated by the binding of CTLA4 to a natural CTLA4 ligand. CTLA4 pathway inhibitor encompasses any agent that partially or fully blocks, inhibits, neutralizes, prevents or interferes with a biological activity of CTLA4. CTLA4 pathway inhibitor encompasses, for example, any agent that impairs the ability of CTLA4 to cause inhibition of T cell activation, impairs the ability of CTLA4 to enhance Treg proliferation and/or suppressor function, and/or impairs other inhibitory functions of CTLA4 that typically occur following binding of a natural CTLA4 ligand. “CTLA4 inhibitor” refers to any agent that inhibits expression or biological activity of CTLA4. In some embodiments a CTLA4 inhibitor is an agent that specifically binds to CTLA4 and inhibits its activation or activity. In some embodiments a CTLA4 inhibitor is an agent that specifically binds to CTLA4 and blocks interaction of CTLA4 with either or both of the natural CTLA4 ligands (CD80 and CD86). Such an agent may be referred to as a CTLA4 antagonist.

In some embodiments a CTLA4 inhibitor binds to CTLA4 with a Kd of about 10⁻⁶M or less, 10⁻⁷M or less, 10⁻⁸M or less, 10⁻⁹M or less, 10⁻¹° M or less, 10⁻¹¹M or less, 10⁻¹² M or less, e.g., between 10⁻¹³M and 10⁻⁶ M, or within any range having any two of the afore-mentioned values as endpoints. In some embodiments a CTLA4 inhibitor binds to CTLA4 with a Kd of no more than 10-fold that of ipilimumab, when compared using the same assay. In some embodiments a CTLA4 inhibitor binds to CTLA4 with a Kd of about the same as, or less (e.g., up to 10-fold lower, or up to 100-fold lower) than that of ipilimumab, when compared using the same assay. In some embodiments, the IC50 values for inhibition by a CTLA4 inhibitor of CTLA4 binding to CD80 or CD86 is no more than 10-fold greater than that of ipilimumab-mediated inhibition of CTLA4 binding to CD80 or CD86, respectively, when compared using the same assay. In some embodiments, the IC50 values for inhibition by a CTLA4 inhibitor of CTLA4 binding to CD80 or CD86 is about the same or less (e.g., up to 10-fold lower, or up to 100-fold lower) than that of ipilimumab-mediated inhibition of CTLA4 binding to CD80 or CD86, respectively, when compared using the same assay.

In some embodiments a CTLA4 inhibitor is used in an amount sufficient to inhibit expression and/or decrease biological activity of CTLA4 by at least 20%, 30%, 40%, 50%, 60%, 70%, 80%, 90%, 95%, or 100% relative to a suitable control, e.g., between 50% and 75%, 75% and 90%, or 90% and 100%. In some embodiments a CTLA4 pathway inhibitor is used in an amount sufficient to decrease the biological activity of CTLA4 by reducing binding of CTLA4 to CD80, CD86, or both by at least 20%, 30%, 40%, 50%, 60%, 70%, 80%, 90%, 95%, or 100% relative to a suitable control, e.g., between 50% and 75%, 75% and 90%, or 90% and 100% relative to a suitable control. A suitable control in the context of assessing or quantifying the effect of an agent of interest is typically a comparable biological system (e.g., cells or a subject) that has not been exposed to or treated with the agent of interest, e.g., a CTLA4 pathway inhibitor (or has been exposed to or treated with a negligible amount). In some embodiments a biological system may serve as its own control (e.g., the biological system may be assessed before exposure to or treatment with the agent and compared with the state after exposure or treatment has started or finished. In some embodiments a historical control may be used.

CTLA4 inhibitors of use in methods and compositions disclosed herein include antibodies that bind to CTLA4 and other specific binding agents that bind to CTLA4, such as aptamers and non-immunoglobulin engineered binding proteins such as adnectins, affibodies, anticalins, and darpins. In some embodiments such an agent blocks interaction of CTLA4 with either or both of the natural CTLA4 ligands. An anti-CTLA4 antibody may, without limitation, be a monoclonal antibody, e.g., a chimeric monoclonal antibody, a humanized monoclonal antibody, a fully human antibody, a Fab fragment, a single-chain antibody (e.g., an scFv), or a single domain antibody. Various antibodies that bind to CTLA4 and may be used in methods and compositions disclosed herein are known in the art. Suitable CTLA4 inhibitors of use in the methods of the invention, include, without limitation, anti-CTLA4 antibodies, e.g., human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, ipilimumab (also known as MDX-010 and MDX-101), tremelimumab, anti-CTLA4 affibodies, anti-CTLA4 adnectins, anti-CTLA4 domain antibodies, single chain anti-CTLA4 fragments, heavy chain anti-CTLA4 fragments, light chain anti-CTLA4 fragments. For example, U.S. Pat. Nos. 5,811,097; 5,855,887; 5,977,318; 6,051,227; 6,682,736; 6,207,156; 6,984,720; 7,109,003; 7,132,281; 7,605,238 and U.S. Pat. App. Pub. Nos. US20020039581, US2002086014, US20040202650, US 20050201994, US20060165706, US 20110081354, US 20120148597, US20130011405, US20130136749, US20140105914, US20140099325; PCT Publication No. WO 2001/014424, PCT Publication No. WO 2004/035607, European Patent No. EP1212422B1, PCT Publication Nos. WO 01/14424, WO 00/37504, and WO 98/42752 disclose antibodies that bind to CTLA4. In some embodiments an oncolytic adenoviral vector that encodes a monoclonal antibody specific for CTLA4, e.g., a human monoclonal antibody specific for CTLA4, (or the antibodies encoded thereby) may be used. Examples of such vectors are described in US 20130243731.

In some embodiments an anti-CTLA4 antibody binds to CTLA4 and is capable of increasing the response of T cells to antigenic stimulation in vivo yet does not substantially inhibit the binding of a soluble human CTLA4 protein to cells expressing B7-1. In some embodiments an anti-CTLA4 antibody described in any of the afore-mentioned patents, patent applications, or publications may be used in methods and compositions described herein. In some embodiments an anti-CTLA4 antibody that has been tested in at least one Phase I clinical trial in cancer patients may be used. In some embodiments an anti-CTLA4 antibody that has been tested in at least one Phase II clinical trial in cancer patients may be used. In some embodiments an anti-CTLA4 antibody that has advanced at least to a Phase III trial in cancer may be used. Such antibodies include ipilimumab, marketed as Yervoy® (Bristol-Myers Squibb), which is a fully human monoclonal antibody of the IgG1 κ subclass, and tremelimumab (Pfizer, MedImmune) which is a fully human IgG2 subclass monoclonal antibody. In some embodiments an antibody that competes with any of the anti-CTLA4 antibodies mentioned or referred to herein for binding to CTLA4 may be used. For example the antibody may compete with ipilimumab or tremilimumab for binding to CTLA4.

In some embodiments a CTLA4 inhibitor comprises an aptamer that binds to CTLA4. U.S. Patent Application Pub. Nos. US20030054360 and US20060246123 disclose anti-CTLA4 aptamers that may be used in methods and compositions described herein. U.S. Pat. App. Pub. Nos. US20090042785 and US20140051645 disclose anti-CTLA4 anticalins that may be used in methods and compositions described herein. Other CTLA4 inhibitors include RNAi agents, e.g., short interfering RNA, that inhibit expression of CTLA4, and antisense oligonucleotides that inhibit expression of CTLA4.

In some embodiments a cancer patient treated with a CTLA4 pathway inhibitor, e.g., an antibody or other specific binding agent that binds to CTLA4, is in need of treatment for a cancer of any type (see list of cancers elsewhere herein). In certain embodiments the cancer is melanoma (e.g., metastatic melanoma), non-small cell lung carcinoma (NSCLC), small cell lung cancer (SCLC), bladder cancer, prostate cancer, or malignant mesothelioma. In some embodiments the cancer is metastatic, unresectable, or both. In some embodiments the cancer is a Stage III, Mb, or Stage IV cancer, e.g., Stage III, IIIb, or Stage IV melanoma. In some embodiments melanoma is cutaneous melanoma.

In some embodiments, an immune checkpoint inhibitor in any of the compositions or methods described herein that comprise or use an immune checkpoint inhibitor may be a CTLA4 pathway inhibitor. In some embodiments, any of the compositions or methods described herein that comprise or use a CTLA4 pathway inhibitor may comprise or use any CTLA4 pathway inhibitor, e.g., any of the CTLA4 pathway inhibitors described herein.

PD1 Pathway and PD1 Pathway Inhibitors

PD1 (Official symbol: PDCD1 official name: programmed cell death 1; also known as PD1; CD279; SLEB2; NCBI Gene ID: 5133) has two known ligands, PD1 ligand 1 (PD-L1; also known as B7-H1 and CD274; NCBI Gene ID: 29126) and PD-L2 (also known as B7-DC and CD273; NCBI Gene ID: 80380). The PD-1 pathway limits the activity of T cells in peripheral tissues at the time of an inflammatory response to infection and in order to limit autoimmunity. PD1 is a member of the CD28/CTLA4 family that is expressed on activated T cells (Nishimura et al. (1996) Int. Immunol. 8:773). Ligation of PD1 by its ligands mediates an inhibitory signal that results in reduced cytokine production, and reduced T cell survival (Nishimura et al. (1999) Immunity 11:141; Nishimura et al. (2001) Science 291:319; Chemnitz et al. (2004) J. Immunol. 173:945). PD1 expression is induced when T cells become activated. When engaged by one of its ligands, PD1 inhibits kinases that are involved in T cell activation. Like CTLA4, PD1 is highly expressed on Treg cells, and may enhance their proliferation and/or suppressive activity in the presence of a PD1 ligand, which may further suppress immune function. Since many tumors are highly infiltrated with Treg cells, blockade of the PD1 pathway may increase antitumour immune responses by decreasing the number and/or suppressive activity of Treg cells. The term “PD1 pathway inhibitor” refers to any agent that inhibits the expression or activity of PD1, or its natural ligand(s) PD-L1 and/or PD-L2, or that otherwise inhibits or interferes with a molecular interaction involving PD1 or initiated by the binding of PD1 to a natural PD1 ligand. PD1 pathway inhibitor encompasses any agent that partially or fully blocks, inhibits, neutralizes, prevents or interferes with a biological activity of PD1. PD1 pathway inhibitor encompasses any agent that (1) impairs the ability of PD1 to: (i) limit T cell activity (e.g., cytotoxic activity of CD8+ cytotoxic T cells, helper activity of CD4+ helper T cells); (ii) limit NK cell cytotoxic activity; (iii) enhance Treg proliferation and/or suppressor function; or that (2) impairs other inhibitory functions of PD1 that typically occur following binding of a natural PD1 ligand to PD1. “PD1 inhibitor” refers to any agent that inhibits expression or biological activity of PD1. In some embodiments a PD1 inhibitor specifically binds to PD1 and inhibits its activation or activity. In some embodiments a PD1 inhibitor specifically binds to PD1 and blocks interaction of PD1 with PD-L1, PD-L2, or both. “PD-L1 inhibitor” refers to any agent that inhibits expression or biological activity of PD-L1. In some embodiments a PD-L1 inhibitor specifically binds to PD-L1 and inhibits its ability to activate PD1. In some embodiments a PD1 inhibitor specifically binds to PD-L1 and blocks interaction of PD-L1 with PD1. “PD-L2 inhibitor” refers to any agent that inhibits expression or biological activity of PD-L2. In some embodiments a PD-L2 inhibitor specifically binds to PD-L2 and inhibits its ability to activate PD1. In some embodiments a PD-L2 inhibitor specifically binds to PD-L2 and blocks interaction of PD-L2 with PD1.

In some embodiments a PD1 inhibitor, PD-L1 inhibitor, or PD-L2 inhibitor binds to PD1, PD-L1, or PD-L2, respectively, with a Kd of about 10⁻⁶M or less, 10⁻⁷M or less, 10⁻⁸M or less, 10⁻⁹M or less, 10⁻¹⁰ M or less, 10⁻¹¹M or less, 10⁻¹² M or less, e.g., between 10⁻¹³M and 10⁻⁶ M, or within any range having any two of the afore-mentioned values as endpoints. In some embodiments a PD1 inhibitor binds to PD1 with a Kd of no more than 10-fold that of nivolumab, when compared using the same assay. In some embodiments a PD1 inhibitor binds to PD I with a Kd of about the same as, or less (e.g., up to 10-fold lower, or up to 100-fold lower) than that of nivolumab, when compared using the same assay. In some embodiments, the IC50 values for inhibition by a PD1 inhibitor of PD1 binding to PD-L1 or PD-L2 is no more than 10-fold greater than that of nivolumab-mediated inhibition of PD1 binding to PD-L1 or PD-L2, respectively, when compared using the same assay. In some embodiments, the IC50 values for inhibition by a PD1 inhibitor of PD1 binding to PD-L1 or PD-L2 is about the same or less (e.g., up to 10-fold lower, or up to 100-fold lower) than that of nivolumab-mediated inhibition of PD1 binding to PD-L1 or PD-L2, respectively, when compared using the same assay.

In some embodiments a PD1 pathway inhibitor, e.g., a PD1 inhibitor, PD-L1 inhibitor, or PD-L2 inhibitor is used in an amount sufficient to decrease one or more biological activities of PD1 by at least 20%, 30%, 40%, 50%, 60%, 70%, 80%, 90%, 95%, or 100% relative to a suitable control. In some embodiments a PD1 pathway inhibitor decreases the biological activity of PD1 by reducing binding of PD1 to PD-L1, PD-L2, or both by at least 20%, 30%, 40%, 50%, 60%, 70%, 80%, 90%, 95%, or 100% relative to a suitable control. PD1 pathway inhibition, e.g., PD1 blockade, can be accomplished by a variety of mechanisms using any of a variety of agents, including, e.g., with antibodies or other agents that bind PD1 or its ligand(s), PD-L1 and/or PD-L2. In some embodiments the PD1 pathway inhibitor is a PD1 inhibitor, wherein the PD1 inhibitor binds to PD1. For example, in some embodiments the PD1 inhibitor comprises an anti-PD1 antibody. In some embodiments the anti-PD1 antibody is nivolumab (a fully human IgG4 monoclonal antibody also known as BMS-936558 or MDX1106 or ONO) or pembrolizumab (a humanized (from mouse) monoclonal antibody also known as lambrolizumab or MK-3475). In some embodiments the PD1 inhibitor is pidilizumab (also known as CT-011, a humanized antibody that binds PD1). In some embodiments the PD1 inhibitor is MEDI0680, an anti-PD1 monoclonal antibody. In some embodiments the PD1 pathway inhibitor is a PD-L1 inhibitor, wherein the PD-L1 inhibitor binds to PD-L1. For example, in some embodiments the PD-L1 inhibitor is an anti-PD-L1 antibody. In some embodiments the PD1 inhibitor is AMP-224, a fusion protein of PD-L2 and an antibody Fc portion to competitively block PD1. In some embodiments the PD-L1 inhibitor is BMS-936559 (an IgG4 PD-L1 mAb). In some embodiments the PD-L1 inhibitor is MPDL3280A (also called RG7446, a human IgG1-kappa anti-PD-L1 monoclonal antibody that has a single amino acid substitution in its Fc region that normally docks with Fc receptors present on circulating immune cells) or MEDI4736, which is another IgG1-kappa PD-L1 inhibitor. Similar to MPDL3280A, it also has directed mutations in the Fc region that prevent binding to C1q and the Fc gamma receptor. MSB0010718C is another human antibody that is a PD-L1 antagonist. Examples of PD-1, PD-L1, and/or PD-L2 inhibitors that may be used in certain embodiments of methods and/or compositions described herein are described in: U.S. Pat. Nos. 7,488,802; 7,943,743; 8,008,449; 8,168,757; 8,217,149, US Patent Application Pub. Nos. US20130034559; US20140178370; US 20150203580; US 20150203579; US 20150210769; PCT Published Patent Application Nos: WO03042402, WO2008156712, WO2010089411, WO2010036959, WO2011066342, WO2011159877, WO2011082400, and WO2011161699.

In some embodiments a PD1 pathway inhibitor comprises an aptamer that binds to PD1, PD-L1, or PD-L2. Other PD1 pathway inhibitors include RNAi agents, e.g., short interfering RNA, that inhibit expression of PD1, PD-L1, or PD-L2, and antisense oligonucleotides that inhibit expression of PD1, PD-L1, or PD-L2.

Other Immune Checkpoint Pathways and Immune Checkpoint Pathway Inhibitors

Other immune-checkpoint inhibitors of use in certain embodiments include lymphocyte activation gene-3 (LAG3) inhibitors, such as IMP321, a soluble LAG3-Ig fusion protein (Brignone et al., 2007, J. Immunol. 179:4202-4211) or anti-LAG3 monoclonal antibody (BMS-986016).

Other immune-checkpoint inhibitors of use in certain embodiments include B7-H3 and B7-H4 inhibitors, e.g., anti-B7-H3 or anti-B7-H4 antibodies or aptamers. For example, the anti-B7-H3 antibody MGA271 (Loo et al., 2012, Clin. Cancer Res. July 15 (18) 3834) may be used.

Other immune checkpoint pathway inhibitors of use in certain embodiments include TIM3 (T-cell immunoglobulin domain and mucin domain 3) inhibitors (see, e.g., Fourcade et al., 2010, J. Exp. Med. 207:2175-86 and Sakuishi et al., 2010, J. Exp. Med. 207:2187-94). Such agents include, e.g., specific binding agents that bind to TIM3 or to its ligand, galectin 9 (Lgals9). Certain useful agents arc described in US 20150218274.

Other immune checkpoint inhibitors of use in certain embodiments include agents, e.g., antibodies or aptamers, that bind to and inhibit activity of a killer immunoglobulin receptor (KIR) on NK cells that downregulates NK cytotoxic activity. Examples of KIRs include, e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor. For example, lirilumab (BMS-986015, IPH2102) is a human monoclonal antibody of the IgG4 isotype of use for the treatment of cancer that bind to KIR2DL1/2. IPH2101 (also called 1-7F9) is a human IgG4 mAb against common inhibitory KIR2DL-1, KIR2DL-2, and KIR2DL-3 that augments natural killer-mediated killing of tumor cells (Romagne F, et al., Blood 2009; 114(13):2667-2677). Anti-KIR antibodies of use in certain embodiments are disclosed in US Patent Application Publication Nos. US20090035305, US 20090081240; US20130287770, US20120208237, PCT Application Publications WO/2006/003179, WO/2006/072626, WO2012071411.

Immune checkpoint inhibitors of use in certain embodiments include agents that inhibit the BTLA pathway. BTLA is a lymphocyte inhibitory protein with similarities to CTLA4 and PD1. BTLA activation inhibits the function of human CD8+ cancer-specific T cells. Immune checkpoint inhibitors that inhibit the BTLA pathway include, e.g., antibodies or other specific binding agents that bind to BTLA and inhibit the interaction of BTLA with HVEM (herpesvirus entry mediator).

Adenosine is known to exert immunosuppressive effects. In some embodiments an immune checkpoint inhibitor comprises an adenosine pathway inhibitor. Extracellular adenosine is generated from adenosine monophosphate (AMP) by the ectoenzyme CD73 and binds to four known cell surface receptors (Al, A2A, A2B, and A3) that are expressed on multiple immune cell subsets including T cells and NK cells. The A_{2A} and A_{2B} receptor subtypes are responsible for adenosine's immunosuppressive effects. CD73 is overexpressed in a number of cancer types. Both host and tumor-expressed CD73 have been shown to be important in the suppression of antitumor T-cell responses Immune checkpoint inhibitors of use in certain embodiments as adenosine pathway inhibitors include A_{2A}R antagonists, A_{2B}R antagonists, and anti-CD73 agents. Examples of A_{2A}R antagonists include istradefylline, SCH-58261, SCH-442,416, ZM-241,365, ZM-241,385, and analogs thereof. Examples of A_{2B}R antagonists include CVT-6883, MRS-1706, MRS-1754, PSB-603, PSB-0788, and PSB-1115. See Shook B C & Jackson P F, ACS Chem Neurosci. 2011; 2(10):555-67 and Müller CE1, Jacobson K A. Biochim Biophys Acta. 2011; 1808(5):1290-308, and references in either of the foregoing, which are incorporated herein by reference, for additional examples of A_{2A}R and A_{2B}R antagonists. Examples of anti-CD73 agents include anti-CD73 antibodies and other specific binding agents that bind to CD73 and adenosine analogs such as 5′-(α,β-methylene) diphosphate (APCP). Other are described in Antonioli, L, et al., Nature Reviews Cancer 13, 842-857 (2013) for further discussion of the adenosine pathway and adenosine pathway inhibitors in cancer.

In some embodiments, an immune checkpoint inhibitor inhibits indoleamine 2,3-dioxygenase (IDO), an immunoregulatory enzyme that suppresses T-cell responses and promotes immune tolerance. IDO catabolizes tryptophan and is believed to help tumor cells escape the immune system at least in part by depleting Trp in the tumor microenvironment. IDO helps create a tolerogenic milieu within the tumor and the associated tumor-draining lymph nodes. IDO directly suppresses the proliferation and differentiation of effector T cells, and markedly enhances the suppressor activity of Tregs. A wide range of human cancers, including prostate, colorectal, pancreatic, cervical, gastric, ovarian, head, lung, renal cell carcinoma, glioblastoma, etc. have been found to overexpress IDO. IDO inhibitors include, e.g., hydroxyamidines such as INCB023843 and INCB024360 (WO 2006122150) and tryptophan analogs such as 1-methyl tryptophan, dextro-1-methyl tryptophan (D-1MT). Other IDO inhibitors are described in US20120277217, US20140315962, and/or US20140323740.

Combinations of Immune Checkpoint Inhibitors

As mentioned above, in some embodiments a patient may be treated with two or more immune checkpoint inhibitors. In some embodiments, the immune checkpoint inhibitors inhibit different immune checkpoint pathways. For example, in some embodiments a first immune checkpoint inhibitor inhibits the PD1 pathway (e.g., is a PD1 inhibitor, PD-L1 inhibitor, of PD-L2 inhibitor), and a second immune checkpoint inhibitor inhibits the CTLA4 pathway. In some embodiments ipilimumab and nivolumab are used. In some embodiments ipilimumab and pembrolizumab are used. In some embodiments ipilimumab and pidilizumab are used. In some embodiments ipilimumab and MEDI0680 are used. In some embodiments (i) tremilimumab and (ii) nivolumab, pembrolizumab, pidilizumab, or MEDI0680 (or any combination of these) are used. In some embodiments (i) ipilimumab and (ii) BMS-936559, MPDL3280A, MEDI4736, or MSB0010718C (or any combination of these) are used. In some embodiments (i) tremilimumab and (ii) BMS-936559, MPDL3280A, MEDI4736, or MSB0010718C (or any combination of these) are used. In some embodiments nivolumab, pembrolizumab, or pidilizumab (or any combination of these) and BMS-936559, MPDL3280A, MEDI4736, or MSB0010718C (or any combination of these) are used. In some embodiments (i) ipilimumab or tremilimumab and (ii) nivolumab, pembrolizumab, or pidilizumab (or any combination of these) and (iii) BMS-936559, MPDL3280A, MEDI4736, or MSB0010718C (or any combination of these) are used.

In some embodiments a first agent inhibits the PD1 pathway and a second agent comprises a TIM3 inhibitor, BTLA pathway inhibitor, KIR inhibitor, LAG3 inhibitor, or adenosine pathway inhibitor. In some embodiments a first agent inhibits the CTLA4 pathway and a second agent comprises a TIM3 inhibitor, BTLA pathway inhibitor, KIR inhibitor, LAG3 inhibitor, or adenosine pathway inhibitor. In some embodiments a first agent comprises an inhibitor of the PD1 pathway, CTLA4 pathway or comprises aTIM3 inhibitor, BTLA pathway inhibitor, KIR inhibitor, LAG3 inhibitor, or adenosine pathway inhibitor, and a second agent comprises an IDO inhibitor.

In some embodiments the immune checkpoint inhibitors inhibit the same immune checkpoint pathway, e.g., both inhibit the PD1 pathway or both inhibit the CTLA4 pathway. In some embodiments the immune checkpoint inhibitors act on different molecular targets within the same immune checkpoint pathway, e.g., one inhibitor acts on a ligand, and a second inhibitor acts on a receptor for the ligand. For example, in some embodiments, (i) nivolumab, pembrolizumab, pidilizumab, MEDI0680, AMP-224 (or any combination of these); and (ii) BMS-936559, MPDL3280A, MEDI4736, or MSB0010718C (or any combination of these) are used.

In some embodiments the immune checkpoint inhibitors act on the same target. In some embodiments nivolumab and pembrolizumab are used. In some embodiments nivolumab and pidilizumab are used. In some embodiments pembrolizumab and pidilizumab are used.

In some embodiments, a combination of immune checkpoint inhibitors comprises no more than 2, 3, 4, or 5 immune checkpoint inhibitors.

In some embodiments, two or more immune checkpoint inhibitors may be provided or administered as part of a multifunctional, e.g., bifunctional, agent. For example, a bispecific, trispecific, or tetraspecific antibody (or other binding agent) capable of binding to two, three, or four distinct immune checkpoint molecules may be used. In some embodiments the multispecific agent may bind to any two or more of the immune checkpoint molecules disclosed herein. In some embodiments a bifunctional agent, e.g., a bifunctional antibody, binds to PD1 and TIM3, to PD-L1 and TIM3, to PD1 and LAG3, to PD-L1 and LAG3, etc. For example, without limitation, bifunctional antibodies against PD1 and TIM3 are disclosed in WO/2011/159877.

### IV. Complement System and Complement Inhibitors

In order to facilitate understanding of the invention, and without intending to limit the invention in any way, this section provides an overview of complement, its pathways of activation, and some of its activities. Further details are found, e.g., in Kuby Immunology, 7^{h} ed., 2013; Paul, W. E., Fundamental Immunology, Lippincott Williams & Wilkins; 7^{th} ed., 2013; and Walport M J., Complement. First of two parts. N Engl J Med., 344(14):1058-66, 2001.

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils.

The alternative pathway is initiated by and amplified at, e.g., microbial surfaces and various complex polysaccharides. In this pathway, hydrolysis of C3 to C3(H2O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by host cell molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on host cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4bp). CD59 is a membrane-bound complement regulatory protein unrelated structurally to the CCPs. Complement regulatory proteins normally serve to limit complement activation that might otherwise occur on cells and tissues of the mammalian, e.g., human host.

Complement Inhibitors

General

A variety of different complement inhibitors may be used in various embodiments of the compositions and methods described herein. In general, the complement inhibitor may belong to any of various compound classes such as peptides, polypeptides, antibodies (e.g., human or humanized monoclonal antibodies, which may be full size, fragments, single-chain, single domain antibodies, etc.), small molecules, and nucleic acids (e.g., nucleic acid aptamers that bind to a complement component; RNAi agents such as short interfering RNAs that inhibit expression of a complement component by, e.g., causing RNAi-mediated cleavage of mRNA that encodes a complement component or inhibiting translation of such mRNA; antisense oligonucleotides that inhibit expression of a complement component by, e.g., inhibiting translation of such mRNA. In certain embodiments a complement inhibitor comprises an antibody or other specific binding agent that binds to a complement component and, e.g., inhibits its activity or cleavage. In certain embodiments a complement inhibitor inhibits an enzymatic activity of a complement protein. The enzymatic activity may be proteolytic activity, such as ability to cleave another complement protein. In some embodiments, a complement inhibitor inhibits cleavage of C3, C4, C5, or factor B. In some embodiments, a complement inhibitor acts on a complement component that lies upstream of C3 in the complement activation cascade. In some embodiments, a complement inhibitor binds to C3. In some embodiments a complement inhibitor that inhibits C3 activation or activity is used. In some embodiments, a complement inhibitor binds to C4. In some embodiments, a complement inhibitor binds to factor B. In some embodiments, a complement inhibitor binds to factor D. In some embodiments, a complement inhibitor binds to C5. In certain embodiments a complement inhibitor that inhibits at least the classical pathway of complement activation is used. In certain embodiments a complement inhibitor that inhibits at least the alternative pathway of complement activation is used. In certain embodiments a complement inhibitor that inhibits both the classical and the alternative pathway is used. In certain embodiments a complement inhibitor that inhibits the classical pathway, the alternative pathway, and the MBL pathway is used. In some embodiments, a complement inhibitor inhibits activation of at least one complement receptor protein. In certain embodiments the complement receptor protein is a receptor for C3 a, e.g., C3aR. In certain embodiments the complement receptor protein is a receptor for C5a, e.g., C5aR or C5L2. In certain embodiments the complement inhibitor does not bind to C5. In certain embodiments the complement inhibitor does not bind to C5aR, C5a, or C5L2. In certain embodiments the complement inhibitor binds to C3a or C3aR. In certain embodiments the complement inhibitor does not bind to C3a or C3aR.

In some embodiments, a complement inhibitor comprises an antibody that substantially lacks the capacity to activate complement. For example, the antibody may have less than 20%, less than 10%, less than 5%, or less than 1% complement stimulating activity as compared with full length human IgG1. In some embodiments, the antibody comprises a CH2 domain that has reduced ability to bind C1q as compared with human IgG1 CH2 domain. In some embodiments, the antibody contains CH1, CH2, and/or CH3 domains from human IgG4 and/or does not contain CH1, CH2, and/or CH3 domains from human IgG1.

In some embodiments, a complement inhibitor has a molecular weight of between 50 Daltons and 1 kilodalton (kD). In some embodiments, a complement inhibitor has a molecular weight between 1 kD and 2 kD, between 2 kD and 5 kD, between 5 kD and 10 kD, between 10 kD and 20 kD, between 20 kD and 30 kD, between 30 kD and 50 kD, between 50 kD and 100 kD, or between 100 kD and 200 kD.

In some embodiments, a complement inhibitor may be at least in part identical to a naturally occurring complement inhibiting agent or a variant or fragment thereof. A variety of different complement inhibiting polypeptides are produced by viruses (e.g., Poxviruses, Herpesviruses), bacteria (e.g., Staphylococcus), and other microorganisms. Complement inhibiting proteins are produced by various parasites, e.g., ectoparasites, such as ticks. A complement inhibitor can comprise at least a portion of a mammalian complement control or complement regulatory protein or receptor. See Ricklin, D., et al., Nature Biotechnology, 25(11): 1265-75, 2007, for discussion of certain complement inhibitors that are or have been in preclinical or clinical development for various disorders.

In some embodiments a complement inhibitor is used in an amount sufficient to inhibit expression or activity of one or more complement components by at least 20%, 30%, 40%, 50%, 60%, 70%, 80%, 90%, 95%, or 100% relative to a suitable control, e.g., between 50% and 75%, 75% and 90%, or 90% and 100% relative to a suitable control. In some embodiments a complement inhibitor is used in an amount sufficient to inhibit complement activation capacity or complement activation via the classical, alternative, by at least 20%, 30%, 40%, 50%, 60%, 70%, 80%, 90%, 95%, or 100% relative to a suitable control, e.g., between 50% and 75%, 75% and 90%, or 90% and 100% relative to a suitable control.

The following sections further discuss non-limiting exemplary complement inhibitors of use in embodiments of the present invention. Complement inhibitors have been classified in various groups for purposes of convenience. It will be understood that certain complement inhibitors fall into multiple categories.

In some embodiments a complement inhibitor comprises an adnectin, affibody, anticalin, or other type of engineered polypeptide sometimes used in the art in lieu of an antibody, wherein the engineered polypeptide binds to a complement component, e.g., C3, C4, factor B, factor D, or C5.

In some embodiments, a complement inhibitor that binds to substantially the same binding site (e.g., a binding site on a complement component such as C3, C5, factor B, factor D, or an active complement split product) as a complement inhibitor described herein is used. In general, the ability of first and second agents to bind to substantially the same site on a target molecule, such as a complement component or receptor, can be assessed using methods known in the art, such as competition assays, molecular modeling, etc. (See, e.g., discussion of compstatin analog mimetics.) Optionally the first and/or second agent can be labeled with a detectable label, e.g., a radiolabel, fluorescent label, etc. Optionally the target molecule, first agent, or second agent is immobilized on a support, e.g., a slide, filter, chip, beads, etc. In some embodiments, a second antibody that binds to substantially the same binding site as a first antibody comprises one or more CDR(s) that are at least 90%, 91%, 92%, 93%, 94%, 95%, 96%, 97%, 98%, 99%, or 100% identical to CDR(s) of the first antibody.

Compounds that Inhibit C3 Activation or Activity

Compstatin Analogs and Mimetics

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 2 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32 (Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345) and discussion below.

Compstatin analogs may be acetylated or amidated, e.g., at the N-terminus and/or C-terminus. For example, compstatin analogs may be acetylated at the N-terminus and amidated at the C-terminus. Consistent with usage in the art, “compstatin” as used herein, and the activities of compstatin analogs described herein relative to that of compstatin, refer to compstatin amidated at the C-terminus (Mallik, 2005, supra).

Concatamers or multimers of compstatin or a complement inhibiting analog thereof are also of use in the present invention.

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

The activity of a compstatin analog may be expressed in terms of its IC₅₀ (the concentration of the compound that inhibits complement activation by 50%), with a lower IC₅₀ indicating a higher activity as recognized in the art. The activity of a preferred compstatin analog for use in the present invention is at least as great as that of compstatin. It is noted that certain modifications known to reduce or eliminate complement inhibiting activity and may be explicitly excluded from any embodiment of the invention. The IC₅₀ of compstatin has been measured as 12 μM using an alternative pathway-mediated erythrocyte lysis assay (WO2004/026328). It will be appreciated that the precise IC₅₀ value measured for a given compstatin analog will vary with experimental conditions (e.g., the serum concentration used in the assay). Comparative values, e.g., obtained from experiments in which IC₅₀ is determined for multiple different compounds under substantially identical conditions, are of use. In one embodiment, the IC₅₀ of the compstatin analog is no more than the IC₅₀ of compstatin. In certain embodiments of the invention the activity of the compstatin analog is between 2 and 99 times that of compstatin (i.e., the analog has an IC₅₀ that is less than the IC₅₀ of compstatin by a factor of between 2 and 99). For example, the activity may be between 10 and 50 times as great as that of compstatin, or between 50 and 99 times as great as that of compstatin. In certain embodiments of the invention the activity of the compstatin analog is between 99 and 264 times that of compstatin. For example, the activity may be 100, 110, 120, 130, 140, 150, 160, 170, 180, 190, 200, 210, 220, 230, 240, 250, 260, or 264 times as great as that of compstatin. In certain embodiments the activity is between 250 and 300, 300 and 350, 350 and 400, or 400 and 500 times as great as that of compstatin. The invention further contemplates compstatin analogs having activities between 500 and 1000 times that of compstatin, or more, e.g., between 1000 and 2000 times that of compstatin, or more. In certain embodiments the IC₅₀ of the compstatin analog is between about 0.2 μM and about 0.5 μM. In certain embodiments the IC₅₀ of the compstatin analog is between about 0.1 μM and about 0.2 μM. In certain embodiments the IC₅₀ of the compstatin analog is between about 0.05 μM and about 0.1 μM. In certain embodiments the IC₅₀ of the compstatin analog is between about 0.001 μM and about 0.05 μM.

The K_{d} of compstatin binding to C3 can be measured using isothermal titration calorimetry (Katragadda, et al., J. Biol. Chem., 279(53), 54987-54995, 2004). Binding affinity of a variety of compstatin analogs for C3 has been correlated with their activity, with a lower K_{d} indicating a higher binding affinity, as recognized in the art. A linear correlation between binding affinity and activity was shown for certain analogs tested (Katragadda, 2004, supra; Katragadda 2006, supra). In certain embodiments of the invention the compstatin analog binds to C3 with a K_{d} of between 0.1 μM and 1.0 μM, between 0.05 μM and 0.1 μM, between 0.025 μM and 0.05 μM, between 0.015 μM and 0.025 μM, between 0.01 μM and 0.015 μM, or between 0.001 μM and 0.01 μM.

Compounds “designed or identified based on compstatin” include, but are not limited to, compounds that comprise an amino acid chain whose sequence is obtained by (i) modifying the sequence of compstatin (e.g., replacing one or more amino acids of the sequence of compstatin with a different amino acid or amino acid analog, inserting one or more amino acids or amino acid analogs into the sequence of compstatin, or deleting one or more amino acids from the sequence of compstatin); (ii) selection from a phage display peptide library in which one or more amino acids of compstatin is randomized, and optionally further modified according to method (i); or (iii) identified by screening for compounds that compete with compstatin or any analog thereof obtained by methods (i) or (ii) for binding to C3 or a fragment thereof. Many useful compstatin analogs comprise a hydrophobic cluster, a β-turn, and a disulfide bridge.

In certain embodiments of the invention the sequence of the compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments of the invention the amino acid at position 4 is altered. In certain embodiments of the invention the amino acid at position 9 is altered. In certain embodiments of the invention the amino acids at positions 4 and 9 are altered. In certain embodiments of the invention only the amino acids at positions 4 and 9 are altered. In certain embodiments of the invention the amino acid at position 4 or 9 is altered, or in certain embodiments both amino acids 4 and 9 are altered, and in addition up to 2 amino acids located at positions selected from 1, 7, 10, 11, and 13 are altered. In certain embodiments of the invention the amino acids at positions 4, 7, and 9 are altered. In certain embodiments of the invention amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus. In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus. It should be understood that compstatin analogs may have any one or more of the characteristics or features of the various embodiments described herein, and characteristics or features of any embodiment may additionally characterize any other embodiment described herein, unless otherwise stated or evident from the context. In certain embodiments of the invention the sequence of the compstatin analog comprises or consists essentially of a sequence identical to that of compstatin except at positions corresponding to positions 4 and 9 in the sequence of compstatin.

Compstatin and certain compstatin analogs having somewhat greater activity than compstatin contain only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain compstatin analogs having improved activity incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments of the invention, a compstatin analog is designed by replacing one or more L-amino acids in a compstatin analog described elsewhere herein with the corresponding D-amino acid. Such compounds and methods of use thereof are an aspect of the invention. Exemplary non-standard amino acids of use include 2-naphthylalanine (2-NaI), 1-naphthylalanine (1-NaI), 2-indanylglycine carboxylic acid (2-NaI), dihydrotrpytophan (Dht), 4-benzoyl-L-phenylalanine (Bpa), 2-a-aminobutyric acid (2-Abu), 3-a-aminobutyric acid (3-Abu), 4-a-aminobutyric acid (4-Abu), cyclohexylalanine (Cha), homocyclohexylalanine (hCha), 4-fluoro-L-tryptophan (41W), 5-fluoro-L-tryptophan (51W), 6-fluoro-L-tryptophan (61W), 4-hydroxy-L-tryptophan (4OH-W), 5-hydroxy-L-tryptophan (5OH-W), 6-hydroxy-L-tryptophan (6OH-W), 1-methyl-L-tryptophan (1MeW), 4-methyl-L-tryptophan (4MeW), 5-methyl-L-tryptophan (5MeW), 7-aza-L-tryptophan (7aW), α-methyl-L-tryptophan (aMeW), β-methyl-L-tryptophan (βMeW), N-methyl-L-tryptophan (NMeW), ornithine (orn), citrulline, norleucine, γ-glutamic acid, etc.

In certain embodiments of the invention the compstatin analog comprises one or more Trp analogs (e.g., at position 4 and/or 7 relative to the sequence of compstatin). Exemplary Trp analogs are mentioned above. See also Beene, et. al. Biochemistry 41: 10262-10269, 2002 (describing, inter alia, singly- and multiply-halogenated Trp analogs); Babitzke & Yanofsky, J. Biol. Chem. 270: 12452-12456, 1995 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Other Trp analogs include variants that are substituted (e.g., by a methyl group) at the α or β carbon and, optionally, also at one or more positions of the indole ring Amino acids comprising two or more aromatic rings, including substituted, unsubstituted, or alternatively substituted variants thereof, are of interest as Trp analogs. In certain embodiments of the invention the Trp analog, e.g., at position 4, is 5-methoxy, 5-methyl-, 1-methyl-, or 1-formyl-tryptophan. In certain embodiments of the invention a Trp analog (e.g., at position 4) comprising a 1-alkyl substituent, e.g., a lower alkyl (e.g., C₁-C₅ substituent is used. In certain embodiments, N(α) methyl tryptophan or 5-methyltryptophan is used. In some embodiments, an analog comprising a 1-alkanyol substituent, e.g., a lower alkanoyl (e.g., C₁-C₅) is used. Examples include 1-acetyl-L-tryptophan and L-β-tryptophan.

In certain embodiments the Trp analog has increased hydrophobic character relative to Trp. For example, the indole ring may be substituted by one or more alkyl (e.g., methyl) groups. In certain embodiments the Trp analog participates in a hydrophobic interaction with C3. Such a Trp analog may be located, e.g., at position 4 relative to the sequence of compstatin. In certain embodiments the Trp analog comprises a substituted or unsubstituted bicyclic aromatic ring component or two or more substituted or unsubstituted monocyclic aromatic ring components.

In certain embodiments the Trp analog has increased propensity to form hydrogen bonds with C3 relative to Trp but does not have increased hydrophobic character relative to Trp. The Trp analog may have increased polarity relative to Trp and/or an increased ability to participate in an electrostatic interaction with a hydrogen bond donor on C3. Certain exemplary Trp analogs with an increased hydrogen bond forming character comprise an electronegative substituent on the indole ring. Such a Trp analog may be located, e.g., at position 7 relative to the sequence of compstatin.

In certain embodiments of the invention the compstatin analog comprises one or more Ala analogs (e.g., at position 9 relative to the sequence of compstatin), e.g., Ala analogs that are identical to Ala except that they include one or more CH₂ groups in the side chain. In certain embodiments the Ala analog is an unbranched single methyl amino acid such as 2-Abu. In certain embodiments of the invention the compstatin analog comprises one or more Trp analogs (e.g., at position 4 and/or 7 relative to the sequence of compstatin) and an Ala analog (e.g., at position 9 relative to the sequence of compstatin).

In certain embodiments of the invention the compstatin analog is a compound that comprises a peptide that has a sequence of (X′aa)ₙ-Gln-Asp-Xaa-Gly-(X″aa)ₘ, (SEQ ID NO: 2) wherein each X′aa and each X″aa is an independently selected amino acid or amino acid analog, wherein Xaa is Trp or an analog of Trp, and wherein n>1 and m>1 and n+m is between 5 and 21. The peptide has a core sequence of Gln-Asp-Xaa-Gly, where Xaa is Trp or an analog of Trp, e.g., an analog of Trp having increased propensity to form hydrogen bonds with an H-bond donor relative to Tip but, in certain embodiments, not having increased hydrophobic character relative to Trp. For example, the analog may be one in which the indole ring of Trp is substituted with an electronegative moiety, e.g., a halogen such as fluorine. In one embodiment Xaa is 5-fluorotryptophan. Absent evidence to the contrary, one of skill in the art would recognize that any non-naturally occurring peptide whose sequence comprises this core sequence and that inhibits complement activation and/or binds to C3 will have been designed based on the sequence of compstatin. In an alternative embodiment Xaa is an amino acid or amino acid analog other than a Trp analog that allows the Gln-Asp-Xaa-Gly peptide to form a β-turn.

In certain embodiments of the invention the peptide has a core sequence of X′aa-Gln-Asp-Xaa-Gly (SEQ ID NO: 3), where X′aa and Xaa are selected from Trp and analogs of Trp. In certain embodiments of the invention the peptide has a core sequence of X′aa-Gln-Asp-Xaa-Gly (SEQ ID NO: 3), where X′aa and Xaa are selected from Trp, analogs of Trp, and other amino acids or amino acid analogs comprising at least one aromatic ring. In certain embodiments of the invention the core sequence forms a β-turn in the context of the peptide. The β-turn may be flexible, allowing the peptide to assume two or more conformations as assessed for example, using nuclear magnetic resonance (NMR). In certain embodiments X′aa is an analog of Tip that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention X′aa is an analog of Tip that has increased hydrophobic character relative to Tip. For example, X′aa may be 1-methyltryptophan. In certain embodiments of the invention Xaa is an analog of Trp that has increased propensity to form hydrogen bonds relative to Tip but, in certain embodiments, not having increased hydrophobic character relative to Tip. In certain embodiments of the invention the analog of Tip that has increased propensity to form hydrogen bonds relative to Tip comprises a modification on the indole ring of Trp, e.g., at position 5, such as a substitution of a halogen atom for an H atom at position 5. For example, Xaa may be 5-fluorotryptophan.

In certain embodiments of the invention the peptide has a core sequence of X′aa-Gln-Asp-Xaa-Gly-X″aa (SEQ ID NO: 4), where X′aa and Xaa are each independently selected from Tip and analogs of Trp and X″aa is selected from His, Ala, analogs of Ala, Phe, and Tip. In certain embodiments of the invention X′aa is an analog of Tip that has increased hydrophobic character relative to Trp, such as 1-methyltryptophan or another Trp analog having an alkyl substituent on the indole ring (e.g., at position 1, 4, 5, or 6). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention Xaa is an analog of Trp that has increased propensity to form hydrogen bonds with C3 relative to Trp but, in certain embodiments, not having increased hydrophobic character relative to Tip. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan. In certain embodiments X″aa is Ala or an analog of Ala such as Abu or another unbranched single methyl amino acid. In certain embodiments of the invention the peptide has a core sequence of X′aa-Gln-Asp-Xaa-Gly-X″aa (SEQ ID NO: 4), where X′aa and Xaa are each independently selected from Trp, analogs of Trp, and amino acids or amino acid analogs comprising at least one aromatic side chain, and X″aa is selected from His, Ala, analogs of Ala, Phe, and Tip. In certain embodiments X″aa is selected from analogs of Trp, aromatic amino acids, and aromatic amino acid analogs.

In certain preferred embodiments of the invention the peptide is cyclic. The peptide may be cyclized via a bond between any two amino acids, one of which is (X′aa)ₙ and the other of which is located within (X″aa)ₘ. In certain embodiments the cyclic portion of the peptide is between 9 and 15 amino acids in length, e.g., 10-12 amino acids in length. In certain embodiments the cyclic portion of the peptide is 11 amino acids in length, with a bond (e.g., a disulfide bond) between amino acids at positions 2 and 12. For example, the peptide may be 13 amino acids long, with a bond between amino acids at positions 2 and 12 resulting in a cyclic portion 11 amino acids in length.

In certain embodiments the peptide comprises or consists of the sequence X′aa1-X′aa2-X′aa3-X′aa4-Gln-Asp-Xaa-Gly-X″aa1-X″aa2-X″aa3-X″aa4-X″aa5 (SEQ ID NO: 5). In certain embodiments X′aa4 and Xaa are selected from Tip and analogs of Trp, and X′aa1, X′aa2, X′aa3, X″aa1, X″aa2, X″aa3, X″aa4, and X″aa5 are independently selected from among amino acids and amino acid analogs. In certain embodiments X′ aa4 and Xaa are selected from aromatic amino acids and aromatic amino acid analogs. Any one or more of X′aa1, X′aa2, X′aa3, X″aa1, X″aa2, X″aa3, X″aa4, and X″aa5 may be identical to the amino acid at the corresponding position in compstatin. In one embodiment, X″aa1 is Ala or a single methyl unbranched amino acid. The peptide may be cyclized via a covalent bond between (i) X′aa1, X′aa2, or X′aa3; and (ii) X″aa2, X″aa3, X″aa4 or X″aa5. In one embodiment the peptide is cyclized via a covalent bond between X′aa2 and X″aa4. In one embodiment the covalently bound amino acid are each Cys and the covalent bond is a disulfide (S-S) bond. In other embodiments the covalent bond is a C—C, C—O, C—S, or C—N bond. In certain embodiments one of the covalently bound residues is an amino acid or amino acid analog having a side chain that comprises a primary or secondary amine, the other covalently bound residue is an amino acid or amino acid analog having a side chain that comprises a carboxylic acid group, and the covalent bond is an amide bond Amino acids or amino acid analogs having a side chain that comprises a primary or secondary amine include lysine and diaminocarboxylic acids of general structure NH₂(CH₂)—CH(NH₂)COOH such as 2,3-diaminopropionic acid (dapa), 2,4-diaminobutyric acid (daba), and ornithine (orn), wherein n=1 (dapa), 2 (daba), and 3 (orn), respectively. Examples of amino acids having a side chain that comprises a carboxylic acid group include dicarboxylic amino acids such as glutamic acid and aspartic acid. Analogs such as beta-hydroxy-L-glutamic acid may also be used. In some embodiments a peptide is cyclized with a thioether bond, e.g., as described in PCT/US2011/052442 (WO/2012/040259). For example, in some embodiments a disulfide bond in any of the peptides is replaced with a thioether bond. In some embodiments, a cystathionine is formed. In some embodiments the cystathionine is a delta-cystathionine or a gamma-cystathionine. In some embodiments a modification comprises replacement of a Cys-Cys disulfide bond between cysteines at X′aa2 and X″aa4 in SEQ ID NO: 5 (or corresponding positions in other sequences) with addition of a CH₂, to form a homocysteine at X′aa2 or X″aa4, and introduction of a thioether bond, to form a cystathionine. In one embodiment, the cystathionine is a gamma-cystathionine. In another embodiment, the cystathionine is a delta-cystathionine. Another modification of use in certain embodiments comprises replacement of the disulfide bond with a thioether bond without the addition of a CH₂, thereby forming a lantithionine. In some embodiments a compstatin analog having a thioether in place of a disulfide bond has increased stability, at least under some conditions, as compared with the compstatin analog having the disulfide bond.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 6); wherein:

Xaa1 is Ile, Val, Leu, B′-Ile, B¹-Val, B¹-Leu or a dipeptide comprising Gly-Ile or B¹-Gly-Ile, and B¹ represents a first blocking moiety;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by a second blocking moiety B²; and the two Cys residues are joined by a disulfide bond. In some embodiments, Xaa4 is Leu, Nle, His, or Phe or a depeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B²; and the two Cys residues are joined by a disulfide bond.

In other embodiments Xaa1 is absent or is any amino acid or amino acid analog, and Xaa2, Xaa2*, Xaa3, and Xaa4 are as defined above. If Xaa1 is absent, the N-terminal Cys residue may have a blocking moiety B¹ attached thereto.

In another embodiment, Xaa4 is any amino acid or amino acid analog and Xaa1, Xaa2, Xaa2*, and Xaa3 are as defined above. In another embodiment Xaa4 is a dipeptide selected from the group consisting of: Thr-Ala and Thr-Asn, wherein the carboxy terminal —OH or the Ala or Asn is optionally replaced by a second blocking moiety B².

In any of the embodiments of the compstatin analog of SEQ ID NO: 6, Xaa2 may be Trp.

In any of the embodiments of the compstatin analog of SEQ ID NO: 6, Xaa2 may be an analog of Trp comprising a substituted or unsubstituted bicyclic aromatic ring component or two or more substituted or unsubstituted monocyclic aromatic ring components. For example, the analog of Trp may be selected from 2-naphthylalanine (2-NaI), 1-naphthylalanine (1-NaI), 2-indanylglycine carboxylic acid (Ig1), dihydrotrpytophan (Dht), and 4-benzoyl-L-phenylalanine.

In any of the embodiments of the compstatin analog of SEQ ID NO: 6, Xaa2 may be an analog of Trp having increased hydrophobic character relative to Tip. For example, the analog of Trp may be selected from 1-methyltryptophan, 4-methyltryptophan, 5-methyltryptophan, and 6-methyltryptophan. In one embodiment, the analog of Trp is 1-methyltryptophan. In one embodiment, Xaa2 is 1-methyltryptophan, Xaa2* is Trp, Xaa3 is Ala, and the other amino acids are identical to those of compstatin.

In any of the embodiments of the compstatin analog of SEQ ID NO: 6, Xaa2* may be an analog of Trp such as an analog of Trp having increased hydrogen bond forming propensity with C3 relative to Trp, which, in certain embodiments, does not have increased hydrophobic character relative to Trp. In certain embodiments the analog of Trp comprises an electronegative substituent on the indole ring. For example, the analog of Trp may be selected from 5-fluorotryptophan and 6-fluorotryptophan.

In certain embodiments of the invention Xaa2 is Trp and Xaa2* is an analog of Trp having increased hydrogen bond forming propensity with C3 relative to Tip which, in certain embodiments, does not have increased hydrophobic character relative to Trp. In certain embodiments of the compstatin analog of SEQ ID NO: 6, Xaa2 is analog of Trp having increased hydrophobic character relative to Tip such as an analog of Trp selected from 1-methyltryptophan, 4-methyltryptophan, 5-methyltryptophan, and 6-methyltryptophan, and Xaa2* is an analog of Trp having increased hydrogen bond forming propensity with C3 relative to Trp which, in certain embodiments, does not have increased hydrophobic character relative to Trp. For example, in one embodiment Xaa2 is methyltryptophan and Xaa2* is 5-fluorotryptophan.

In certain of the afore-mentioned embodiments, Xaa3 is Ala. In certain of the afore-mentioned embodiments Xaa3 is a single methyl unbranched amino acid, e.g., Abu.

The invention further provides compstatin analogs of SEQ ID NO: 6, as described above, wherein Xaa2 and Xaa2* are independently selected from Trp, analogs of Trp, and other amino acids or amino acid analogs that comprise at least one aromatic ring, and Xaa3 is His, Ala or an analog of Ala, Phe, Trp, an analog of Trp, or another aromatic amino acid or aromatic amino acid analog.

In certain embodiments of the invention the blocking moiety present at the N- or C-terminus of any of the compstatin analogs described herein is any moiety that stabilizes a peptide against degradation that would otherwise occur in mammalian (e.g., human or non-human primate) blood or interstitial fluid. For example, blocking moiety B¹ could be any moiety that alters the structure of the N-terminus of a peptide so as to inhibit cleavage of a peptide bond between the N-terminal amino acid of the peptide and the adjacent amino acid. Blocking moiety B² could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention blocking moiety B¹ comprises an acyl group (i.e., the portion of a carboxylic acid that remains following removal of the —OH group). The acyl group typically comprises between 1 and 12 carbons, e.g., between 1 and 6 carbons. For example, in certain embodiments of the invention blocking moiety B¹ is selected from the group consisting of: formyl, acetyl, proprionyl, butyryl, isobutyryl, valeryl, isovaleryl, etc. In one embodiment, the blocking moiety B′ is an acetyl group, i.e., Xaa1 is Ac-Ile, Ac-Val, Ac-Leu, or Ac-Gly-Ile.

In certain embodiments of the invention blocking moiety B² is a primary or secondary amine (—NH₂ or —NHR¹, wherein R is an organic moiety such as an alkyl group).

In certain embodiments of the invention blocking moiety B¹ is any moiety that neutralizes or reduces the positive charge that may otherwise be present at the N-terminus at physiological pH. In certain embodiments of the invention blocking moiety B² is any moiety that neutralizes or reduces the negative charge that may otherwise be present at the C-terminus at physiological pH.

In certain embodiments of the invention, the compstatin analog is acetylated or amidated at the N-terminus and/or C-terminus, respectively. A compstatin analog may be acetylated at the N-terminus, amidated at the C-terminus, and or both acetylated at the N-terminus and amidated at the C-terminus. In certain embodiments of the invention a compstatin analog comprises an alkyl or aryl group at the N-terminus rather than an acetyl group.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 7); wherein:

Xaa1 is Ile, Val, Leu, Ac-Ile, Ac-Val, Ac-Leu or a dipeptide comprising Gly-Ile or Ac-Gly-Ile;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of L-Thr, D-Thr, Ile, Val, Gly, Ala, or Asn optionally is replaced by —NH₂; and the two Cys residues are joined by a disulfide bond. In some embodiments, Xaa4 is Leu, Nle, His, or Phe or a depeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B2; and the two Cys residues are joined by a disulfide bond.

In some embodiments, Xaa1, Xaa2, Xaa2*, Xaa3, and Xaa4 are as described above for the various embodiments of SEQ ID NO: 6. For example, in certain embodiments Xaa2* is Trp. In certain embodiments Xaa2 is an analog of Trp having increased hydrophobic character relative to Trp, e.g., 1-methyltryptophan. In certain embodiments Xaa3 is Ala. In certain embodiments Xaa3 is a single methyl unbranched amino acid.

In certain embodiments of the invention Xaa1 is Ile and Xaa4 is L-Thr.

In certain embodiments of the invention Xaa1 is Ile, Xaa2* is Trp, and Xaa4 is L-Thr.

The invention further provides compstatin analogs of SEQ ID NO: 7, as described above, wherein Xaa2 and Xaa2* are independently selected from Trp, analogs of Trp, other amino acids or aromatic amino acid analogs, and Xaa3 is His, Ala or an analog of Ala, Phe, Trp, an analog of Trp, or another aromatic amino acid or aromatic amino acid analog.

In certain embodiments of any of the compstatin analogs described herein, an analog of Phe is used rather than Phe.

Table 2 provides a non-limiting list of compstatin analogs useful in the present invention. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 2 are amidated at the C-terminus. Bold text is used to indicate certain modifications. Activity relative to compstatin is based on published data and assays described therein (WO2004/026328, WO2007044668, Mallik, 2005; Katragadda, 2006). Where multiple publications reporting an activity were consulted, the more recently published value is used, and it will be recognized that values may be adjusted in the case of differences between assays. It will also be appreciated that in certain embodiments of the invention the peptides listed in Table 2 are cyclized via a disulfide bond between the two Cys residues when used in the therapeutic compositions and methods of the invention. Alternate means for cyclizing the peptides are also within the scope of the invention. As noted above, in various embodiments of the invention one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments of the invention, for example, a compstatin analog comprises an N-methyl glycine, e.g., at the position corresponding to position 8 of compstatin and/or at the position corresponding to position 13 of compstatin. In some embodiments, one or more of the compstatin analogs in Table 2 contains at least one N-methyl glycine, e.g., at the position corresponding to position 8 of compstatin and/or at the position corresponding to position 13 of compstatin. In some embodiments, one or more of the compstatin analogs in contains at least one N-methyl isoleucine, e.g., at the position corresponding to position 13 of compstatin. For example, a Thr at or near the C-terminal end of a peptide whose sequence is listed in Table 2 may be replaced by N-methyl Ile. As will be appreciated, in some embodiments the N-methylated amino acids comprise N-methyl Gly at position 8 and N-methyl Ile at position 13. In some embodiments the N-methylated amino acids comprise N-methyl Gly in a core sequence such as SEQ ID NO: 3 or SEQ ID NO: 4.

TABLE 2

| | | | Activ- |
|---|---|---|---|
| | | | ity |
| | | SEQ | over |
| | | ID | comp |
| Peptide | Sequence | NO: | statin |
| Compstatin | H-ICVVQDWGHHRCT- | 8 | * |
| | CONH2 | | |
| Ac-compstatin | Ac-ICVVQDWGHHRCT- | 9 | 3x |
| | CONH2 | | more |
| Ac-V4Y/H9A | Ac-ICVYQDWGAHRCT- | 10 | 14x |
| | CONH2 | | more |
| Ac-V4W/H9A-OH | Ac-ICVWQDWGAHRCT- | 11 | 27x |
| | COOH | | more |
| Ac-V4W/H9A | Ac-ICVWQDWGAHRCT- | 12 | 45x |
| | CONH2 | | more |
| Ac-V4W/H9A/ | Ac-ICVWQDWGAHRCdT- | 13 | 55x |
| T13dT-OH | COOH | | more |
| Ac-V4(2-Nal)/ | Ac-ICV(2-Nal)QDWGAHRCT- | 14 | 99x |
| H9A | CONH2 | | more |
| Ac V4(2-Nal)/ | Ac-ICV(2-Nal)QDWGAHRT- | 15 | 38x |
| H9A-OH | COOH | | more |
| Ac V4(1-Nal)/ | Ac-ICV(1-Nal)QDWGAHRCT- | 16 | 30x |
| H9A-OH | COOH | | more |
| Ac-V42lgl/ | Ac-ICV(2-lgl)QDWGAHRCT- | 17 | 39x |
| H9A | CONH2 | | more |
| Ac-V42lgl/ | Ac-ICV(2-lgl)QDWGAHRCT- | 18 | 37x |
| H9A-OH | COOH | | more |
| Ac-V4Dht/ | Ac-ICVDhtQDWGAHRCT- | 19 | 5x |
| H9A-OH | COOH | | more |
| Ac-V4(Bpa)/ | Ac-ICV(Bpa)QDWGAHRCT- | 20 | 49x |
| H9A-OH | COOH | | more |
| Ac-V4(Bpa)/ | Ac-ICV(Bpa)QDWGAHRCT- | 21 | 86x |
| H9A | CONH2 | | more |
| Ac-V4(Bta)/ | Ac-ICV(Bta)QDWGAHRCT- | 22 | 65x |
| H9A-OH | COOH | | more |
| Ac-V4(Bta)/ | Ac-ICV(Bta)QDWGAHRCT- | 23 | 64x |
| H9A | CONH2 | | more |
| Ac-V4W/H9 | Ac-ICVWQDWG(2-Abu)HRCT- | 24 | 64x |
| (2-Abu) | CONH2 | | more |
| +G/V4W/H9A + | H-GICVWQDWGAHRCTAN- | 25 | 38x |
| AN-OH | COOH | | more |
| Ac-V4(5fW)/ | Ac-ICV(5fW)QDWGAHRCT- | 26 | 31x |
| H9A | CONH₂ | | more |
| Ac-V4(5-MeW)/ | Ac-ICV(5-methyl-W) | 27 | 67x |
| H9A | QDWGAHRCT-CONH₂ | | more |
| Ac-V4(1-MeW)/ | Ac-ICV(1-methyl-W) | 28 | 264x |
| H9A | QDWGAHRCT-CONH₂ | | more |
| Ac-V4W/W7(5fW)/ | Ac-ICVWQD(5fW)GAHRCT- | 29 | 121x |
| H9A | CONH₂ | | more |
| Ac-V4(5fW)/W7 | Ac-ICV(5fW)QD(5fW)GAHRCT- | 30 | NA |
| (5fW)/H9A | CONH₂ | | |
| Ac-V4(5-MeW)/ | Ac-ICV(5-methyl-W)QD(5fW) | 31 | NA |
| W7(5fW)H9A | GAHRCT-CONH₂ | | |
| Ac-V4(1MeW)/ | Ac-ICV(1-methyl-W)QD(5fW) | 32 | 264x |
| W7(5fW)/H9A | GAHRCT-CONH₂ | | more |
| +G/V4(6fW)/W7 | H-GICV(6fW)QD(6fW) | 33 | 126x |
| (6fW)H9A + | GAHRCTN-COOH | | more |
| N-OH | | | |
| Ac-V4(1-formyl- | Ac-ICV(1-formyl-W) | 34 | 264x |
| W)/H9A | QDWGAHRCT-CONH₂ | | more |
| Ac-V4(5-methoxy- | Ac-ICV(1-methyoxy-W) | 35 | 76x |
| W)/H9A | QDWGAHRCT-CONH₂ | | more |
| G/V4(5f-W)/W7 | H-GICV(5fW)QD(5fW) | 36 | 112x |
| (5fW)/H9A + | GAHRCTN-COOH | | more |
| N-OH | | | |

NA = not available

In certain embodiments of the compositions and methods of the invention the compstatin analog has a sequence selected from sequences 9-36. In certain embodiments of the compositions and methods of the invention the compstatin analog has a sequence selected from SEQ ID NOs: 14, 21, 28, 29, 32, 33, 34, and 36. In certain embodiments of the compositions and/or methods of the invention the compstatin analog has a sequence selected from SEQ ID NOs: 30 and 31. In one embodiment of the compositions and methods of the invention the compstatin analog has a sequence of SEQ ID NO: 28. In one embodiment of the compositions and methods of the invention the compstatin analog has a sequence of SEQ ID NO: 32. In one embodiment of the compositions and methods of the invention the compstatin analog has a sequence of SEQ ID NO: 34. In one embodiment of the compositions and methods of the invention the compstatin analog has a sequence of SEQ ID NO: 36.

In some embodiments a blocking moiety B¹ comprises an amino acid, which may be represented as Xaa0. In some embodiments blocking moiety B² comprises an amino acid, which may be represented as XaaN. In some embodiments blocking moiety B¹ and/or B² comprises a non-standard amino acid, such as a D-amino acid, N-alkyl amino acid (e.g., N-methyl amino acid). In some embodiments a blocking moiety B¹ and/or B² comprises a non-standard amino acid that is an analog of a standard amino acid. In some embodiments an amino acid analog comprises a lower alkyl, lower alkoxy, or halogen substituent, as compared with a standard amino acid of which it is an analog. In some embodiments a substituent is on a side chain. In some embodiments a substituent is on an alpha carbon atom. In some embodiments, a blocking moiety B¹ comprising an amino acid, e.g., a non-standard amino acid, further comprises a moiety B^{1a}. For example, blocking moiety B¹ may be represented as B^{1a}-Xaa0. In some embodiments B^{1a} neutralizes or reduces a positive charge that may otherwise be present at the N-terminus at physiological pH. In some embodiments B^{1a} comprises or consists of, e.g., an acyl group that, e.g., comprises between 1 and 12 carbons, e.g., between 1 and 6 carbons. In certain embodiments blocking moiety B^{1a} is selected from the group consisting of: formyl, acetyl, proprionyl, butyryl, isobutyryl, valeryl, isovaleryl, etc. In some embodiments, a blocking moiety B² comprising an amino acid, e.g., a non-standard amino acid, may further comprise a moiety B^{2a} For example, blocking moiety B² may be represented as XaaN-B^{2a}, where N represents the appropriate number for the amino acid (which will depend on the numbering used in the rest of the peptide). In some embodiments B^{2a} neutralizes or reduces a negative charge that may otherwise be present at the C-terminus at physiological pH. In some embodiments B^{2a} comprises or consists of a primary or secondary amine (e.g., NH₂). It will be understood that a blocking activity of moiety B^{1a}-Xaa0 and/or XaaN-B^{2a} may be provided by either or both components of the moiety in various embodiments. In some embodiments a blocking moiety or portion thereof, e.g., an amino acid residue, may contribute to increasing affinity of the compound for C3 or C3b and/or improve the activity of the compound. In some embodiments a contribution to affinity or activity of an amino acid residue may be at least as important as a contribution to blocking activity. For example, in some embodiments Xaa0 and/or XaaN in B^{1a}-Xaa0 and/or XaaN-B²a may function mainly to increase affinity or activity of the compound, while B^{1a} and/or B^{2a} may inhibit digestion of and/or neutralize a charge of the peptide. In some embodiments a compstatin analog comprises the amino acid sequence of any of SEQ ID NOs: 5-36, wherein SEQ ID NOs: 5-36 is further extended at the N- and/or C-terminus. In some embodiments, the sequence may be represented as B^{1a}-Xaa0-SEQUENCE-XaaN-B^{2a}, where SEQUENCE represents any of SEQ ID NOs: 5-36, wherein B^{1a} and B^{2a} may independently be present or absent. For example, in some embodiments a compstatin analog comprises B^{1a}-Xaa0-X′aa1-X′aa2-X′aa3-X′aa4-Gln-Asp-Xaa-Gly-X″aa1-X″aa2-X″aa3-X″aa4-X″aa5-XaaN-B^{2a} (SEQ ID NO: 37), where X′aa1-X′aa2-X′aa3-X′aa4, Xaa, X″aa1, X″aa2, X″aa3, X″aa4, and X″aa5 are as set forth above for SEQ ID NO: 5.

In some embodiments a compstatin analog comprises B^{1a}-Xaa0-Xaa1-Cys-Val-Xaa2-Gln-Asp-Xaa2*-Gly-Xaa3-His-Arg-Cys-Xaa4-XaaN-B^{2a} (SEQ ID NO: 38), where Xaa1, Xaa2, Xaa2*, Xaa3, and Xaa4 are as set forth above for SEQ ID NO: 6 or wherein Xaa1, Xaa2, Xaa2*, Xaa3, and Xaa4 are as set forth for SEQ ID NO: 6 or SEQ ID NO: 7.

In some embodiments a compstatin analog comprises B^{1a}-Xaa0-Xaa1-Xaa2-Xaa3-Xaa4-Xaa5-Xaa6-Xaa7-Xaa8-Xaa9-Xaa10-Xaa11-Xaa12-Xaa13-XaaN-B^{2a} (SEQ ID NO: 39) wherein Xaa1, Xaa2, Xaa3, Xaa4, Xaa5, Xaa6, Xaa7, Xaa8, Xaa9, Xaa10, Xaa11, Xaa12, and Xaa13 are identical to amino acids at positions 1-13 of any of SEQ ID NOs: 9-36.

In some embodiments Xaa0 and/or XaaN in any compstatin analog sequence comprises an amino acid that comprises an aromatic ring having an alkyl substituent at one or more positions. In some embodiments an alkyl substituent is a lower alkyl substituent. For example, in some embodiments an alkyl substituent is a methyl or ethyl group. In some embodiments a substituent is located at any position that does not destroy the aromatic character of the compound. In some embodiments a substituent is located at any position that does not destroy the aromatic character of a ring to which the substituent is attached. In some embodiments a substituent is located at position 1, 2, 3, 4, or 5. In some embodiments Xaa0 comprises an O-methyl analog of tyrosine, 2-hydroxyphenylalanine or 3-hydroxyphenylalanine. For purposes of the present disclosure, a lower case “m” followed by a three letter amino acid abbreviation may be used to specifically indicate that the amino acid is an N-methyl amino acid. For example, where the abbreviation “mGly” appears herein, it denotes N-methyl glycine (also sometimes referred to as sarcosine or Sar). In some embodiments Xaa0 is or comprises mGly, Tyr, Phe, Arg, Tip, Thr, Tyr(Me), Cha, mPhe, mVal, mIle, mAla, DTyr, DPhe, DArg, DTrp, DThr, DTyr(Me), mPhe, mVal, mIle, DAla, or DCha. For example, in some embodiments a compstatin analog comprises a peptide having a sequence B¹-Ile-[Cys-Val-Trp(Me)-Gln-Asp-Trp-mGly-Ala-His-Arg-Cys]-mIle-B² (SEQ ID NO: 40) or B¹-Ile-[Cys-Val-Trp(Me)-Gln-Asp-Trp-mGly-Ala-His-Arg-Cys]-mIle-B² (SEQ ID NO: 41). The two Cys residues are joined by a disulfide bond in the active compounds. In some embodiments the peptide is acetylated at the N-terminus and/or amidated at the C-terminus. In some embodiments B¹ comprises B^{1a}-Xaa0 and/or B² comprises XaaN-B^{2a}, as described above. For example, in some embodiments B¹ comprises or consists of Gly, mGly, Tyr, Phe, Arg, Trp, Thr, Tyr(Me), mPhe, mVal, mIle, mAla, DTyr, DPhe, DTrp, DCha, DAla and B² comprises NH₂, e.g., a carboxy terminal —OH of mIle is replaced by NH₂. In some embodiments B¹ comprises or consists of mGly, Tyr, DTyr, or Tyr(Me) and B² comprises NH₂, e.g., a carboxy terminal —OH of mIle is replaced by NH₂. In some embodiments an Ile at position Xaa1 is replaced by Gly. Complement inhibition potency and/or C3b binding parameters of selected compstatin analogs are described in WO/2010/127336 (PCT/US2010/033345) and/or in Qu, et al, Immunobiology (2012), doi: 10.1016/j.imbio.2012.06.003.

In some embodiments a blocking moiety or portion thereof, e.g., an amino acid residue, may contribute to increasing affinity of the compound for C3 or C3b and/or improve the activity of the compound. In some embodiments a contribution to affinity or activity of an amino acid or amino acid analog may be more significant than a blocking activity.

In certain embodiments of the compositions and methods of the invention the compstatin analog has a sequence as set forth in Table 2, but where the Ac-group is replaced by an alternate blocking moiety B¹, as described herein. In some embodiments the —NH₂ group is replaced by an alternate blocking moiety B², as described herein.

In one embodiment, the compstatin analog binds to substantially the same region of the β chain of human C3 as does compstatin. In one embodiment the compstatin analog is a compound that binds to a fragment of the C-terminal portion of the β chain of human C3 having a molecular weight of about 40 kDa to which compstatin binds (Soulika, A. M., et al., Mol. Immunol., 35:160, 1998; Soulika, A. M., et al., Mol. Immunol. 43(12):2023-9, 2006). In certain embodiments the compstatin analog is a compound that binds to the binding site of compstatin as determined in a compstatin-C3 structure, e.g., a crystal structure or NMR-derived 3D structure. In certain embodiments the compstatin analog is a compound that could substitute for compstatin in a compstatin-C3 structure and would form substantially the same intermolecular contacts with C3 as compstatin. In certain embodiments the compstatin analog is a compound that binds to the binding site of a peptide having a sequence set forth in Table 2, e.g., SEQ ID NO: 14, 21, 28, 29, 32, 33, 34, or 36 or another compstatin analog sequence disclosed herein in a peptide-C3 structure, e.g., a crystal structure. In certain embodiments the compstatin analog is a compound that binds to the binding site of a peptide having SEQ ID NO: 30 or 31 in a peptide-C3 structure, e.g., a crystal structure. In certain embodiments the compstatin analog is a compound that could substitute for the peptide of SEQ ID NO: 9-36, e.g., a compound that could substitute for the peptide of SEQ ID NO: 14, 21, 28, 29, 32, 33, 34, or 36 or another compstatin analog sequence disclosed herein in a peptide-C3 structure and would form substantially the same intermolecular contacts with C3 as the peptide. In certain embodiments the compstatin analog is a compound that could substitute for the peptide of SEQ ID NO: 30 or 31 in a peptide-C3 structure and would form substantially the same intermolecular contacts with C3 as the peptide.

One of ordinary skill in the art will readily be able to determine whether a compstatin analog binds to a fragment of the C-terminal portion of the β chain of C3 using routine experimental methods. For example, one of skill in the art could synthesize a photocrosslinkable version of the compstatin analog by including a photo-crosslinking amino acid such as p-benzoyl-L-phenylalanine (Bpa) in the compound, e.g., at the C-terminus of the sequence (Soulika, A. M., et al, supra). Optionally additional amino acids, e.g., an epitope tag such as a FLAG tag or an HA tag could be included to facilitate detection of the compound, e.g., by Western blotting. The compstatin analog is incubated with the fragment and crosslinking is initiated. Colocalization of the compstatin analog and the C3 fragment indicates binding. Surface plasmon resonance may also be used to determine whether a compstatin analog binds to the compstatin binding site on C3 or a fragment thereof. One of skill in the art would be able to use molecular modeling software programs to predict whether a compound would form substantially the same intermolecular contacts with C3 as would compstatin or a peptide having the sequence of any of the peptides in Table 2, e.g., SEQ ID NO: 14, 21, 28, 29, 32, 33, 34, or 36, or in some embodiments SEQ ID NO: 30 or 31 or another compstatin analog sequence disclosed herein.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) or similar molecules to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deilv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, Calif., which also provides details of appropriate conjugation procedures. In another embodiment a compstatin analog is fused to the Fc domain of an immunoglobulin or a portion thereof. In some other embodiments a compstatin analog is conjugated to an albumin moiety or to an albumin binding peptide. Thus in some embodiments a compstatin analog is modified with one or more polypeptide or non-polypeptide components, e.g., the compstatin analog is pegylated or conjugated to another moiety. In some embodiments the component is not the Fe domain of an immunoglobulin or a portion thereof. A compstatin analog can be provided as a multimer or as part of a supramolecular complex, which can include either a single molecular species or multiple different species (e.g., multiple different analogs).

In some embodiments, a compstatin analog is a multivalent compound comprising a plurality of compstatin analog moieties covalently or noncovalently linked to a polymeric backbone or scaffold. The compstatin analog moieties can be identical or different. In certain embodiments of the invention the multivalent compound comprises multiple instances, or copies, of a single compstatin analog moiety. In other embodiments of the invention the multivalent compound comprises one or more instances of each of two of more non-identical compstatin analog moieties, e.g., 3, 4, 5, or more different compstatin analog moieties. In certain embodiments of the invention the number of compstatin analog moieties (“n”) is between 2 and 6. In other embodiments of the invention n is between 7 and 20. In other embodiments of the invention n is between 20 and 100. In other embodiments n is between 100 and 1,000. In other embodiments of the invention n is between 1,000 and 10,000. In other embodiments n is between 10,000 and 50,000. In other embodiments n is between 50,000 and 100,000. In other embodiments n is between 100,000 and 1,000,000.

The compstatin analog moieties may be attached directly to the polymeric scaffold or may be attached via a linking moiety that connects the compstatin analog moiety to the polymeric scaffold. The linking moiety may be attached to a single compstatin analog moiety and to the polymeric scaffold. Alternately, a linking moiety may have multiple compstatin analog moieties joined thereto so that the linking moiety attaches multiple compstatin analog moieties to the polymeric scaffold.

In some embodiments, a compstatin analog comprises an amino acid having a side chain comprising a primary or secondary amine, e.g., a Lys residue. For example, any of the compstatin analog sequences disclosed herein may be extended or modified by addition of a linker comprising one or more amino acids, e.g., one or more amino acids comprising a primary or secondary amine, e.g., in a side chain thereof. For example, a Lys residue, or a sequence comprising a Lys residue, is added at the N-terminus and/or C-terminus of the compstatin analog. In some embodiments, the Lys residue is separated from the cyclic portion of the compstatin analog by a rigid or flexible spacer. A linker or spacer may, for example, comprise a substituted or unsubstituted, saturated or unsaturated alkyl chain, oligo(ethylene glycol) chain, and/or other moieties. The length of the chain may be, e.g., between 2 and 20 carbon atoms. In some embodiments the spacer is or comprises a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. A PEG moiety or similar molecule or polymeric scaffold may be linked to the primary or secondary amine, optionally via a linker. In some embodiments, a bifunctional linker is used. Abifunctional linker may comprise two reactive functional groups, which may be the same or different in various embodiments. In various embodiments, one or more linkers, spacers, and/or techniques of conjugation described in Hermanson, supra, is used.

Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In another embodiment, short chains are prepolymerized, functionalized, and then a mixture of short chains of different composition are assembled into longer polymers.

In some aspects a moiety such as a polyethylene glycol (PEG) chain, polyoxazoline (POZ) chain, or other polymer(s), e.g., polypeptides, that, e.g., stabilize the compound, increase its lifetime in the body, increase its solubility, decrease its immunogenicity, and/or increase its resistance to degradation may be referred to herein as a “clearance reducing moiety” (CRM), and a compstatin analog comprising such a moiety may be referred to as a long-acting compstatin analog. In some embodiments a CRM comprises a non-polypeptide polymer or polypeptide that has a molecular weight of between 5 kD and 150 kD, e.g., at least 5, 10, 20, 30, 40, 50, 60, 70, 80, 90, 100 kd, or more, e.g., between 100 and 120, or 120 and 150 kD. In some embodiments the polymer is linear. In some embodiments the polymer is branched.

In some aspects, a long-acting compstatin analog comprises a compound of formula M-L-A, wherein A is a moiety that comprises a CRM, L is an optionally present linking portion, and M comprises a compstatin analog moiety. The compstatin analog moiety can comprise any compstatin analog, e.g., any compstatin analog described above, in various embodiments. Formula M-L-A encompasses embodiments in which L-A is present at the N-terminus of the compstatin analog moiety, embodiments in which L-A is present at the C-terminus of the compstatin analog moiety, embodiments in which L-A is attached to a side chain of an amino acid of the compstatin analog moiety, and embodiments where the same or different L-As are present at both ends of M. In some embodiments the same or different M (or M-L) may be present at the ends of a linear CRM or at the termini of branches of a branched CRM. It will be appreciated that when certain compstatin analog(s) are present as a compstatin analog moiety in a compound of formula M-L-A, a functional group of the compstatin analog will have reacted with a functional group of L to form a covalent bond to A or L. For example, a long-acting compstatin analog in which the compstatin analog moiety comprises a compstatin analog that contains an amino acid with a side chain containing a primary amine (NH₂) group (which compstatin analog can be represented by formula R¹—(NH₂)), can have a formula R¹—NH-L-A in which a new covalent bond to L (e.g., N—C) has been formed and a hydrogen lost. Thus the term “compstatin analog moiety” includes molecular structures in which at least one atom of a compstatin analog participates in a covalent bond with a second moiety, which may, e.g., modification of a side chain. Similar considerations apply to compstatin analog moieties present in multivalent compounds. In some embodiments, a blocking moiety at the N-terminus or C-terminus of a compstatin analog is replaced by L-A in the structure of a long-acting compstatin analog.

In some embodiments, L comprises an unsaturated moiety such as —CH═CH— or —CH₂—CH═CH—; a moiety comprising a non-aromatic cyclic ring system (e.g., a cyclohexyl moiety), an aromatic moiety (e.g., an aromatic cyclic ring system such as a phenyl moiety); an ether moiety (—C—O—C—); an amide moiety (—C(═O)—N—); an ester moiety (—CO—O—); a carbonyl moiety (—C(═O)—); an imine moiety (—C═N—); a thioether moiety (—C—S—C—); an amino acid residue; and/or any moiety that can be formed by the reaction of two compatible reactive functional groups. In certain embodiments, one or more moieties of a linking portion is/are substituted by independent replacement of one or more of the hydrogen (or other) atoms thereon with one or more moieties including, but not limited to aliphatic; aromatic, aryl; alkyl, aralkyl, alkanoyl, aroyl, alkoxy; thio; F; Cl; Br; I; —NO2; —CN; —CF3; —CH2CF3; —CHC12; —CH2OH; —CH2CH2OH; —CH2NH2; —CH2SO2CH3; - or -GRG1 wherein G is —O—, —S—, —NRG2-, —C(═O)—, —S(═O)—, —SO2-, —C(═O)O—, —C(═O)NRG2-, —OC(═O)—, —NRG2C(═O)—, —OC(═O)O—, —OC(═O)NRG2-, —NRG2C(═O)O—, —NRG2C(═O)NRG2-, —C(═S)—, —C(═S)S—, —SC(═S)—, —SC(═S)S—, —C(═NRG2)-, —C(═NRG2)O—, —C(═NRG2)NRG3-, —OC(═NRG2)-, —NRG2C(═NRG3)-, —NRG2SO2-, —NRG2SO2NRG3-, or —SO2NRG2-, wherein each occurrence of RG1, RG2 and RG3 independently includes, but is not limited to, hydrogen, halogen, or an optionally substituted aliphatic, aromatic, or aryl moiety. It will be appreciated that cyclic ring systems when present as substituents may optionally be attached via a linear moiety. Combinations of substituents and variables are preferably those that result in the formation of stable compounds useful in any one or more of the methods described herein.

In some embodiments, a compstatin analog of use in compositions and methods described herein is a long-acting compstatin analog that has a terminal half-life of at least 3, 4, 5, 6, or 7 days. In some embodiments such a long-acting compstatin analog is a pegylated compstatin analog or a compstatin analog comprising a recombinant polypeptide. In some embodiments a long-acting compstatin analog comprises any of SEQ ID NOs: 3-41.

Exemplary compstatin analogs, e.g., long-acting compstatin analogs, are described in any of the following: PCT Application No. PCT/US2012/054180 (published as WO/2013/036778); U.S. Ser. No. 14/343,003 (published as US Pat. Pub. No. 20150158915) PCT/US2012/037648 (published as WO/2012/155107); U.S. Ser. No. 14/116,591 (published as US Pat. Pub. No. 20140323407); PCT/US2013/070424 (published as WO/2014/078734); PCT/US2013/070417 (published as WO/2014/078731), U.S. Ser. No. 14/443,143 U.S. Ser. No. 14/443,143.

In some embodiments, a long-acting compstatin analog has an average plasma half-life of at least 1 day, e.g., 1-3 days, 3-7 days, 7-14 days, or 14-28 days, when administered IV at a dose of 10 mg/kg to humans or to non-human primates. In some embodiments, average plasma half-life of a long-acting compstatin analog following administration IV at a dose of 10 mg/kg to humans or to non-human primates is increased by at least a factor of 2, e.g., by a factor of 2-5, 5-10, 10-50, or 50-100-fold as compared with that of a corresponding compstatin analog having the same amino acid sequence (and, if applicable, one or more blocking moiet(ies)) but not comprising the CRM. In some embodiments, a plasma half-life is a terminal half-life after administration of a single IV dose. In some embodiments, a plasma half-life is a terminal half-life after steady state has been reached following administration of multiple IV doses.

The structure of compstatin is known in the art, and NMR structures for a number of compstatin analogs having higher activity than compstatin are also known (Malik, supra). Structural information may be used to design compstatin mimetics. In some embodiments, a compstatin mimetic is any compound that competes with compstatin or any compstatin analog (e.g., a compstatin analog whose sequence is set forth in Table 2) for binding to C3 or a fragment thereof (such as a 40 kD fragment of the β chain to which compstatin binds). In some embodiments, the compstatin mimetic has an activity equal to or greater than that of compstatin. In some embodiments, the compstatin mimetic is more stable, orally available, or has a better bioavailability than compstatin. The compstatin mimetic may be a peptide, nucleic acid, or small molecule. In certain embodiments the compstatin mimetic is a compound that binds to the binding site of compstatin as determined in a compstatin-C3 structure, e.g., a crystal structure or a 3-D structure derived from NMR experiments. In certain embodiments the compstatin mimetic is a compound that could substitute for compstatin in a compstatin-C3 structure and would form substantially the same intermolecular contacts with C3 as compstatin. In certain embodiments the compstatin mimetic is a compound that binds to the binding site of a peptide having a sequence set forth in Table 2, e.g., SEQ ID NO: 14, 21, 28, 29, 32, 33, 34, or 36 or other compstatin analog sequence or in certain embodiments SEQ ID NO: 30 or 31, in a peptide-C3 structure. In certain embodiments the compstatin mimetic is a compound that could substitute for a peptide having a sequence set forth in Table 2, e.g., SEQ ID NO: 14, 21, 28, 29, 32, 33, 34, or 36 or other compstatin analog sequence or in certain embodiments SEQ ID NO: 30 or 31, in a peptide-C3 structure and would form substantially the same intermolecular contacts with C3 as the peptide. In certain embodiments the compstatin mimetic has a non-peptide backbone but has side chains arranged in a sequence designed based on the sequence of compstatin.

One of skill in the art will appreciate that once a particular desired conformation of a short peptide has been ascertained, methods for designing a peptide or peptidomimetic to fit that conformation are well known. See, e.g., G. R. Marshall (1993), Tetrahedron, 49: 3547-3558; Hruby and Nikiforovich (1991), in Molecular Conformation and Biological Interactions, P. Balaram & S. Ramasehan, eds., Indian Acad. of Sci., Bangalore, PP. 429-455), Eguchi M, Kahn M., Mini Rev Med Chem., 2(5):447-62, 2002. Of particular relevance to the present invention, the design of peptide analogs may be further refined by considering the contribution of various side chains of amino acid residues, e.g., for the effect of functional groups or for steric considerations as described in the art for compstatin and analogs thereof, among others.

It will be appreciated by those of skill in the art that a peptide mimic may serve equally well as a peptide for the purpose of providing the specific backbone conformation and side chain functionalities required for binding to C3 and inhibiting complement activation. Accordingly, it is contemplated as being within the scope of the present invention to produce and utilize C3-binding, complement-inhibiting compounds through the use of either naturally-occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “peptidomimetic” or “isosteric mimetic,” to designate substitutions or derivations of a peptide that possesses much the same backbone conformational features and/or other functionalities, so as to be sufficiently similar to the exemplified peptides to inhibit complement activation. More generally, a compstatin mimetic is any compound that would position pharmacophores similarly to their positioning in compstatin, even if the backbone differs.

The use of peptidomimetics for the development of high-affinity peptide analogs is well known in the art. Assuming rotational constraints similar to those of amino acid residues within a peptide, analogs comprising non-amino acid moieties may be analyzed, and their conformational motifs verified, by means of the Ramachandran plot (Hruby & Nikiforovich 1991), among other known techniques.

One of skill in the art will readily be able to establish suitable screening assays to identify additional compstatin mimetics and to select those having desired inhibitory activities. For example, compstatin or an analog thereof could be labeled (e.g., with a radioactive or fluorescent label) and contacted with C3 in the presence of different concentrations of a test compound. The ability of the test compound to diminish binding of the compstatin analog to C3 is evaluated. A test compound that significantly diminishes binding of the compstatin analog to C3 is a candidate compstatin mimetic. For example, a test compound that diminishes steady-state concentration of a compstatin analog-C3 complex, or that diminishes the rate of formation of a compstatin analog-C3 complex by at least 25%, or by at least 50%, is a candidate compstatin mimetic. One of skill in the art will recognize that a number of variations of this screening assay may be employed. Compounds to be screened include natural products, libraries of aptamers, phage display libraries, compound libraries synthesized using combinatorial chemistry, etc. The invention encompasses synthesizing a combinatorial library of compounds based upon the core sequence described above and screening the library to identify compstatin mimetics. Any of these methods could also be used to identify new compstatin analogs having higher inhibitory activity than compstatin analogs tested thus far.

Other Compounds that Inhibit C3 Activation or Activity

Other compounds, e.g., polypeptides, small molecules, antibodies (e.g., monoclonal antibodies), aptamers, etc., that bind to C3 or C3a receptors (C3aR) are of use in certain embodiments of the invention. In certain embodiments the complement inhibitor comprises an Efb protein from Staphylococcus aureus or a variant or derivative or mimetic thereof that can bind to C3 and inhibit its activation and/or bind to and inhibit C3b. Exemplary agents are described in PCT Application Pub. WO/2004/094600. In certain embodiments the complement inhibitor comprises a Staphylococcus complement inhibitor (SCIN) protein from Staphylococcus aureus or a variant or derivative or mimetic of such protein that can bind to C3 convertase and inhibit its activation and/or bind to and inhibit C3b. Aptamers that bind to and inhibit C3 may be identified using methods such as SELEX. U.S. Pat. Pub. No. 20030191084 discloses aptamers that bind to C1q, C3 and C5. Exemplary antibodies that bind to C3 are disclosed in U.S. Pat. Pub. No. 20100111946 and/or 20120288493.

In some embodiments, a protease that degrades C3 may be used as a complement inhibitor. For example, U.S. Pat. No. 6,676,943 discloses human complement C3-degrading protein from Streptococcus pneumoniae. Such proteins, or variants thereof, may be used in certain embodiments of the invention.

U.S. Pat. No. 5,942,405, PCT/IB2006/002557 (WO/2007/034277-ARYL SUBSTITUTED IMIDAZO [4,5-C] PYRIDINE COMPOUNDS AS C3A RECEPTOR ANTAGONISTS); PCT/IB2006/002568 (WO/2007/034282-DIARYL-IMIDAZOLE COMPOUNDS CONDENSED WITH A HETEROCYCLE AS C3A RECEPTOR ANTAGONISTS) PCT/IB2006/002561 (WO2007034278-FUSED IMIDAZOLE DERIVATIVES AS C3A RECEPTOR ANTAGONISTS) PCT/US2007/026237 (WO2008079371) MODULATORS OF C3A RECEPTOR AND METHODS OF USE THEREOF disclose exemplary C3aR antagonists. In some embodiments, an RNAi agent that inhibits expression of C3 or C3aR may be used.

Compounds that Inhibit Factor B Activation or Activity

In certain embodiments a complement inhibitor inhibits activation or activity of factor B. For example, the complement inhibitor may bind to factor B and, e.g., inhibit activation of factor B. Exemplary agents that inhibit activation or activity of factor B include, e.g., antibodies (e.g., monoclonal antibodies), antibody fragments, peptides, small molecules, and aptamers. Exemplary antibodies that inhibit factor B are described in U.S. Pat. Pub. No. 20050260198. In certain embodiments an antibody or antigen-binding fragment selectively binds to factor B within the third short consensus repeat (SCR) domain. In certain embodiments the antibody prevents formation of a C3bBb complex. In certain embodiments the antibody or antigen-binding fragment prevents or inhibits cleavage of factor B by factor D. In some embodiments, an antibody binds to the Bb portion of factor B. PCT/US2008/074489 (WO/2009/029669) discloses exemplary antibodies, e.g., the antibody produced by the hybridoma clone deposited under ATCC Accession Number PTA-8543. In some embodiments, a humanized version of said antibody is used, which may be an antibody fragment. In certain embodiments a complement inhibitor, e.g., antibody, small molecule, aptamer, polypeptide, or peptide, binds to substantially the same binding site on factor B as an antibody described in U.S. Pat. Pub. No. 20050260198 or WO/2009/029669. In some embodiments, the complement inhibitor comprises the monoclonal antibody fragment known as TA106 (formerly under development by Taligen Therapeutics), or antibody, small molecule, aptamer, polypeptide, or peptide, binds to substantially the same binding site on factor B as TA106 is used. In some embodiments, a peptide that binds to and inhibits factor B is identified using, for example, a method such as phage display. In some embodiments, a complement inhibitor comprises an aptamer that binds to and inhibits factor B. In some embodiments, an RNAi agent that inhibits expression of factor B may be used.

Compounds that Inhibit Factor D Activity

In certain embodiments the complement inhibitor inhibits factor D. For example, the complement inhibitor may bind to factor D. Exemplary agents include antibodies (e.g., monoclonal antibodies), antibody fragments, peptides, small molecules, and aptamers. Exemplary antibodies that inhibit factor D are described in U.S. Pat. No. 7,112,327. In certain embodiments the complement inhibitor is an antibody, small molecule, aptamer, or polypeptide that binds to substantially the same binding site on factor D as an antibody described in U.S. Pat. No. 7,112,327. FCFD4514S (formerly under development by Tanox as TNX-234, now under development for by Genentech as lampalizumab), is a humanized monoclonal antibody fragment that binds Factor D. In certain embodiments the complement inhibitor comprises FCFD4514S or an antibody, small molecule, aptamer, or polypeptide that binds to substantially the same binding site on factor D as FCFD4514S. Exemplary polypeptides that inhibit alternative pathway activation and are believed to inhibit factor D are disclosed in U.S. Pub. No. 20040038869. In some embodiments peptides that bind to and inhibit factor D, which may be identified using methods such as phage display, or aptamers that bind to and inhibit factor D, which may be identified using methods such as SELEX, may be used. In some embodiments, an RNAi agent that inhibits expression of factor D may be used.

Mammalian Complement Regulatory Proteins and Complement Receptors

In some embodiments the complement inhibitor comprises at least a portion of a mammalian, e.g., human, complement regulatory protein or complement receptor. Examples of complement regulatory proteins include, e.g., CFH, CFH related proteins (such as CFHR1), CFI, CR1, DAF, MCP, CD59, C4 bp, and complement receptor 2 inhibitor trispanning (CRIT; Inal, J., et al, J Immunol., 174(1):356-66, 2005). In some embodiments the complement regulatory polypeptide is one that is normally membrane-bound in its naturally occurring state. In some embodiments of the invention a fragment of such polypeptide that lacks some or all of a transmembrane and/or intracellular domain is used. Soluble forms of complement receptor 1 (sCRI), or soluble portions of other complement receptors, for example, are of use in certain embodiments. For example the compounds known as TP10 or TP20 (Avant Therapeutics) can be used. In some embodiments a soluble complement control protein, e.g., CFH or a CFH related protein, is used. In some embodiments the complement inhibitor is a C3b/C4b Complement Receptor-like molecule such as those described in U.S. Pat. Pub. No. 20020192758. Variants and fragments of mammalian complement regulatory proteins or receptors that retain complement inhibiting activity can be used in certain embodiments. In some embodiments an engineered polypeptide derived in part from CHF may be used, such as those described in WO2013142362, e.g., comprising a first domain comprising CCP modules 1-4, which mediates C3b binding and exerts regulatory activities, and a second domain comprising CCP modules 19-20, which enhances binding to C3b and allows recognition of self-surfaces. The domains may be linked using a linker, such as a glycine linker.

Chimeric Complement Inhibitors

In certain embodiments of the invention the complement inhibitor comprises a chimeric polypeptide comprising a first polypeptide that inhibits complement activation, linked, e.g., covalently linked, to a second polypeptide that inhibits complement activation and/or that binds to a complement component or complement activation product. In some embodiments, at least one of the polypeptides comprises at least a portion of a mammalian complement regulatory protein. The chimeric polypeptide may contain one or more additional domains located, e.g., between the first and second polypeptides or at a terminus. For example, the first and second polypeptides can be separated by a spacer polypeptide.

In some embodiments, the first and second polypeptides each comprise at least a portion of a mammalian complement regulatory protein. In some embodiments complement inhibitor comprises at least a portion of DAF and at least a portion of MCP. Exemplary chimeric polypeptides are disclosed, e.g., in U.S. Pat. No. 5,679,546, e.g., CAB-2 (also known as MLN-2222). In some embodiments the polypeptide comprises at least 4 SCR domains of at least one mammalian complement regulatory protein or complement receptor. In some embodiments the polypeptide comprises at least 4 SCR domains of each of first and second distinct mammalian complement regulatory proteins.

In some embodiments, a chimeric polypeptide comprises at least a portion of complement receptor 1 (CR1), complement receptor 2 (CR2), complement receptor 3 (CR3), complement receptor 4 (CR4) or a variant or fragment of CR1, CR2, CR3, or CR4 that binds to one or more complement components or complement activation products such as C3b, iC3b, C3d, and/or C3dg. In some embodiments, the polypeptide comprises at least 4 SCRs, e.g., at least 4 SCRs of CR1 or CR2. For example, the polypeptide can comprise the 4 N-terminal SCRs of CR2 (e.g., residues 1-250 of the mature protein). In some embodiments the chimeric polypeptide comprises at least 4 SCR domains of a mammalian complement regulatory protein and at least 4 SCR domains of a mammalian complement receptor.

Compounds that Inhibit Properdin

In some embodiments of the invention antiproperdin antibodies (e.g., monoclonal antibodies), antibody fragment, or other anti-properdin agents are used. See, e.g., U.S. Pat. Pub. No. 20030198636 or PCT/US2008/068530 (WO/2009/110918-ANTI-PROPERDIN ANTIBODIES) for examples.

Compounds that Inhibit Components of Lectin Pathway

In some embodiments the compounds inhibit one or more components of the lectin pathway. See, e.g., WO/2007/117996) METHODS FOR TREATING CONDITIONS ASSOCIATED WITH MASP-2 DEPENDENT COMPLEMENT ACTIVATION.

Compounds that Inhibit C5 Activation or Activity

In certain embodiments the complement inhibitor inhibits activation of C5. For example, the complement inhibitor may bind to C5 and inhibit its cleavage. In some embodiments, the complement inhibitor inhibits physical interaction of C5 with C5 convertase by, e.g., binding to C5 or C5 convertase or to C5 at a site that would ordinarily participate in such physical interaction. Exemplary agents that inhibit C5 activation include antibodies (e.g., monoclonal antibodies), antibody fragments, polypeptides, small molecules, and aptamers. Exemplary compounds, e.g., antibodies, that bind to C5 are described, for example, in U.S. Pat. No. 6,534,058; PCT/US95/05688 (WO 1995/029697), PCT/EP2010/007197 (WO2011063980); U.S. Pat. Pub. No. 20050090448; and U.S. Pat. Pub. No. 20060115476. U.S. Pat. Pub. No. 20060105980 discloses aptamers that bind to and inhibit C5. In some embodiments, a humanized anti-C5 monoclonal antibody, e.g., eculizumab (also known as h5G1.1-mAb; Soliris®) (Alexion), or a fragment or derivative thereof that binds to C5. In some embodiments, an antibody comprising at least some of the same complementarity determining regions (CDR1, CDR2 and/or CDR3), e.g., all of CDR1, CDR2, and CDR3, as those of eculizumab's heavy chain and/or light chain is used. In some embodiments, the antibody comprises at least some of the same framework regions as eculizumab. In some embodiments, an antibody that binds to substantially the same binding site on C5 as eculizumab is used. In some embodiments, pexelizumab (also known as h5G1.1-scFv), a humanized, recombinant, single-chain antibody derived from h5G1.1-mAb, is used. In certain embodiments the complement inhibitor comprises a Staphylococcus SSL7 protein from Staphylococcus aureus or a variant or derivative or mimetic of such protein that can bind to C5 and inhibit its cleavage.

As noted above, bispecific or multispecific antibodies can be used. For example, PCT/US2010/039448 (WO/2010/151526) discloses bispecific antibodies described as binding to two or more different proteins, wherein at least two of the proteins are selected from C5a, C5b, a cellular receptor for C5a (e.g., C5aR1 or C5L2), the C5b-9 complex, and a component or intermediate of terminal complement such as C5b-6, C5b-7, or C5b-8. In some embodiments an RNAi agent that inhibits expression of C5 or CSaR may be used.

In some embodiments, a complement inhibitor known as OmCI, or a variant, derivative, or mimetic thereof, is used. OmCI binds to C5 and inhibits its activation most likely by inhibiting interaction with convertase. OmCI is naturally produced by the tick Ornithodoros moubata. See, e.g., PCT/GB2004/002341 (WO/2004/106369) and PCT/GB2010/000213 (WO/2010/100396), for description of OmCI and certain variants thereof. It has been shown that OmCI binds to eicosanoids, in particular leukotriene (LKs), e.g., LTB4. In some embodiments, an OmCI polypeptide (or a variant, derivative, or fragment thereof) that retains the capacity to binds to a LK, e.g., LTB4, is used. In some embodiments, an OmCI polypeptide (or a variant, derivative, or fragment thereof) that has reduced capacity or substantially lacks capacity to bind to a LK, e.g., LTB4, is used.

In some embodiments the agent is an antagonist of a C5a receptor (C5aR). In some embodiments, the C5aR antagonist comprises a peptide. Exemplary C5a receptor antagonists include a variety of small cyclic or acyclic peptides such as those described in March, D R, et al., Mol. Pharmacol., 65(4), 2004, and in Woodruff, T M, et al., J Pharmacol Exp Ther., 314(2):811-7, 2005, U.S. Pat. No. 6,821,950; U.S. Ser. No. 11/375,587; and/or PCT/US06/08960 (WO2006/099330), or a mimetic thereof. In certain embodiments the complement inhibitor binds to C5aR and inhibits binding of C5a thereto. In certain embodiments a cyclic peptide comprising the sequence [OPdChaWR] (SEQ ID NO: 59) is used. In certain embodiments a cyclic peptide comprising the sequence [KPdChaWR] (SEQ ID NO: 60) is used. In certain embodiments a peptide comprising the sequence (Xaa)ₙ[OPdChaWR] (SEQ ID NO: 61) is used, wherein Xaa is an amino acid residue and n is between 1 and 5. In certain embodiments a peptide comprising the sequence (Xaa)ₙ[KPdChaWR] (SEQ ID NO: 62) is used, wherein Xaa is an amino acid residue and n is between 1 and 5. In certain embodiments n is 1. In certain embodiments n is 1 and Xaa is a standard or nonstandard aromatic amino acid. For example, the peptides F-[OPdChaWR] (SEQ ID NO: 63), F-[KPdChaWR] (SEQ ID NO: 64); Cin-[OPdChaWR] (SEQ ID NO: 65), and HCin-[OPdChaWR] (SEQ ID NO: 66) are of use in certain embodiments. Optionally the free terminus comprises a blocking moiety, e.g., the terminal amino acid is acetylated. For example, in some embodiments the C5aR antagonist is AcF-[OPdChaWR] (SEQ ID NO: 67) (also known as PMX-53). (Abbreviations: 0: ornithine; Cha: cyclohexylalanine; Cin: cinnamoyl; Hein: hydrocinnamoyl; square brackets denote internal peptide bond). In some embodiments, a C5aR antagonist comprises a compound, e.g., a peptide, disclosed in U.S. Pat. Pub. No. 20060183883 (U.S. Ser. No. 10/564,788), e.g., a compound as represented therein by formula I, formula II, formula IV, formula V, or formula VI. An exemplary C5aR antagonist is the peptide known as JPE-1375 (Jerini A G, Germany).

In some embodiments, a C5aR antagonist is a small molecule. Various small molecule C5aR antagonists are disclosed in the following references: PCT/US2005/015897 (WO/2005/110416; 4,5-DISUBSTITUTED-2-ARYL PYRIMIDINES); PCT/EP2006/005141 (WO2006128670); PCT/US2008/072902 (WO/2009/023669; SUBSTITUTED 5,6,7,8-TETRAHYDROQUINOLINE DERIVATIVES); PCT/US2009/068941 (WO/2010/075257; CSAR ANTAGONISTS). An exemplary small molecule C5aR antagonist is CCX168 (ChemoCentryx, Mountain View, Calif.).

In certain embodiments the complement inhibitor is an agent, e.g., an antibody, small molecule, aptamer, or polypeptide, that binds to substantially the same binding site on C5 or C5aR as a compound described in any of the afore-mentioned references disclosing agents that bind to C5 or C5aR. In some embodiments the complement inhibitor is not an antagonist of a C5a receptor.

Multimodal Complement Inhibitors

In certain embodiments of the invention the complement inhibitor binds to more than one complement protein and/or inhibits more than one step in a complement activation pathway. Such complement inhibitors are referred to herein as “multimodal”. In certain embodiments of the invention the complement inhibitor comprises a virus complement control protein (VCCP). The invention contemplates use of any of the agents described in U.S. Ser. No. 11/247,886 and PCT/US2005/36547. Poxviruses and herpesviruses are families of large, complex viruses with a linear double-stranded DNA genome. Certain of these viruses encode immunomodulatory proteins that are believed to play a role in pathogenesis by subverting one or more aspects of the normal immune response and/or fostering development of a more favorable environment in the host organism (Kotwal, G J, Immunology Today, 21(5), 242-248, 2000). Among these are VCCPs. Poxvirus complement control proteins are members of the complement control protein (CCP) superfamily and typically contain 4 SCR modules. In certain embodiments the VCCP is a poxvirus complement control protein (PVCCP). The PVCCP can comprise a sequence encoded by, e.g., vaccinia virus, variola major virus, variola minor virus, cowpox virus, monkeypox virus, ectromelia virus, rabbitpox virus, myxoma virus, Yaba-like disease virus, or swinepox virus. In other embodiments the VCCP is a herpesvirus complement control protein (HVCCP). The HVCCP can comprise a sequence encoded by a Macaca fuscata rhadinovirus, cercopithecine herpesvirus 17, or human herpes virus 8. In other embodiments the HVCCP comprises a sequence encoded by herpes simplex virus saimiri ORF 4 or ORF 15 (Albrecht, J C. & Fleckenstein, B., J. Virol., 66, 3937-3940, 1992; Albrecht, J., et al., Virology, 190, 527-530, 1992).

The VCCP may inhibit the classical complement pathway, the alternate complement pathway, the lectin pathway, or any two or more of these. In certain embodiments of the invention the VCCP, e.g., a PVCCP, binds to C3b, C4b, or both. In certain embodiments of the invention the PVCCP comprises one or more putative heparin binding sites (K/R—X-K/R) and/or possesses an overall positive charge. In some embodiments the PVCCP comprises at least 3 SCR modules (e.g., modules 1-3), e.g., 4 SCR modules. The PVCCP protein can be a precursor of a mature PVCCP (i.e., can include a signal sequence that is normally cleaved off when the protein is expressed in virus-infected cells) or can be a mature form (i.e., lacking the signal sequence).

Vaccinia complement control protein (VCP) is a virus-encoded protein secreted from vaccinia infected cells. VCP is 244 amino acids in length, contains 4 SCRs, and is naturally produced by intracellular cleavage of a 263 amino acid precursor. VCP runs as an ˜35 kD protein in a 12% SDS/polyacrylamide gel under reducing conditions and has a predicted molecular mass of about 28.6 kD. VCP is described in U.S. Pat. Nos. 5,157,110 and 6,140,472, and in Kotwal, G K, et al., Nature, 355, 176-178, 1988. FIGS. 3A and 3B of U.S. Ser. No. 11/247,886 and PCT/US2005/36547 (WO2006042252) show the sequence of the precursor and mature VCP proteins, respectively. VCP has been shown to inhibit the classical pathway of complement activation via its ability to bind to C3 and C4 and act as a cofactor for factor I mediated cleavage of these components as well as promoting decay of existing convertase (Kotwal, G K, et al., Science, 250, 827-830, 1990; McKenzie et al., J. Infect. Dis., 1566, 1245-1250, 1992). It has also been shown to inhibit the alternative pathway by causing cleavage of C3b into iC3b and thereby preventing the formation of the alternative pathway C3 convertase (Sahu, A, et al., J. Immunol., 160, 5596-5604, 1998). VCP thus blocks complement activation at multiple steps and reduces levels of the proinflammatory chemotactic factors C3a, C4a, and C5a.

VCP also possesses the ability to strongly bind heparin in addition to heparan sulfate proteoglycans. VCP contains two putative heparin binding sites located in modules 1 and 4 (Jha, P and Kotwal, G J, and references therein). VCP is able to bind to the surface of endothelial cells, possibly via interaction with heparin and/or heparan sulfate at the cell surface, resulting in decreased antibody binding (Smith, S A, et al., J. Virol., 74(12), 5659-5666, 2000). VCP can be taken up by mast cells and possibly persist in tissue for lengthy periods of time, thereby potentially prolonging its activity (Kotwal, G J, et al., In G P. Talwat, et al. (eds), 10^{th} International Congress of Immunology., Monduzzi Editore, Bologna, Italy, 1998). In addition, VCP can reduce chemotactic migration of leukocytes by blocking chemokine binding (Reynolds, D, et al., in S. Jameel and L. Villareal (ed., Advances in animal virology. Oxford and IBN Publishing, New Delhi, India, 1999). VCP and other PVCCPs have a relatively small size relative to mammalian CCPs, which is advantageous for delivery in the present invention.

Variola virus major and minor encode proteins that are highly homologous to VCP and are referred to as smallpox inhibitor of complement enzymes (SPICE) (Rosengard, A M, et al., Proc. Natl. Acad. Sci., 99(13), 8803-8813. U.S. Pat. No. 6,551,595). SPICE from various variola strains sequenced to date differs from VCP by about 5% (e.g., about 11 amino acid differences). Similarly to VCP, SPICE binds to C3b and C4b and causes their degradation, acting as a cofactor for factor I. However, SPICE degrades C3b approximately 100 times as fast as VCP and degrades C4b approximately 6 times as fast as VCP. The amino acid sequence of SPICE is presented in FIG. 6 (SEQ ID NO: 12) of U.S. Ser. No. 11/247,886 and PCT/US2005/36547 (WO2006042252) and can be described as follows. Referring to FIG. 6 of U.S. Ser. No. 11/247,886 and PCT/US2005/36547 (WO2006042252), a signal sequence extends from amino acid 1 to about amino acid 19. Four SCRs extend from about amino acid 20 to amino acid 263. Each SCR is characterized by four cysteine residues. The four cysteine residues form two disulfide bonds in the expressed protein. The boundaries of each SCR are best defined by the first and fourth cysteine residues in the sequence that forms the disulfide bonds of the SCR. An invariant tryptophan residue is present between cysteine 3 and cysteine 4 of each SCR. SCR1 extends from amino acid 20 or 21 to amino acid 81. Both residues are cysteines that may be involved in disulfide bonding. SCR2 extends from amino acid 86 to amino acid 143. SCR3 extends from amino acid 148 to amino acid 201. SCR4 extends from amino acid 206 to amino acid 261. The SCRs include the complement binding locations of SPICE. SPICE or any of the portions thereof that inhibit complement activation, e.g., SPICE and SPICE-related polypeptides containing four SCRs, such as those described in U.S. Pat. No. 6,551,595, are of use in the present invention.

Complement control proteins from cowpox virus (referred to as inflammation modulatory protein, IMP) and monkeypox virus (referred to herein as monkeypox virus complement control protein, MCP) have also been identified and sequenced (Miller, C G, et al., Virology, 229, 126-133, 1997 and Uvarova, E A and Shchelkunov, S N, Virus Res., 81(1-2), 39-45, 2001). MCP differs from the other PVCCPs described herein in that it contains a truncation of the C-terminal portion of the fourth SCR.

It will be appreciated that the exact sequence of complement control proteins identified in different virus isolates may differ slightly. Such proteins fall within the scope of the present invention. Complement control proteins from any such isolate may be used, provided that the protein has not undergone a mutation that substantially abolishes its activity. Thus the sequence of a VCCP such as SPICE or VCP may differ from the exact sequences presented herein or under the accession numbers listed in Table 3. It will also be appreciated that a number of amino acid alterations, e.g., additions, deletions, or substitutions such as conservative amino acid substitutions, may be made in a typical polypeptide such as a VCCP without significantly affecting its activity, such that the resulting protein is considered equivalent to the original polypeptide. The viral polypeptides identified by accession number in Table 3 below are of use in various embodiments of the invention.

TABLE 3
Representative Viral Complement Control Proteins

| Virus | Protein | Accession | Virus Type |
|---|---|---|---|
| Variola | D12L | NP_042056 | Orthopoxvirus |
| | D15L (SPICE) | AAA69423 | Orthopoxvirus |
| Vaccinia | VCP | AAO89304 | Orthopoxvirus |
| Cowpox | CPXV034 | AAM13481 | Orthopoxvirus |
| | C17L | CAA64102 | Orthopoxvirus |
| Monkeypox | D14L | AAV84857 | Orthopoxvirus |
| Ectromelia | Complement | CAE00484 | Orthopoxvirus |
| virus | control protein | | |
| Rabbitpox | RPXV017 | AAS49730 | Orthopoxvirus |
| Macaca fuscata | JM4 | AAS99981 | Rhadinavirus |
| rhadinovirus | | | (Herpesvirus) |
| Cercopithecine | Complement | NP_570746 | Herpesvirus |
| herpesvirus 17 | binding | | |
| | protein (ORF4) | | |
| Human herpes | Complement | AAB62602 | Herpesvirus |
| virus 8 | binding | | |
| | protein (ORF4) | | |

In addition to the VCCPs described above, a number of other viral proteins exist that interfere with one or more steps in a complement pathway. These proteins are also of use in certain embodiments of the present invention. Certain of these proteins do not necessarily display clear homology to cellular complement regulators known to date. For example, HSV-1, HSV-2, VZV, PRV, BHV-1, EHV-1, and EHV-4 all encode versions of a conserved glycoprotein known as gC (Schreurs, et al., J Virol., 62, 2251-2257, 1988; Mettenleiter, et al, J. Virol., 64, 278-286; 1990; Herold, et al., J Virol., 65, 1090-1098; 1991). With the exception of VZV, the gC protein encoded by these viruses binds to C3b (Friedman, et al., Nature, 309, 633-634,1984; Huemer, et al., Virus Res., 23, 271-280, 1993) gC1 (from HSV-1) accelerates decay of the classical pathway C3 convertase and inhibits binding of properdin and C5 to C3. Purified EBV virions possess an activity that accelerates decay of the alternative pathway C3 convertase and serves as a cofactor for the complement regulatory protein factor 1 (Mold et al., J Exp Med, 168, 949-969, 1988). The foregoing proteins are referred to collectively as virus complement interfering proteins (VCIPs). By any of a variety of means, such as interfering with one or more steps of complement activation, accelerating decay of a complement component, and/or enhancing activity of a complement regulatory protein, these VCIPs are said to inhibit complement. Any of these proteins, or derivatives thereof, e.g., fragments or variants thereof, can be used as a therapeutic agent in the invention. As in the case of VCCPs, will be appreciated that the exact sequence of VCIPs identified in different virus isolates may differ slightly. Such proteins fall within the scope of the present invention.

In certain embodiments of the invention a fragment or variant of a VCCP or VCIP is locally administered to a subject. Preferred fragments and variants of a PVCCP possess at least one of the following activities: (i) ability to bind to C3, C3b, or both; (ii) ability to act as a cofactor for factor I cleavage of C3; (iii) ability to bind to C4, C4b, or both; (iv) ability to act as a cofactor for factor I cleavage of C4; (v) ability to accelerate decay of existing C3 convertase of the classical pathway, alternate pathway, or both; (vi) ability to bind heparin; (vii) ability to bind to heparan sulfate proteoglycans; (viii) ability to reduce chemotactic migration of leukocytes; (ix) ability to block chemokine (e.g, MIP-1a) binding, e.g., to the surface of a cell (e.g., a leukocyte or endothelial cell surface); (x) ability to inhibit antibody binding to class I MHC molecules; (xi) ability to inhibit the classical complement pathway; (xii) ability to inhibit the alternative complement pathway; and (xiii) ability to inhibit complement-mediated cell lysis. Preferred PVCCP fragments and variants display complement binding activity, by which is meant ability to detectably bind to one or more complement components, preferably (in the case of VCCPs) selected from the group consisting of: C3, C3b, C4, and C4b. Preferred fragments or variants of HVCCPs may also display ability to detectably bind to one or more complement components. Preferably the binding of the VCCP to the complement component is specific. It will be understood that a VCCP may be able to bind to only a single complement component or may be able to bind to more than one different complement component.

In certain embodiments of the invention the PVCCP fragment or variant comprises at least 3 SCR modules (e.g., modules 1-3), preferably 4 SCR modules. Preferably each of the SCR modules displays significant sequence identity to an SCR module found in a naturally occurring PVCCP, e.g., VCP or SPICE. Preferably the multiple SCR modules are arranged in an N to C manner so as to maximize overall identity to a naturally occurring PVCCP. If the sequence of a PVCCP fragment or variant contains an SCR domain that differs from the SCR consensus sequence at one or more positions, in certain embodiments of the invention the amino acid(s) at the one or more differing positions is identical to that found at a corresponding position in the most closely related SCR found in a naturally occurring PVCCP. In certain embodiments the PVCCP variant comprises at least one SCR module from a first PVCPP and at least one SCR module from a second PVCPP. In certain embodiments the PVCCP variant comprises at least one SCR module from a PVCCP and at least one SCR from a mammalian complement control protein (RCA protein). Any number of SCR modules, e.g., 1, 2, 3, 4, or more can come from any particular PVCCP or RCA protein in various embodiments of the invention. All such combinations and permutations are contemplated, even if not explicitly set forth herein.

Generally a fragment or variant of a naturally occurring VCCP or VCIP possesses sufficient structural similarity to its naturally occurring counterpart that it is recognized by a polyclonal antibody that recognizes the naturally occurring counterpart. In certain embodiments of the invention a fragment or variant of a VCCP possesses sufficient structural similarity to VCP or SPICE so that when its 3-dimensional structure (either actual or predicted structure) is superimposed on the structure of VCP or SPICE, the volume of overlap is at least 70%, preferably at least 80%, more preferably at least 90% of the total volume of the VCP structure. A partial or complete 3-dimensional structure of the fragment or variant may be determined by crystallizing the protein as described for VCP (Murthy, 2001). Alternately, an NMR solution structure can be generated, as performed for various VCP fragments (Wiles, A P, et al., J. Mol. Biol. 272, 253-265, 1997). A modeling program such as MODELER (Sali, A. and Blundell, T L, J. Mol. Biol., 234, 779-815, 1993), or any other modeling program, can be used to generate a predicted structure. The model can be based on the VCP structure and/or any known SCR structure. The PROSPECT-PSPP suite of programs can be used (Guo, J T, et al., Nucleic Acids Res. 32(Web Server issue):W522-5, Jul. 1, 2004). Similar methods may be used to generate a structure for SPICE.

Fragments or variants of a VCCP or VCIP may be generated by any available means, a large number of which are known in the art. For example, VCCPs, VCIPs, and fragments or variants thereof can be produced using recombinant DNA technology as described below. A VCCP or VCIP fragment may be chemically synthesized, produced using PCR amplification from a cloned VCCP or VCIP sequence, generated by a restriction digest, etc. Sequences for a VCCP variant may be generated by random mutagenesis of a VCCP sequence (e.g., using X-rays, chemical agents, or PCR-based mutagenesis), site-directed mutagenesis (e.g., using PCR or oligonucleotide-directed mutagenesis, etc. Selected amino acids can be changed or added.

While not wishing to be bound by any theory, it is likely that amino acid differences between naturally occurring PVCCPs occur at positions that are relevant in conferring differences in particular properties such as ability to bind heparin, activity level, etc. For example, VCP and SPICE differ at only 11 amino acids, but SPICE has a much higher activity as a cofactor for cleavage of C3b (e.g., cleavage occurs at a much faster rate with SPICE than with VCP). The amino acid differences are likely to be responsible for the differential activities of the two proteins. The amino acids at these positions are attractive candidates for alteration to identify variants that have yet greater activity.

Additional Complement Inhibitors

In some embodiments a complement inhibitor is a naturally occurring mammalian complement regulatory protein or a fragment or derivative thereof. For example, the complement regulatory protein may be CR1, DAF, MCP, CFH, or CFI. In some embodiments of the invention the complement regulatory polypeptide is one that is normally membrane-bound in its naturally occurring state. In some embodiments of the invention a fragment of such polypeptide that lacks some or all of a transmembrane and/or intracellular domain is used. Soluble forms of complement receptor 1 (sCR1), for example, are of use in the invention. For example the compounds known as TP10 or TP20 (Avant Therapeutics) can be used. C1 inhibitor (C1-INH) is also of use. In some embodiments a soluble complement control protein, e.g., CFH, is used. In some embodiments of the invention the polypeptide is modified to increase its solubility.

In some embodiments, a complement inhibitor is a CI s inhibitor. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 68) or KTISKAKGQPREPQVYT (SEQ ID NO: 69) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

Bifunctional Complement Inhibitors

In some embodiments a complement inhibitor is a bifunctional complement inhibitor comprising a first moiety that inhibits a first complement component and a second moiety that inhibits a second complement component. In some embodiments the first moiety inhibits C3 and a second moiety that inhibits the C5a receptor. In some embodiments of the invention the first moiety inhibits C3 and a second moiety that inhibits the C3a receptor. In some embodiments of the invention the first moiety inhibits C3 and a second moiety that inhibits the C5a receptor.

### V. Complement System Biomarkers and Assays Thereof

The term “biomarker” refers to any substance that can be detected, measured, or otherwise characterized as to one or more properties and serves as an indicator of the presence or state of a particular biological property, process, pathway, condition, or response. The term “biomarker” encompasses, without limitation, proteins, nucleic acids (including protein or nucleic acid variants such as those associated with a polymorphism or mutation, modifications, subunits, fragments, complexes, cleavage products, degradation products), metabolites (e.g., small molecule metabolites), and other analytes that can be detected, measured, or otherwise observed in a sample obtained from a subject or in vivo. A biomarker may be a cell type, the presence or abundance of which may be detected or measured based on one or more of the afore-mentioned biomarkers. The term “complement system biomarker” refers to any substance that can be detected, measured, or otherwise characterized as to one or more properties and serves as an indicator of the state of the complement system or of any one or more complement components, as an indicator of a normal or abnormal (e.g., pathologic or pathogenic) process involving the complement system, and/or as an indicator of a pharmacologic response to a therapeutic intervention involving administration of an agent that modulates (e.g., inhibits or enhances) complement activation or expression or activity of one or more complement components or is intended to treat a complement-mediated disorder. A complement system biomarker may be a nucleic acid or protein whose sequence, expression, or activity correlates with the risk of a complement-mediated disorder or correlates with progression of a complement-mediated disorder in subject who develop the disorder, or with the susceptibility of the disorder to a given treatment. In certain embodiments a complement system biomarker is a genetic marker.

An “assay of a biomarker” refers to any test or procedure that detects, measures, or otherwise characterizes or yields data (information) about the biomarker. A “result” or “results” of an assay refer to data acquired through performing the assay. It will be understood that data may be raw data or may be processed in any of a variety of ways. In some embodiments an interpretation or assessment of data, which may include its implication or significance in regard to the subject's risk of a complement-mediated disorder, may be used or provided in addition to or instead of the underlying data. Something is considered to be “determined based on” an assay if data acquired through performing the assay (whether raw data, processed data, or both) and/or an interpretation or assessment of the data are used in establishing, calculating, specifying, or concluding that thing. Where the term “determined based on” is used herein, embodiments are provided in which the thing is determined in part by whatever it is described as being determined based on and embodiments in which the thing is determined based solely on that thing. The individual or entity that makes the determination may or may not be the same individual or entity that performed the assay. In some embodiments, methods described herein may comprise performing or ordering an assay that detects, measures, or otherwise characterizes or yields data about a complement system biomarker or acquiring results of such an assay or test (which may have been performed or ordered for other purposes). In some embodiments, methods described herein may comprise interpreting or assessing the data or results from an assay of a complement system biomarker in terms of their implications in regard to whether the subject is an appropriate candidate for treatment with an immune checkpoint inhibitor, or both. Where the disclosure refers to results that “indicate” or “do not indicate” something, such as (i) the likelihood that a subject has or will develop a complement-mediated disorder, (ii) whether or not the subject has an increased or decreased susceptibility to a complement-mediated disorder, or (iii) whether or not a patient has responded to treatment, it should be understood as meaning that the results indicate or do not indicate that thing to one of ordinary skill in the art who has read the disclosure, who would be capable of understanding and interpreting or assessing the results, and does not require that the thing that is indicated or not indicated is expressly mentioned or specified as such in the results, although it may be. For example, results of a genome sequencing assay would indicate to one or ordinary skill in the art whether or not the subject has a particular allele that is known in the art to be associated with an increased or decreased susceptibility to a complement-mediated disorder. For purposes hereof, an allele that is associated with increased susceptibility to a complement-mediated disorder may be referred to as a “risk allele”. An allele that is associated with increased susceptibility to a complement-mediated disorder may be referred to as a “protective allele”.

As used herein, the term “genetic marker” refers to a DNA region at a defined location in the genome that exhibits sequence variation among individuals in a population (e.g., the human population). The different sequence variants may be referred to as “alleles”. In some embodiments the DNA sequence variation is one that occurs commonly within a population (e.g., wherein each of two or more variants is present at a frequency of at least 1.0%). Such a sequence variation may be referred to as a polymorphism. In some embodiments the DNA sequence variation is one in which the more common sequence variant has a frequency of more than 99.0%. A genetic marker may be a single base-pair variation (also called a single nucleotide variation or SNV), e.g., a substitution, insertion, or deletion of a single nucleotide, or a longer one, such as an insertion or deletion of multiple nucleotides, e.g., an at least partial duplication or deletion of a gene, or a rearrangement such as a gene fusion. In some embodiments a genetic marker is a single nucleotide polymorphism (SNP), which is a polymorphism in which a single nucleotide—A, T, C or G—at a particular position in the genome differs among members of the population. Typically a SNP has only two alleles, each of which has a frequency of at least 1.0%. Those of ordinary skill in the art are aware of numerous genetic variations. Genetic variations >50 nucleotides in length are found in the Database of Genomic Structural Variation (http://www.ncbi.nlm.nih.gov/dbvar). The term “genotype” refers to the genetic makeup of a cell or a subject (e.g., a cancer patient) with reference to one or more specific genetic variations (e.g., SNPs), genes, or characteristics under consideration. In certain embodiments the genotype of a subject with respect to a particular genetic marker, e.g., a polymorphism, identifies the combination of two alleles the particular subject carries in his or her normal diploid cells, e.g., whether the subject is homozygous (both alleles are the same) or heterozygous (the subject's cells carry two different alleles). Thus, in certain embodiments a result of an assay of a complement system biomarker is the genotype of a subject with respect to a polymorphism that is in or near a complement-related gene. As used herein, the phrase “in a complement-related gene” means within a portion of the gene that is transcribed or within a regulatory region of the gene, such as a promoter. As used herein, the phrase “near a complement-related gene” means within 150 kB of the known or predicted 5′ or 3′ end of the portion of the gene that is transcribed or within 150 kB of the known or predicted 5′ or 3′ end of an identified regulatory region of the gene, e.g., the promoter. In some embodiments, a location near a complement-related gene is within 10 kB, 20 kB, 50 kB, 75, or 100 kB of the known or predicted 5′ or 3′ end of the portion of the gene that is transcribed or within 10 kB, 20 kB, 50 kB, 75 kB, or 100 kB of the known or predicted 5′ or 3′ end of an identified regulatory region of the gene, e.g., a promoter. Whether a particular genomic location is in or near a complement-related gene may be determined by reference to the human genome sequence, February 2009 Genome Reference Consortium Human Build 37 (GRCh37), also known as hg19 available at the UCSC Genome Browser website (University of California, Santa Cruz) GenBank Assembly ID: GCA_000001405.1; RefSeq Assembly ID: GCF_000001405.13. In certain embodiments a polymorphism is genetically linked to a polymorphism that is in or near a complement-related gene. Such a polymorphism may be used instead of or in addition to the polymorphism with which it is genetically linked.

In some aspects, described herein is a method of treating a subject in need of treatment for cancer comprising: (a) obtaining the genotype of the subject with respect to a polymorphism or mutation in or near a complement-related gene, wherein at least one allele of the polymorphism or mutation is a risk allele for AMD; and (b) treating the subject with an immune checkpoint inhibitor and a complement inhibitor if the subject is homozygous or heterozygous for the risk allele. In some embodiments the gene encodes a complement protein. In some embodiments the gene encodes a complement control protein. In some embodiments step (a) comprises obtaining the genotype with respect to two or more polymorphisms or mutations (or at least one polymorphism and at least one mutation) each of which is in or near a complement-related gene. In some embodiments the polymorphisms or mutations are in different complement-related genes or located closest to different complement-related genes. In some embodiments the polymorphism or mutation is in or near the gene that encodes CFH. In some embodiments the polymorphism or mutation is in or near the gene that encodes CFI. In some embodiments the polymorphism or mutation is in or near the gene that encodes C3. In some embodiments the risk allele is a risk allele for developing geographic atrophy (GA), an advanced form of dry AMD. In some embodiments the risk allele is a risk allele for rapid increase in area of GA lesions. For example, subjects having the allele may have more rapid increase in area of GA lesions as compared to subjects with GA who do not have the risk allele. In some embodiments the risk allele is a risk allele for rapid increase in area of GA lesions is in or near the CFI gene.

In some aspects, described herein is a method of treating a subject in need of treatment for cancer comprising: (a) obtaining the genotype of the subject with respect to a polymorphism or mutation in or near a complement-related gene, wherein at least one allele of the polymorphism or mutation is a risk allele for developing AMD; and (b) treating the subject with an immune checkpoint inhibitor and a complement inhibitor if the subject is homozygous or heterozygous for the risk allele. In some embodiments the gene encodes a complement protein. In some embodiments step (a) comprises obtaining the genotype with respect to two or more polymorphisms or mutations (or at least one polymorphism and at least one mutation) each of which is in or near a complement-related gene. In some embodiments the polymorphisms or mutations are in different complement-related genes or located closest to different complement-related genes. In some embodiments the gene encodes CFH. In some embodiments the gene encodes C3. In some embodiments the polymorphism or mutation is in or near the gene that encodes CFH. In some embodiments the polymorphism or mutation is in or near the gene that encodes CFI. In some embodiments the polymorphism or mutation is in or near the gene that encodes C3.

In some aspects, described herein is a method of selecting a subject in need of treatment for cancer as a suitable candidate for therapy with an immune checkpoint inhibitor and a complement inhibitor, the method comprising: (a) obtaining the genotype of the subject with respect to one or more polymorphisms or mutations, wherein at least one variant of such polymorphism or mutation is associated with increased risk for developing a complement-mediated disorder; and (b) selecting the subject as a suitable candidate for therapy with an immune checkpoint inhibitor and a complement inhibitor based on the genotype. In some embodiments the subject is selected as a suitable candidate if the subject is homozygous or heterozygous for the variant associated with the increased risk. In some embodiments the subject is selected as a suitable candidate if the subject is not homozygous or heterozygous for the variant associated with the increased risk. In some embodiments the polymorphism or mutation is in or near or genetically linked to a gene that encodes a complement protein. In some embodiments the polymorphism or mutation is in or near or genetically linked to a gene that encodes a complement control protein. In some embodiments the method further comprises treating the patient with an immune checkpoint inhibitor and a complement inhibitor. In some embodiments the complement-mediated disorder is AMD. In some embodiments the polymorphism or mutation is in or near the gene that encodes CFH. In some embodiments the polymorphism or mutation is in or near the gene that encodes CFI. In some embodiments the polymorphism is in or near the gene that encodes C3. In some embodiments step (a) comprises obtaining the genotype with respect to two or more polymorphisms or mutations, each of which is in or near a complement-related gene. In some embodiments the polymorphisms or mutations are in different complement-related genes or located closest to different complement-related genes, e.g., CFH and C3, CFH and CFI, CFI and C3, or all of CFH, CFI, and C3.

In some embodiments a polymorphism of interest herein has a minor allele frequency of at least 5%, at least 10%, at least 20%, or at least 30%. In some embodiments a method comprises determining the genotype of the subject with respect to 1, 2, 3, 4, or more polymorphisms, each having a minor allele frequency of at least 5%, at least 10%, at least 20%, or at least 30%.

Certain genetic variants of interest herein are alleles of the gene that encodes complement factor H (CFH). In some embodiments the alleles encode a CFH isoform that contains His rather than Tyr at position 402 (Tyr402His), e.g., resulting from a T to C substitution at nucleotide 1277 in exon 9 of the CFH gene (T1277C). The Tyr402His variant of CFH is associated with a considerably increased risk of AMD. Without wishing to be bound by any theory, the Tyr402His variant of CFH may be less effective at controlling complement activation and/or may have altered tissue localization, adversely affecting its complement control ability. Other CFH isoforms are also tightly associated with AMD risk (Klein, R. J. et al. Complement Factor H Polymorphism in Age-Related Macular Degeneration. Science (2005); 308:385-9; Edwards, A. O. et al. Complement Factor H Polymorphism and Age-Related Macular Degeneration. Science (2005); 308:421-4; Haines, J. L. et al. Complement Factor H Variant Increases the Risk of Age-Related Macular Degeneration. Science (2005); 308(5720):419-21; Narayanan R, et al., Ophthalmology. (2007); 114(7):1327-31. Variants of the genes that encode complement proteins C2, C3, factor B, C7 and MBL-2 have also been associated with AMD risk (Gold, B. et al. Variation in factor B (BF) and complement component 2 (C2) genes is associated with age-related macular degeneration. Nat. Genet. (2006); 38, 458-462; Dinu, V. et al. Evidence for Association between Multiple Complement Pathway Genes and AMD. Genet. Epidem. (2007); 31, 224-237; Yates, J. R. W., Complement C3 Variant and the Risk of Age-Related Macular Degeneration, N. Engl. J. Med. (2007); 357: 19-27.

In some embodiments the polymorphism in or near the CFH gene may be any of the following, wherein the risk allele for AMD is indicated.

TABLE 1A

| | |
|---|---|
| rs3753394 | T |
| rs7524776 | C |
| rs551397 | G |
| rs800292 | C |
| rs1329424 | A |
| rs1061170 | C |
| rs10801555 | A |
| rs12124794 | A |
| rs6695321 | A |
| rs10733086 | A |
| rs10737680 | A |
| rs2274700 | C |
| rs3753396 | G |
| rs1410996 | C |
| rs380390 | C |
| rs10737680 | A |
| rs1329424 | T |
| rs1329428 | G |

In some embodiments the polymorphism in CFH is rs6677604, wherein the minor allele of rs6677604 (A) is the risk allele for AMD.

Certain genetic variants of interest herein are alleles of the gene that encodes C3. Variations in or near the gene that encodes C3 are also associated with risk of AMD (Thakkinstian A, et al. Am J Epidemiol. 2011; 173(12):1365-1379. For example, R102G (rs2230199) is a common risk variant (Maller J B, et al. Nature Genetics. 2007; 39:1200-1201). The K155Q variant of C3 (SNP rs147859257) and R1210C variant of C3 are also associated with increased risk of AMD. The K155Q allele in C3 results in resistance of the C3 protein to proteolytic inactivation by CFH and CFI (Seddon, J., et al. Nat Genet. 2013; 45(11): 1366-1370). In some embodiments, the polymorphism is one that results in versions of the C3 protein that correspond to the electrophoretic variants C3S (slow) and C3F (fast). For example, one such polymorphism is the SNP rs2230199, wherein the allele encoding a C3 protein having a Gly at position 80 (Arg80Gly) corresponds to the F variant and is the risk allele for AMD. Without being bound by any theory, the C3F variant of C3 (e.g., a variant having a Gly at position 80) may be associated with increased risk of nonresponsiveness to treatment with an immune checkpoint inhibitor.

In some embodiments the polymorphism in or near the C3 gene may be any of the following, wherein the risk allele for AMD is indicated.

TABLE 1B

| | |
|---|---|
| rs11569562 | C |
| rs7257062 | T |
| rs408290 | C |
| rs8112351 | C |
| rs2230205 | A |
| rs1047286 | T |
| rs2230199 | G |
| rs2250656 | A |

Certain genetic variants of interest herein are alleles of the gene that encodes CFI. CFI is a two-chain serine protease in which the light chain carries the catalytic domain. CFI downregulates the alternative and classical complement pathways by cleaving the alpha chains of C3b and C4b in the presence of cofactor proteins. Mutations 1322T, D501N and D506V reside in the serine protease domain of CFI and result in secreted proteins that lack C3b and C4b cofactor activity. The del TTCAC (1446-1450) mutant leads to a protein that is not secreted. The R299W mutant lies in a region of the CFI heavy chain of no known function (Kavanagh, D., Characterization of mutations in complement factor I (CFI) associated with hemolytic uremic syndrome; Mol Immunol; 45(1), pp. 95-195, 2008). Other CFI mutations of interest are found in the CFI mutation database (CFIbase) at the website having URL: structure.bmc.lu.se/idbase/, which includes at least 30 CFI mutations found in aHUS. Variation in or near the CFI gene is also associated with susceptibility to AMD (Fagerness J A, et al. Eur. J. Hum. Genet. 2009; 17:100-104; Ennis, S., et al., Eur. J. Hum. Genet. (2010) 18, 15-16). For example, alleles of rs10033900 (CIT, of which T allele is risk allele), rs13117504 (C/G, of which C allele is risk allele), rs11726949 (C/T, of which T is the risk allele), rs11728699 (G/T, of which T is the risk allele), rs7439493 (G/A, of which G is the risk allele) and rs11728699 (G/T, of which T is the risk allele) are associated with increased susceptibility to AMD. Also of interest is rs4698775 (T/G), which is near the CFI gene and of which the minor allele (G) is associated with increased AMD susceptibility (Fritsche L G, et al., Nat Genet. (2013); 45(4):433-9, 439e1-2). In some embodiments rs541862 may be used, e.g., as a proxy SNP for rs4698775. In some embodiments rs17440077 may be used, e.g., as a proxy SNP for rs4698775. rs17440077 is located near the CFI gene, in an intron region of the CCDC109B gene (coiled-coil domain containing 109B) and is in strong linkage disequilbrium with rs4698775. A rare, highly penetrant missense mutation in CFI encoding a Gly119Arg substitution confers high risk of AMD (van de Ven, et al. Nat Genet. 2013; 45(7):813-7) CFI variants that have been found in patients with atypical hemolytic uremic syndrome and also identified in a study of rare variants in CFI, C3 and C9 associated with high risk of advanced AMD include P50A15, G119R15, A240G16, G261D17, R317W16, I340T18, Y369S16,19, D403N15, I416L19, Y459S15, R474X20, and P553S (Seddon, J., et al. Nat Genet. 2013; 45(11): 1366-1370). In some embodiments the polymorphism in or near the CFI gene may be any of the following, wherein the risk allele for AMD is indicated.

TABLE 1C

| | |
|---|---|
| rs10033900 | T |
| rs13117504 | C |
| rs11726949 | T |
| rs11728699 | T |
| rs7439493 | G |
| rs4698775 | G |
| rs17440077 | G |
| rs2285714 | T |
| rs10033900 | T |

Certain genetic variants of interest herein are alleles of the gene that encodes C9 The P167S allele in the gene that encodes C9 (rs34882957) is associated with increased AMD susceptibility (Seddon, 2013).

Certain alleles of various complement-related genes have been associated with a reduced risk of developing AMD relative to other alleles. It will be appreciated that in such instances, having the protective allele(s) decreases risk of developing AMD. In some embodiments described herein, having such a protective allele increases the likelihood that a subject will respond to treatment with an immune checkpoint inhibitor. In some embodiments described herein, not having such a protective allele increases the likelihood that a subject will not respond to treatment with an immune checkpoint inhibitor. In some embodiments, at least one of the polymorphisms is in the CFB or C2 gene, for example the polymorphisms disclosed as being linked to protection from AMD in PCT/US2006/003904, PCT/US2006/003696, and/or PCT/US2007/061964. Without limitation, the method may comprise determining whether a subject has any one or more of the following: (i) A or G at rs641153 of the CFB gene, or R or Q at position 32 of the CFB protein; (ii) A or T at rs4151667 of the CFB gene, or L or H at position 9 of the CFB protein; (iii) G or T at rs547154 of the C2 gene or; and (iv) C or G at rs9332739 of the C2 gene, or E or D at position 318 of the C2 protein. For the C2 polymorphisms, the minor allele Cat rs9332739 and the minor allele T at rs547154 carry reduced risk of AMD. For the CFB polymorphisms, the minor alleles A at rs4151667 and rs614153 carry reduced risk of AMD. See also Thakkinstian, A. et al., American Journal of Epidemiology, 176(5): 361-372.

In some embodiments, the method comprises assessing a subject's genotype at the CBF or C2 locus and/or at the CFH locus with respect to whether the subject is: (i) heterozygous for the R32Q polymorphism in CFB; (ii) heterozygous for the L9H polymorphism in CFB; (iii) heterozygous for the IVS 10 polymorphism in C2; (iv) heterozygous for the E318D polymorphism in C2; (v) homozygous for the delTT polymorphism in CFH; and/or (vi) homozygous for the R15OR polymorphism in CFB and, optionally, homozygous for Y402H in CFH.

In some embodiments the polymorphism or mutation is associated with atypical hemolytic uremic syndrome (aHUS). Genetic abnormalities in complement regulatory proteins, including complement factor H (CFH), membrane cofactor protein (MCP), and complement factor 1 (CFI), and in CFHR1 and CFHR3, have been reported in 30%, 10%, and 5% of patients with atypical HUS, respectively (see, e.g., Caprioli J, et al., Blood 108: 1267-1279, 2006; Richards A, et al., Am J Hum Genet 68: 485-490, 2001; Fremeaux-Bacchi V, et al., J Am Soc Nephrol 17: 2017-2025, 2006; Zipfel, P., et al., PLOS Genetics, Vol. 3(3): 387-392, e41, March 2007. At least 127 CFH mutations have been reported in patients with aHUS (listed in the database at URL www.FH-HUS.org, The Factor H-Associated HUS Mutation Database). Most of these mutations are heterozygous missense mutations that cluster in the exons encoding SCRs 19 and 20. Functional studies on the mutant proteins have shown reduced C3b and heparin binding that results in impaired control of complement activation on the endothelial cell surface. These mutations include two nucleotide changes, c.3572C>T and c.3590T>C, that in some patients occur in combination. Also reported in The Factor H-Associated HUS Mutation Database are 30 Factor 1 mutations and 48 MCP mutations linked with HUS and 5 mutations within CFH that are associated with MPGN. These mutations are thought to lead to inability to appropriately control the complement cascade at sites of cell injury and tissue damage such as that occurring in trauma. Mutations in C3 are also found in patients with aHUS (see The Factor H-Associated HUS Mutation Database) and Bresin, E., et al. J Am Soc Nephrol. 2013; 24(3):475-86. Some mutations in CFH, such as G3587T, which introduces a stop codon at position 1172, eliminate one or more of the C-terminal short concensus repeats. This mutation severely affects recognition functions (i.e., binding to heparin, C3b, C3d, and the surface of endothelial cells). The mutant factor H protein show severely reduced regulatory activities and lack of protective function on the surface of endothelial cells, leading to defective complement control on cell surfaces (Heinen, S., et al., Hemolytic uremic syndrome: a factor H mutation (E1172Stop) causes defective complement control at the surface of endothelial cells, Am Soc Nephrol. (2007)18(2):506-14, 2007).

Mutations within complement Factor B have also been associated with aHUS (Goicoechea de Jorge et al., Proc Natl Acad Sci USA. 2007; 104(1):240-5) F286L and K323E aHUS-associated CFB mutations are gain-of-function mutations that result in enhanced formation of the C3bBb convertase or increased resistance to inactivation by complement regulators.

Certain embodiments encompass assessing the genotype of a subject with respect to any of the afore-mentioned mutations and polymorphisms associated with aHUS or MPGN, wherein a subject having one or more such mutations or risk-associated alleles of a polymorphism is identified as being at increased or decreased risk of not responding to treatment with an immune checkpoint inhibitor.

Aspects of the present disclosure encompass obtaining the genotype of a subject with respect to any of the polymorphisms or mutations described herein, or with respect to any other polymorphisms or mutations in the same genes or in other gene(s) encoding a complement-related protein or in a gene in which mutation or variation has been linked to a complement-mediated disorder and selecting the subject as a suitable candidate for treatment with an immune checkpoint inhibitor, or for treatment with an immune checkpoint inhibitor and a complement inhibitor, based on the genotype. Exemplary SNPs of interest herein include, without limitation, the following: rs1061170, rs2274700, rs1410996, rs10737680, rs7535263, rs10801559, rs3766405, rs10754199, rs1329428, rs10922104, rs1887973, rs10922105, rs4658046, rs10465586, rs3753396, rs402056, rs7529589, rs7514261, rs10922102, rs10922103, rs800290, rs1061147, rs1048663, rs412852, rs11582939, rs1280514, rs2876849, rs930508, rs2250656, rs2230203, rs2230204, rs2287846, rs344542, rs2241393, rs344550, rs2277984, rs7033790, rs17611, rs7026551, rs3753394, rs800292 (I62V), rs380390, rs3766404, rs529825 (IVS1), rs203674. Other SNPs of interest include the following (gene containing the relevant polymorphism is indicated in parentheses): rs1047286 (C3), rs2230199 (C3), rs9332739 (C2), rs547154 (C2), rs4151667 (CFB), rs641153 (CFB), rs41015361 (C7), rs33682798 (MBL2), rs930508 (MBL2).

In some embodiments, at least two polymorphisms, e.g., SNPs, are evaluated, wherein a first polymorphism is in or near the CFH gene and a second polymorphism is in or near a second locus or gene selected from the group consisting of C3, CFB, CFI, C7, and C9. In some embodiments, the second gene is the C3 gene. In some embodiments, the second gene is the CFI gene. In some embodiments, at least two polymorphisms, e.g., SNPs, are evaluated, wherein a first polymorphism is in or near the CFI gene and a second polymorphism is in or near a second locus or gene selected from the group consisting of C3, CFB, CFH, C7, and C9. In some embodiments, the second gene is the CFH gene. In some embodiments, the second gene is the C3 gene. In some embodiments at least one, two, or three SNPs is/are selected from rs2230199 (C3), rs1047286 (C3), rs1410996 (CFH), rs1061170 (CFH), rs2274700 (CFH), rs10033900 (CFI), rs13117504 (CFI), rs11726949 (CFI), rs11728699 (CFI), rs7439493 (CFI), rs11728699 (CFI), rs4698775 (near CFI), and rs17440077 (near CFI).

In some embodiments at least one SNP is selected from the SNPs listed in Table 1A. In some embodiments at least one SNP is selected from the SNPs listed in Table 1B. In some embodiments at least one SNP is selected from the SNPs listed in Table 1C. In some embodiments at least one SNP is selected from the SNPs listed in Table 1A and at least one SNP is selected from the SNPs listed in Table 1B. In some embodiments at least one SNP is selected from the SNPs listed in Table 1A and at least one SNP is selected from the SNPs listed in Table 1C. In some embodiments at least one SNP is selected from the SNPs listed in Table 1B and at least one SNP is selected from the SNPs listed in Table 1C. In some embodiments at least one SNP is selected from the SNPs listed in Table 1A, at least one SNP is selected from the SNPs listed in Table B, and at least one SNP is selected from the SNPs listed in Table 1C.

In some embodiments, at least one SNP is selected from: rs1061170, rs1047286, rs2230199, rs120862610, rs9332739, rs547154, rs4151667, rs641153, rs41015361, rs33682798, rs10490924, and rs1045216. In some embodiments at least two SNPs are selected from: rs1061170, rs1047286, rs2230199, rs120862610, rs9332739, rs547154, rs4151667, rs641153, rs41015361, rs33682798, rs10490924, and rs1045216. In some embodiments at least three SNPs are selected from: rs1061170, rs1047286, rs2230199, rs120862610, rs9332739, rs547154, rs4151667, rs641153, rs41015361, rs33682798, rs10490924, and rs1045216. In some embodiments, at least one SNP is selected from: rs1061170, rs2230199, rs10490924, rs641153, rs1047286, and rs9332739. In some embodiments at least one SNP is selected from: rs7033790, rs17611, rs120862610, rs547154, rs4151667, rs41015361, rs33682798, and rs1045216.

It will be appreciated that polymorphisms, e.g., SNPs, may be in linkage disequilibrium (LD) with other polymorphisms, e.g., other SNPs, located on the same chromosome. Such SNPs may be present in haplotypes. For example, some SNPs may be linked over distances of up to 100 kB or even 150 kB or more (Reich, D. E., et al., Nature, 411, 199-204, 2001). Thus in some embodiments a method comprises determining whether an individual has a haplotype that comprises at least one allele of a polymorphism associated with increased susceptibility to a complement-mediated disorder, wherein said polymorphism is in or near a complement-related gene. In some embodiments the haplotype comprises the Tyr402His coding variant of the CFH gene (encoded by the C allele of rs1061170). The individual may be heterozygous for the haplotype. In some embodiments the haplotype is a CFH haplotype that does not comprise the Tyr402His coding variant (see, e.g., Li, et al., Nature Genet. 38: 1049-1054, 2006). The individual may be homozygous for the haplotype.

Polymorphisms (e.g., SNPs) that are linked to the specific polymorphisms (e.g., SNPs) disclosed herein are of use in the methods in certain embodiments. As in the case of certain polymorphisms disclosed above, the frequencies of certain variant forms of a linked polymorphism of use in the methods of the invention differ between those individuals who had a poor outcome following trauma and those individuals who did not have a poor outcome following trauma and/or between those individuals who have or are susceptible to AMD or another complement-mediated disorder, and those individuals who do not have the disorder or increased susceptibility to the disorder. Therefore, one or more variant forms of a linked polymorphism may be associated with poor outcome following trauma and is of use in method of the present invention. In some embodiments the polymorphism is in strong linkage disequilibrium with any of the polymorphisms associated with risk of a complement-mediated disorder, e.g., any of the polymorphisms mentioned herein or known in the art to be associated with a complement-mediated disorder, e.g., AMD. A variety of metrics are known in the art that may be used to evaluate the extent to which any two alleles are in linkage disequilibrium (LD). Suitable metrics include D′, r², and others (see, e.g., Hedrick, P. W., Genetics, 117(2):331-41, 1987). As used herein, “strong LD” is said to exist if D′>0.8. In some embodiments at least one, two, three, or more SNPs is/are selected from rs2230199 (C3), rs1047286 (C3), rs1410996 (CFH), rs1061170 (CFH), rs2274700 (CFH), rs6677604 (CFH), rs10033900 (CFI), rs13117504 (CFI), rs11726949 (CFI), rs11728699 (CFI), rs7439493 (CFI), rs11728699 (CFI) and rs4698775 (near CFI) or a SNP in strong LD with any of the foregoing. In some embodiments the SNP has an r² with any of the SNPs mentioned herein of at least 0.8, e.g., at least 0.9. Without limitation, in some embodiments the SNP has an r² of at least 0.8, e.g., at least 0.9, with one or more of rs2230199 (C3), rs1047286 (C3), rs1410996 (CFH), rs1061170 (CFH), rs2274700 (CFH), rs6677604 (CFH), rs10033900 (CFI), rs13117504 (CFI), rs11726949 (CFI), rs11728699 (CFI), rs7439493 (CFI), rs11728699 (CFI) and rs4698775 (near CFI). For example, in some embodiments the SNP is rs17440077 (near CFI), in strong LD with rs4698775. In some embodiments the SNP is rs10922106, in strong LD with rs10737680.

It will be appreciated that mutations and polymorphisms specifically described herein are merely exemplary. Embodiments of the present disclosure encompass assessing the genotype of a subject with respect to any mutation or polymorphism that has been demonstrated to result in (or is genetically associated with), decreased ability to control the complement cascade (e.g., at sites of cell damage and/or tissue injury or on normal cells) or with increased baseline complement activation capacity, and/or has been demonstrated to be associated with risk of a complement-mediated disorder.

In some aspects, the disclosure provides a method of determining polymorphisms that are relevant to response to treatment with an immune checkpoint inhibitor comprising: (a) obtaining the sequence of at least a portion of each of one or more complement-related genes from a plurality of subjects having cancer treated with an immune checkpoint inhibitor; (b) determining nucleotide differences in or near one or more of said genes among the plurality of subjects; and (c) correlating the nucleotide differences with response to treatment with the immune checkpoint inhibitor to determine relevant polymorphisms. A polymorphism may be considered “relevant to” response to treatment with an immune checkpoint inhibitor if the genotype of a subject with respect to the polymorphism is predictive of the likelihood that the subject will respond to treatment with an immune checkpoint inhibitor, useful for classifying subjects according to predicted response, useful for selecting appropriate candidates for treatment with an immune checkpoint inhibitor, useful for selecting combination therapies to be administered with the immune checkpoint inhibitor, or other purposes described herein. In some embodiments one or more collections of data and/or samples (e.g., from clinical trials) may be used. In some embodiments, alleles that occur with a different frequency in subjects who respond to treatment than in subjects who do not respond to treatment are identified. For example, in some embodiments, alleles that occur more frequently in subjects who respond to treatment than in subjects who do not respond to treatment are identified. In some embodiments, alleles that occur with a different frequency in subjects who respond to treatment than in the overall population are identified. For example, in some embodiments, alleles that occur more frequently in subjects who respond to treatment than in the overall population are identified. Methods (e.g., statistical methods) known in the art can be used to identify and/or confirm correlations, e.g., to determine that a difference in allele frequencies between two or more groups of subjects (e.g., subjects who respond and subjects who do not respond to a treatment) is statistically significant, e.g., with a p-value of less than 0.05, less than 0.02, or less than 0.01. It will be understood that a population may be the human population in a relevant geographic area, such as a region, country, state, etc. It will also be understood that a population may be limited to individuals of Caucasian ancestry, European ancestry, Asian ancestry, African ancestry etc. It will also be understood that a characteristic of a population may be measured in a sample (where the term “sample” is used in the statistical sense rather than in the sense of a biological sample) of the population containing a suitable number of individuals. In some embodiments a sample may contain between 200 and 100,000 individuals, e.g., up to about 1,000; 5,000; 10,000, or more individuals.

Certain polymorphisms and/or mutations (or combinations thereof) that are in or near a complement-related gene may be particularly useful for classifying patients according to their likelihood of responding or not responding to treatment with particular immune checkpoint inhibitor(s) or for classifying particular cancer types according to the likelihood that a subject with a cancer of that type will respond or not respond to treatment with an immune checkpoint inhibitor. For example, certain variants and/or mutations (or combinations thereof) may have a particularly high degree of correlation with responsiveness. Additional polymorphisms and/or mutations that are associated with increased likelihood of response, or nonresponsiveness, to treatment with an immune checkpoint inhibitor and are located in or near a complement-related gene may be identified. Such additional polymorphisms and/or mutations may be used in methods of classifying, identifying, selecting, and/or treating subjects described herein.

Methods of determining polymorphisms that are relevant to response to treatment with an immune checkpoint inhibitor may comprise analyzing known polymorphisms to identify those in which the presence of a particular allele in subjects correlates with response and/or may comprise identifying new polymorphisms to identify those in which the presence of a particular allele in subjects correlates with response and/or to identify those polymorphisms that are particularly useful across cancers, for one or more particular cancer types, and/or for a particular immune checkpoint inhibitor. The portion of sequence obtained may be as little as a singe nucleotide (e.g., if analyzing a known polymorphism) up to the full length of the gene. In some embodiments the sequence comprises the transcribed portion of the gene. In some embodiments the sequence alternately or additionally comprises one or more regulatory regions, e.g., a promoter. The sequence may further comprise sequence near the gene. In some embodiments a whole genome sequence may be used. Methods of determining polymorphisms that are relevant to response to treatment with an immune checkpoint inhibitor may use biological samples collected from subjects treated with an immune checkpoint inhibitor in a clinical trial. In some embodiments archived blood or tissue samples may be used.

The genotype of a subject can be determined using any of a variety of methods. The particular method employed is not critical and need not be described here in detail, such methods being well known in the art. The methods typically utilize a biological sample obtained from the subject, wherein the biological sample (sometimes referred to simply as a “sample”) comprises nucleic acids and/or proteins. As used herein, a “biological sample” refers to any of the following: a cell or cells, a portion of tissue, a body fluid such as blood, urine, saliva, cerebrospinal fluid, etc. The term “biological sample” also includes any material derived by processing a biological sample as previously defined, e.g., by isolating or purifying DNA, RNA, and/or protein from the sample, by subjecting the sample or a portion thereof to amplification, restriction enzyme digestion, fractionation, separation, or other manipulation. In many embodiments a blood or tissue sample is used. In some embodiments DNA is isolated from white blood cells in the blood sample. Methods can involve testing the individual's DNA to determine whether the DNA comprises an allele of interest, e.g., with respect to a particular polymorphism or mutation of interest. RNA can also be used if the polymorphism or mutation of interest lies in a portion of the gene that is transcribed. In some embodiments the methods involve determining the identity of a particular nucleotide, wherein a polymorphism or mutation at the position of such nucleotide is associated with increased or decreased risk of nonresponsiveness to treatment with an immune checkpoint inhibitor and/or with increased or decreased susceptibility to AMD or another complement-mediated disorder.

Methods for performing such tests are well known in the art and include, e.g., isolating the DNA or RNA, optionally amplifying it (e.g., using the polymerase chain reaction (PCR) or reverse transcription PCR), and performing a variety of methods such as allele-specific primer extension, allele-specific hybridization, restriction enzyme digestion, sequencing, etc. In some embodiments genotyping is performed using a microarray, or “chip”, such as those available from Affymetrix or Agilent or a bead-based microarray such as those available from Illumina. In some embodiments genotyping is performed using a bead-based assay such as the Luminex platform. Other methods of use include oligonucleotide ligation assays (U.S. Pat. Nos. 5,185,243, 5,679,524 and 5,573,907), cleavage assays, heteroduplex tracking analysis (HTA) assays, etc. Examples include the Taqman™ assay, Applied Biosystems (U.S. Pat. No. 5,723,591). Cycling probe technology (CPT), which is a nucleic acid detection system based on signal or probe amplification rather than target amplification (U.S. Pat. Nos. 5,011,769, 5,403,711, 5,660,988, and 4,876,187), could also be employed. Invasive cleavage assays, e.g., Invader™ assays (Third Wave Technologies), described in Eis, P. S. et al., Nat. Biotechnol. 19:673, 2001, can also be used. Assays based on molecular beacons (U.S. Pat. Nos. 6,277,607; 6,150,097; 6,037,130) or fluorescence energy transfer (FRET) may be used. U.S. Pub. No. 20050069908 and references therein describe a variety of other methods that can be used for the detection of nucleic acids. U.S. Pat. Nos. 5,854,033, 6,143,495, and 6,239,150 describe compositions and a method for amplification of and multiplex detection of molecules of interest involving rolling circle replication. The method is useful for simultaneously detecting multiple specific nucleic acids in a sample. In some embodiments the nucleic acids are sequenced. U.S. Pub. No. 20050026180 describes methods for multiplexing nucleic acid reactions, including amplification, detection and genotyping, which can be adapted for determining the sequence at specific locations of interest for purposes of determining the genotype of an individual with respect to any genetic marker, e.g., polymorphism or mutation, of interest.

It will be understood that in various embodiments an agent of use for performing an assay of a complement system biomarker (or other assay) may, for example, comprise a detectable label (e.g., a radiolabels, enzyme labels (e.g., luciferase, horseradish peroxidase, alkaline phosphatase), or fluorescent label (e.g., a small molecule fluorophore such as an Alexa dye, fluorescein, etc.). In some embodiments an agent of use for performing an assay of a complement system biomarker (or other assay) may be associated with a solid support.

In summary, and without limitation, suitable methods include hybridization-based methods such as dynamic allele-specific hybridization, use of molecular beacons, SNP microarrays, enzyme-based methods such as those based on restriction fragment length polymorphism, PCR-based methods, methods employing flap endonuclease, primer extension, 5′-nuclease, oligonucleotide ligase assay, other post-amplification methods based on physical properties of DNA, single strand conformation polymorphism, temperature gradient gel electrophoresis, denaturing high performance liquid chromatography, and sequencing. High throughput sequencing (sometimes termed next generation sequencing) is becoming ever more efficient at a rapid pace, and it is envisioned that sequencing may routinely be used for genotyping purposes. In some embodiments an individual may have his or her entire genome or a portion thereof, e.g., exome, sequenced and stored on a non-transitory computer-readable medium. The sequence may be examined (which may be done using a suitable computer program) to determine the sequence at any location of interest, e.g., the location of a polymorphism or potential mutation of interest, or otherwise ascertain the genotype. Next generation sequencing encompasses a variety of technologies that parallelize the sequencing process, producing hundreds of thousands, millions, or billions of sequences concurrently. Such sequences (sometimes termed “reads”) may be quite short and may be assembled, e.g., by comparison with a reference genome or de novo based on aligning overlapping reads. Methods based on pyrosequencing, in situ sequencing, bead-based sequencing, sequencing by synthesis (e.g., Illumina sequencing), nanopore sequencing, sequencing by ligation (e.g., SOLiD™ sequencing), ion semiconductor sequencing (e.g., Ion Torrent™ sequencing), etc., are of use. See also PCT/US2006/029449 (WO2007014338) and US20070087362 for further information on certain sequencing approaches.

Certain methods comprise measuring the expression level of the mRNA encoding the protein or the level of the protein itself. Such methods are of use, e.g., when an allele of interest has an altered level of expression relative to other alleles. For example, a polymorphism in a regulatory region of a gene may result in increased or decreased expression. Alterations in mRNA expression can be assessed using hybridization-based approaches such as microarrays (e.g., oligonucleotide microarrays such as those available from Affymetrix, Illumina, or Agilent), Northern blot, quantitative RT-PCR, etc.), RNA sequencing (RNA-Seq). Alterations in protein expression can be assessed using, e.g., protein microarrays, Western blot, mass spectrometry, immunoassays (e.g., ELISA assays, fluorescently labeled antibodies, surface plasmon resonance-based assays), etc.

An alteration in gene expression may be caused, e.g., by a mutation or polymorphism in a coding region or non-coding region (e.g., a regulatory region, an intron) of a gene, by an increase or decrease in copy number, or by an epigenetic change (e.g., altered DNA methylation or altered histone modification (e.g., altered acetylation or methylation). Epigenetic changes can be detected, e.g., using bisulfite sequencing, ChIP-on-chip, and/or ChIP-Seq.

In some embodiments, a method of determining the genotype comprises assessing one or more characteristics of a protein encoded by a gene of interest, e.g., a complement-related gene, wherein different alleles of the gene encode proteins that differ with respect to one or more characteristics that allow the proteins to be distinguished. For example, proteins encoded by different alleles may exhibit, e.g., differences in electrophoretic mobility, differential binding to a target protein or ligand, differential binding by antibodies or other compounds (e.g., aptamers) capable of selectively binding to one or more variants of the protein, etc. For example, certain C3 variants described above exhibit “fast” or “slow” migration in a gel and can be distinguished on that basis. Protein sequencing may also be used, e.g., sequencing by mass spectrometry or Edman degradation.

In some embodiments, a complement system biomarker comprises a marker of the level of complement activation capacity or the level of complement activation in a subject. Complement activation may be measured using, e.g., a suitable assay such as a functional assay based on hemolysis (e.g., lysis of sheep or chicken red blood cells); deposition or capture of complement activation products (e.g., C3a, C3b, iC3b, C5a, MAC),etc. Pathway-specific complement activation capacity may be assessed using, e.g., appropriate stimuli and assay conditions (e.g., presence or absence of calcium ions in the assay composition) to activate one or more than one of the pathways. For example, antibody (e.g., IgM or immune complexe) can be used to activate the classical pathway; lipopolysaccharide (LPS) can be used to activate the alternative pathway, mannan can be used to activate the mannose-binding lectin portion of the lectin pathway, etc. In some embodiments, the total classical complement activity in a sample is measured using a CH50 test using antibody-sensitized sheep or chicken erythrocytes as the activator of the classical complement pathway and various dilutions of the test sample to determine the amount required to give 50% lysis. The percent hemolysis can be determined spectrophotometrically. The higher the dilution of the sample that can still achieve 50% lysis (i.e., the more diluted the sample), the greater complement activation capacity. In some embodiments, an ELISA-based assay is used. In some embodiments, complement activation is assessed based on iC3b levels, e.g., substantially as described in PCT/US2010/035871 (WO2010135717) (see Examples). In some embodiments, complement activation is assessed based on C3b levels, substantially as described in PCT/US2008/001483 (WO/2008/097525) Examples 1 and 2, respectively. In some embodiments, complement activation via the classical pathway is assessed using the MicroVue CH50 Eq EIA Kit (classical pathway), MicroVue Bb Plus EIA Kit (alternative pathway), MicroVue iC3b EIA Kit, or MicroVue C3a Plus EIA Kit (all from Quidel Corp.). In some embodiments, the amount of a complement activation product is normalized to the amount of intact C3 present in the sample prior to exposure to a complement activation stimulus. In some embodiments, complement activity, complement activation, or complement activation capacity, or the effect of a complement inhibitor, may be measured as described in any of the following US Patent App. Pub. Nos.: US20100166862, US20120141457, US 20120315266. In some embodiments, complement component C3 and the activation fragment C3d are measured in serum samples (e.g., as described in Smailhodzic D., et al., Ophthalmology, 119: 339-346.). The C3d/C3 ratio is calculated as a measure of C3 activation.

In some embodiments, a complement system biomarker level is measured in a sample obtained from a subject. In some embodiments a sample comprises a body fluid, e.g., blood, BAL fluid, sputum, nasal secretion, urine, etc. In some embodiments a sample comprises a tissue sample, which may be obtained from a cancer. In some embodiments a level is compared with a reference value. In some embodiments a reference value may be a normal value (e.g., a value within a normal range, e.g., an upper limit of a normal range). In some embodiments, if a measured value deviates significantly from a reference value or shows a trend towards increased deviation from a reference value in a manner indicative of increased complement activation, the subject may be considered to be an appropriate candidate for treatment with a complement inhibitor. A “normal range” may be a range that encompasses at least 95% of healthy individuals. In some embodiments a reference value or reference range may be a value or range associated with a disease in which complement activation plays a role, e.g., a value or range typically found in subjects suffering from such a disease in an untreated state. In such embodiments, a subject in whom the value is near or within the reference value or range may be considered to be an appropriate candidate for treatment with a complement inhibitor. In some embodiments a normal or disease-associated range may depend at least in part on demographic factors such as age, gender, etc., and can be adjusted accordingly. An appropriate reference value or range may be established empirically for different disorders and/or different biomarkers and/or, in some embodiments, for individual subjects.

In some embodiments, in vivo assessment of a complement system biomarker is envisioned. For example, in some embodiments a detectably labeled agent that binds to a product of complement activation administered to a subject. A suitable imaging method is used to visualize the agent in vivo. In some embodiments, for example, an image is obtained of the lungs, skin, or other location that may be affected by a complement-mediated disorder. In some embodiments in vivo detection allows assessment of the immunological microenvironment in a tumor, tissue, or organ of interest. In some embodiments a detectable label comprises a fluorescent, radioactive, ultrasound, or magnetically detectable moiety. In some embodiments an imaging method comprises magnetic resonance imaging, ultrasound imaging, optical imaging (e.g., fluorescence imaging or bioluminescence imaging), or nuclear imaging. In some embodiments a fluorescent moiety comprises a near-infrared or infrared fluorescent moiety (emitting in the near-infrared or infrared region of the spectrum). In some embodiments an imaging method comprises positron emission tomography (PET), and single photon emission computed tomography (SPECT). In some embodiments a detectable label is attached to an agent that binds directly to a target to be detected. In some embodiments a detectable label is associated with or incorporated into or comprises particles, which in some embodiments have at their surface an agent that binds directly to a target to be detected. PCT/US2013/055394 describes certain in vivo methods of detecting complement activation.

In some embodiments any of the biomarker assessment and/or treatment selection methods may be performed at least in part by one or more computers and/or may be stored in a database on a non-transitory computer medium. In some embodiments any of the biomarker assessment and/or treatment selection methods may be embodied or stored at least in part on a computer-readable medium having computer-executable instructions thereon. In some embodiments a computer-readable medium comprises any non-transitory and/or tangible computer-readable medium.

In some aspects, provided herein are kits comprising one or more reagents useful for performing an assay of a complement system biomarker. A kit may contain one or more probes and/or one or more primers of use in a genotyping assay described herein (e.g., a probe or primer that hybridizes selectively to one allele but not the other or permits allele-specific primer extension) or may contain an antibody or other binding agent that specifically binds to a particular variant of a complement protein (e.g., C3, CFH) or to one or more particular complement cleavage product(s) or intact complement proteins. The kit may contain one or more enzymes useful for performing a genotyping assay, one or more positive and/or negative controls, sample collection device(s), sample preparation reagents, reaction buffers. The kit may contain instructions for using it to perform an assay of a complement system biomarker. In some embodiments the kit may contain information indicating how the results are to be interpreted in terms of what they indicate regarding whether a subject is an appropriate candidate for treatment with an immune checkpoint inhibitor, whether a subject is an appropriate candidate for treatment with an immune checkpoint inhibitor and a complement inhibitor, whether a subject is likely to respond to or benefit from treatment with an immune checkpoint inhibitor, or other aspects of classifying subjects or predicting likelihood of treatment response or therapeutic benefit. In some embodiments a kit has been cleared or approved by a government regulatory agency responsible for the regulation of diagnostic tests. In some embodiments a kit has been cleared or approved as an in vitro diagnostic device or test. In some embodiments the kit contains a label specifying that it is an in vitro diagnostic device or test of use to help a health care professional determine whether the benefit to a patient from administering an immune checkpoint inhibitor (or an immune checkpoint inhibitor and a complement inhibitor) will outweigh any potential serious side effects or risks and/or to identify patients who are most likely to benefit from an immune checkpoint inhibitor (or an immune checkpoint inhibitor and a complement inhibitor). Such a kit, or the assay to be performed using the kit, may be referred to as a “companion diagnostic”.

### VI. Multifunctional Immune Checkpoint Inhibitors/Complement Inhibitors

In some embodiments, described herein are agents comprising a first moiety comprising a complement inhibitor and a second moiety that binds to a tumor antigen (TA). The second moiety may serve as a targeting moiety that targets the agent to a tumor. The agent may be used in any of the methods or composition described herein that pertain to complement inhibitors for treating a subject with cancer in combination with an immune checkpoint inhibitor. The complement inhibitor may comprise any of the complement inhibitors described herein or a functionally active portion thereof. Without limitation, in some embodiments the complement inhibitor comprises a compstatin analog or an antibody that binds to C3, C5, CFB, or CFD. A tumor antigen may be any antigenic substance produced by cancer cells or stromal cells of a tumor (e.g., cancer-associated fibroblasts or cells in tumor-associated vasculature). A TA may be a molecule (or portion thereof) that is expressed at increased levels by cancer cells or tumor stromal cells as compared with many or most non-tumor cells. A TA may at least in part exposed at the surface of cancer cells or tumor stromal cells or may be secreted. Tumor antigens may include, e.g., proteins, glycoproteins, lipids, or glycolipids that (i) are normally produced in very small quantities and are expressed in larger quantities by tumor cells, (ii) are normally produced only in certain stages of development, (iii) have a structure (e.g., sequence or post-translational modification(s)) that is modified relative to its normal structure due to mutation in tumor cells; or (iv) are normally sequestered from the immune system in healthy subjects. A TA may be an expression product of a mutated gene (e.g., an oncogene, a mutated tumor suppressor gene, or any gene that is mutated in a cancer cell), an overexpressed or aberrantly expressed protein, an antigen encoded by an oncogenic virus (e.g., HBV; HCV; herpesvirus family members such as EBV, HHV-8, CMV; papilloma virus, etc.), or an oncofetal antigen. Oncofetal antigens are normally produced during embryonic or fetal development and then largely or completely disappear but may be expressed by tumors. Examples are alphafetoprotein (AFP, found e.g., in germ cell tumors and hepatocellular carcinoma) and carcinoembryonic antigen (CEA, found, e.g., in bowel cancers and sometimes lung or breast cancer). One of ordinary skill in the art will be aware of numerous TAs and of tumor types in which such TAs are frequently found. Examples of tumor antigens include those in the following list, in which examples of various types of tumor in which the TA is found are indicated in parentheses following the name of each TA: tyrosinase (melanoma); CA-125 (ovarian cancer); MUC-1 (breast cancer, ovarian cancer); HER-2/neu (breast cancer); melanoma-associated antigen (MAGE, malignant melanoma); melanoma antigen recognized by T cells 1 (MART-1/melan-A, malignant melanoma); prostatic acid phosphatase (PAP, prostate cancer), Wilms tumor 1 protein (WT1, malignant mesothelioma, leukemias, and other solid tumors); CO17-1A (colon cancer); GD2 (a disialoganglioside expressed on tumors of neuroectodermal origin, including human neuroblastoma and melanoma); epithelial cell adhesion molecule (Epcam; epithelial tumors); cancer/testis (CT) antigens (e.g., NY-ESO-1 and LAGE-1); human telomerase reverse transcriptase (hTERT); NKG2D ligands such as MICA, MICB, or ULBP1-6; mesothelin (a glycosylphosphatidylinositol (GPI) anchored cell surface protein that is highly expressed in a number of cancers such as mesothelioma, ovarian cancer, pancreatic cancer, also found in lung adenocarcinoma, uterine serous carcinoma, cholangiocarcinoma, and squamous cell carcinoma); glypicans, e.g., glypican 3 (hepatocellular carcinoma, colorectal cancer, ovarian clear cell carcinoma, and melanoma), glypican 1 (certain breast cancers and pancreatic cancer (e.g., pancreatic ductal adenocarcinoma)); chondroitin sulfate proteoglycan-4 (melanoma, breast cancer, head and neck squamous cell carcinoma, mesothelioma, glioblastoma, clear cell renal carcinoma, and sarcomas), components of the modified subendothelial tumor extracellular matrix that may be secreted by tumor cells or tumor-associated cells (e.g., various splice isoforms of fibronectin a that contain extracellular domain B (EDB) and of tenascin-C that contain domain Al or D). In some embodiments the targeting moiety may bind to a protein that is found on normal B cells, plasma cells, or other immune system cells, such as CD19, CD20, or CD22. These proteins may be useful as targets in subjects with hematologic malignancies involving such cells, such as B cell lymphomas, Hodgkins lymphoma, and anaplastic large cell lymphoma.

Without wishing to be bound by any theory, a targeted complement inhibitor may more selectively inhibit complement activation in the tumor and/or in the vicinity of the tumor as compared with elsewhere in the body than would the same complement inhibitor without the targeting moiety. Targeting may permit use of a lower dose, may avoid systemic complement inhibition, and/or may result in a higher local concentration of the complement inhibitor as compared with in the absence of the targeting moiety.

In general, any type of binding agent may be used as the targeting moiety. For example, an antibody, a non-antibody engineered binding protein such as an affibody or adnectin, or a nucleic acid aptamer may be used. In some embodiments an scFv or single domain antibody may be used. The antibody may be human or humanized in certain embodiments. If the TA is a receptor, the targeting moiety may be ligand for the receptor, and vice versa. The TA may be selected based on the type of tumor for treatment of which the agent is to be administered (i.e., a TA commonly found in tumors of that type may be selected). In some embodiments, a subject's tumor may be analyzed to determine which TAs it expresses, and a TA expressed by the tumor is selected as a target molecule. One or ordinary skill in the art will be aware of, or can readily generate, suitable binding agents that can be used as targeting moieties. All different combinations of complement inhibitor and tumor antigen are encompassed by the present disclosure and should be considered to be expressly disclosed.

In general, any suitable method for preparing conjugates or fusion proteins may be used to generate a targeted complement inhibitor. In certain embodiments any of the methods of preparing conjugates described above, in Hermanson, supra, and/or in PCT/US2012/054180 (published as WO/2013/036778); PCT/US2012/037648 (published as WO/2012/155107); U.S. Ser. No. 14/116,591; PCT/US2013/070424 (published as WO/2014/078734); PCT/US2013/070417 (published as WO/2014/078731) may be used. The moiety that binds to a tumor antigen and the moiety comprising a complement inhibitor may be directly conjugated or fused to each other or may be joined by a linking moiety. It will be understood that modifications may be made to either or both of the agents in order to attach them to each other or to a linking moiety, such as addition of a moiety comprising a reactive functional group, and the linking portion (e.g., comprising one or more covalent bonds) created by the linkage.

In some embodiments an immune checkpoint inhibitor may be conjugated to or fused to a moiety that binds to a TA, as described above for complement inhibitors. In some embodiments the immune checkpoint inhibitor may comprise any of the immune checkpoint inhibitors described herein or a functionally active portion thereof, and the complement inhibitor may comprise any of the complement inhibitors described herein or a functionally active portion thereof. Without limitation, in some embodiments the immune checkpoint inhibitor comprises a binding agent, e.g., an antibody, that binds to CTLA4, PD1, PD-L1, PD-L2, LAG3, TIM3, BTLA, A_{2A}R, A_{2B}R, or a KIR. Without wishing to be bound by any theory, a targeted immune checkpoint inhibitor may more selectively inhibit an immune checkpoint pathway in the tumor and/or in the vicinity of the tumor as compared with elsewhere in the body than would the same immune checkpoint inhibitor without the targeting moiety. Targeting may permit the use of a lower dose, may reduce the potential for side effects, and/or may result in a higher local concentration of the immune checkpoint inhibitor as compared with in the absence of the targeting moiety. In general, any type of binding agent may be used as a targeting moiety, as described above. All different combinations of immune checkpoint inhibitor and tumor antigen are encompassed by the present disclosure and should be considered to be expressly disclosed.

In some aspects, described herein are bispecific agents comprising a first moiety comprising an immune checkpoint inhibitor and a second moiety comprising a complement inhibitor. In some embodiments the immune checkpoint inhibitor may comprise any of the immune checkpoint inhibitors described herein or a functionally active portion thereof, and the complement inhibitor may comprise any of the complement inhibitors described herein or a functionally active portion thereof. Without limitation, in some embodiments the immune checkpoint inhibitor comprises a binding agent, e.g., an antibody, that binds to CTLA4, PD1, PD-L1, PD-L2, LAG3, TIM3, BTLA, A_{2A}R, A_{2B}R, or a MR. Without limitation, in some embodiments the complement inhibitor comprises a compstatin analog or an antibody that binds to C3, C5, CFB, or CFD. In some embodiments the immune checkpoint inhibitor comprises a binding agent, e.g., an antibody, that binds to CTLA4, PD1, PD-L1, PD-L2, LAG3, TIM3, BTLA, A_{2A}R, A2_{B}R, or a KIR, and the complement inhibitor comprises a compstatin analog or an antibody that binds to C3, C5, CFB, or CFD. All different combinations of complement inhibitor and immune checkpoint inhibitor are encompassed by the present disclosure and should be considered to be expressly disclosed.

The two moieties may be directly linked to each other covalently or noncovalently or may be linked to a third moiety that links them together. Multifunctional agents comprising three or more functional domains that bind to different molecular targets (trispecific agents, tetraspecific agents) are also envisioned. For example, trispecific agents comprising two immune checkpoint inhibitors (e.g., a PD1 pathway inhibitor and a CTLA4 pathway inhibitor) and a complement inhibitor, or comprising two complement inhibitors and an immune checkpoint inhibitor, are envisioned. In some embodiments a multifunctional agent may comprise a moiety that binds to a tumor antigen.

In general, any suitable method for preparing multivalent, e.g., bivalent, trivalent, or tetravalent antibodies or conjugates may be used to generate the multifunctional agents. In some embodiments the agent is a bispecific antibody that binds to two different proteins, wherein the first protein is an immune checkpoint protein and the second protein is a complement component, e.g., C3, C5, CFD, CFB. In some embodiments the agent comprises a polypeptide chain comprising two V_{H} and two V_{L} regions linked in tandem. In some embodiments, the agent is a diabody, triabody, or tetrabody. In some embodiments, the multispecific, e.g., bispecific agent, comprises two or more chemically linked Fabs, scFvs, single domain antibodies. In some embodiments, multifunctional agents may be made using methods that make use of a suitable linker, such as those described herein. In some embodiments a bifunctional linker comprising two reactive functional groups may be used. It will be understood that suitable modifications may be made to an immune checkpoint inhibitor or complement inhibitor in order to attach the agents to each other or to a linking moiety, such as addition of a moiety comprising a reactive functional group, and the linking portion (e.g., comprising one or more covalent bonds) created by the linkage. Such modifications are within the scope of the multifunctional agents.

A bifunctional agent or multifunctional agent may be used for treating a patient in need of treatment for cancer, an infection, or any other condition in which administration of an immune checkpoint inhibitor may be of use. In some embodiments a multifunctional agent may be used instead of administering an immune checkpoint inhibitor and a complement inhibitor separately. In some embodiments a multifunctional agent may target the complement inhibitor to sites where an immune checkpoint protein to which the immune checkpoint inhibitor binds is expressed.

In some embodiments, any of the multifunctional, e.g., bifunctional, agents, described herein may be tested in one or more suitable assays to quantify its ability to bind to a TA (or to a cell that expresses a TA in vitro or in vivo), to inhibit complement activation, to modulate immune system function (e.g., to enhance an immune response to a tumor or infection). Examples of suitable assays are described herein. In some embodiments multiple agents are generated and screened, e.g., to identify those that are particularly effective, e.g., for use to treat particular cancer types or infections.

### VII. Assays of Immune System Cellular Function

In some embodiments the functional state of immune system cells (e.g., helper T cells, cytotoxic T cells, NK cells, etc.) may be measured. For example, in some embodiments an agent e.g., an immune checkpoint inhibitor, may be characterized by measuring the effect of the agent on one or more biological activities of immune system cells. In some embodiments a combination of an immune checkpoint inhibitor and a complement inhibitor on immune system cells may be characterized by measuring the effect of the combination of agents on one or more biological activities of immune system cells. In some embodiments the effect of a complement inhibitor on immune system cells that have been previously exposed to an immune checkpoint inhibitor may be measured. Those of ordinary skill in the art arc aware of suitable assays that may be used to measure immune system cell function and/or to measure the effect of an agent on immune responses. For example, a functional assay, such as cytokine secretion or proliferation in response to an appropriate stimulus. The cytokine may be interferon gamma, tumor necrosis factor alpha, or interleukin-2, may be used. In some embodiments the cell is a cytotoxic T cell or an NK cell, and the functional assay measures production or release of cytotoxic substances such as perforin, granzyme B, granulysin and/or cytotoxicity towards target cells. Cytotoxicity towards target cells may be assessed using, e.g., chromium release assays or other assays of membrane integrity such as MTT assay, induction of caspase activity, etc. In some embodiments the cell is a helper T cell, and function may be assayed by cytokine production, ability to promote the differentiation or function of other immune cell subsets, etc. In the case of a Treg, functional state may be assessed by ability of the cells to produce and/or secrete immunosuppressive cytokines or inhibit effector T cells in a functional assay. In some embodiments the functional state may be inferred from gene expression profile, cell surface marker expression, or other phenotypic characteristics. Those of ordinary skill in the art will be aware of appropriate methods of measuring synthesis and/or release of cytokines or cytotoxic substances, measuring gene expression profile, measuring cell surface marker expression, and performing functional assays. For example, synthesis or release of cytokines or cytotoxic substances may be detected using reporter assays (for measuring transcription driven by regulatory regions such as a promoter or enhancer of a gene that encodes a cytokine or cytotoxic protein), ELISA assays, and the like.

In some embodiments the ability of an agent, e.g., CTLA4 pathway inhibitor or other immune checkpoint inhibitor, to modulate (e.g., to enhance) T cell activation may be assessed by measuring one or more biological activities that occur during or as a result of T cell activation, such as T cell proliferation or cytokine production. In some embodiments the ability of an agent, e.g., a PD1 pathway inhibitor or other immune checkpoint inhibitor, to modulate (e.g., to enhance) T-cell responses and cytokine production may be assessed using in vitro assays such as the mixed lymphocyte reaction (measuring proliferation of lymphocytes in an in vitro culture challenged with MHC-incompatible cells), superantigen or cytomegalovirus stimulation assays, and others known in the art. In some embodiments an assay may be performed in vitro using isolated cells (e.g., primary cells or cells from a cell line) that are contacted with an appropriate stimulus (which may include co-stimulation), in vitro. In some embodiments an appropriate stimulus for measuring T cell activation or the effect of an agent on T cell activation may, for example, comprise anti-CD3 and anti-CD28 antibodies. In some embodiments an appropriate stimulus may comprise a cognate antigen presented by MHC (e.g., on an APC or target cell). In some embodiments the effect of one or more agents, e.g., an immune checkpoint inhibitor or a combination of an immune checkpoint inhibitor and a complement inhibitor, may be measured by contacting the cells with the agent(s) and the stimulus and measuring one or more biological of the cell (e.g., cytokine production or release, production or release of cytotoxic substances, killing of target cells, etc.).

In some embodiments, immune system cells may be isolated from a subject and their functional state may be measured ex vivo. Cells may, for example, comprise peripheral blood cells (e.g., peripheral blood mononuclear cells) from a healthy subject or from a subject suffering from a cancer or infection. In some embodiments the cells comprise tumor-infiltrating immune system cells (e.g., tumor-infiltrating lymphocytes), e.g., immune system cells (e.g., lymphocytes) isolated from lymph nodes invaded by metastatic cancer or from tumors removed by surgery, etc Immune system cells of different types or subsets, different differentiation states, and/or different functional states may be isolated and/or quantified based, e.g., on cell surface marker expression, using methods such as flow cytometry or fluorescence activated cell sorting, as known in the art. Suitable cell surface markers are known in the art, and antibodies that may be used to stain cells that display such markers are available. Antigen-specific cells specific for a particular antigen may be isolated using MHC tetramers comprising appropriate antigen. Methods of culturing immune system cells and methods of expanding them ex vivo are known in the art. In some embodiments the subject is a human subject.

In some embodiments the ability of an agent (e.g., an immune checkpoint inhibitor) or combination of agents (e.g., an immune checkpoint inhibitor and a complement inhibitor) to modulate, e.g., to enhance, an immune response may be assessed in a suitable animal model for, e.g., cancer or an infection. The assay may measure the ability of the animal to control or eliminate the cancer or infection. In some embodiments immune system cells are isolated from the non-human animal and any of the above-mentioned assays are performed. Those of ordinary skill in the art are aware of numerous non-human animal model systems for cancer and infections. Cancer models include, e.g., animals into which tumor cells are may be introduced at an orthotopic or non-orthotopic location, transgenic animals harboring activated oncogenes, etc. For example, B16-F10 murine melanoma cells may be used as an animal model for cancer, e.g., melanoma. In some embodiments tumor cells are introduced subcutaneously, under the renal capsule, or into the bloodstream. Animal models for infectious disease may be generated by infecting a non-human animal with an infectious agent that is either the pathogen of interest, a related pathogen capable of infecting the animal host (and, in some embodiments, capable of causing disease in the host), or a

In some embodiments an assay may be performed using cells isolated from a subject to whom an agent (e.g., an immune checkpoint inhibitor) or combination of agents (e.g., an immune checkpoint inhibitor and a complement inhibitor) has been administered, e.g., a subject suffering from cancer or an infection. In some embodiments any such assay may be used to measure the ability of a complement inhibitor to restore or enhance immune cell responsiveness to an immune checkpoint inhibitor. In some embodiments the complement inhibitor may be contacted with immune system cells that have become dysfunctional (e.g., exhausted, anergic) despite exposure to the immune checkpoint inhibitor. The ability of the complement inhibitor to restore cellular function may be measured. In some embodiments the complement inhibitor may be contacted with cells or administered to a subject in combination with an immune checkpoint inhibitor and the resulting immune response compared to a suitable control value measured in a comparable biological system that has not been exposed to the complement inhibitor.

In some embodiments, any of the above assays may be used to identify combinations of immune checkpoint inhibitor and complement inhibitor that are particularly effective across cancer types or in one or more particular cancer types.

### VIII. Pharmaceutical Compositions, Kits, Doses, and Administration

Suitable preparations, e.g., substantially pure preparations of an immune checkpoint inhibitor, complement inhibitor, multifunctional agent (or other active agent) may be combined with pharmaceutically acceptable carriers or vehicles, etc., to produce an appropriate pharmaceutical composition. The term “pharmaceutically acceptable carrier or vehicle” refers to a non-toxic carrier or vehicle that does not destroy the pharmacological activity of the compound with which it is formulated. One of skill in the art will understand that a carrier or vehicle is “non-toxic” if it is compatible with administration to a subject in an amount appropriate to deliver the compound without causing undue toxicity. Pharmaceutically acceptable carriers or vehicles that may be used include, but are not limited to, water, physiological saline, Ringer's solution, sodium acetate or potassium acetate solution, 5% dextrose, and the like. The composition may include other components as appropriate for the formulation desired, e.g., as discussed herein. Supplementary active compounds, e.g., compounds independently useful for treating a subject suffering from cancer or an infection, can also be incorporated into the compositions. The complement inhibitor may be any complement inhibitor, e.g., any of the complement inhibitors described herein, in various embodiments. Without limitation, in some embodiments the complement inhibitor comprises an antibody that binds to C3, CFB, CFD, C5, or a compstatin analog. The immune checkpoint inhibitor may be any immune checkpoint inhibitor, e.g., any of the immune checkpoint inhibitors described herein, in various embodiments. Without limitation, in some embodiments the immune checkpoint inhibitor comprises an antibody or other agent that binds to CTLA4, PD1, PD-L1, or PD-L2 or a soluble receptor portion that binds to PD-L1 or PD-L2. All genera, species, and combinations of genera and species of immune checkpoint inhibitor (e.g., any of the specific immune checkpoint inhibitors mentioned herein) and complement inhibitor (e.g., any of the specific complement inhibitors mentioned herein) are expressly encompassed and disclosed. Where reference is made herein to a government agency responsible for regulating pharmaceutical agents, the government agency may be, e.g., the FDA, EMA, or any other government agency having similar responsibilities, e.g., in a particular jurisdiction.

In some aspects, described herein is a pharmaceutically acceptable immune checkpoint inhibitor or pharmaceutically acceptable composition comprising a immune checkpoint inhibitor, packaged together with a package insert (label) approved by a government agency responsible for regulating pharmaceutical agents, e.g., the FDA or EMA, wherein the label includes use of the immune checkpoint inhibitor in combination with a complement inhibitor and/or wherein the package insert states that the immune checkpoint inhibitor or composition comprising an immune checkpoint inhibitor is approved for treatment of subjects who have been determined, based on an assay of a complement system biomarker, (e.g., a complement system biomarker described herein), to be appropriate candidates for treatment with the immune checkpoint inhibitor. In some embodiments, the package insert states particular patient and/or disease characteristics or criteria that define a patient population or disease category for treatment of which the immune checkpoint inhibitor or composition has been approved for use. In some embodiments, the package insert states particular patient and/or disease characteristics or criteria that define a patient population for treatment of which the immune checkpoint inhibitor or composition has been approved for use, wherein patients in the population have an increased or decreased likelihood of response, relative to patients not in the population. In some embodiments the disease category includes one or more types of cancer and/or one or more types of infection. In some embodiments, the package insert specifies a particular assay and/or a particular assay reagents or assay kit suitable for assaying a complement system biomarker and determining, based on results of the assay, whether a patient is within a patient population for treatment of which the immune checkpoint inhibitor or composition has been approved and/or suitable for determining whether the patient is in a population that has an increased or decreased likelihood of responding. In some embodiments, the label indicates particular assay results and/or patient characteristics indicative that a patient is or is not in the patient population.

In some embodiments the assay is a genotyping assay of a polymorphism or mutation in or near a complement-related gene. In some embodiments the patient characteristic(s) comprise having one or more particular alleles of a complement-related gene. In some embodiments the patient characteristic(s) comprise having a particular genetic variant at a polymorphic site in or near a complement-related gene, wherein the presence of that variant is associated with increased or decreased likelihood of response to treatment with an immune checkpoint inhibitor. In some embodiments the label states that if the patient is in a population having an increased likelihood of responding to treatment with an immune checkpoint inhibitor, the patient may acceptably be treated with an immune checkpoint inhibitor as monotherapy and/or states that the patient may acceptably be treated with an immune checkpoint inhibitor as a component of a multi-agent treatment regimen that does not include a complement inhibitor. In some embodiments the label states that if the patient is in a population having a decreased likelihood of responding to treatment with an immune checkpoint inhibitor, (i) the patient may acceptably be treated with an immune checkpoint inhibitor and a complement inhibitor, (ii) the patient may not benefit from treatment with an immune checkpoint inhibitor as monotherapy, and/or (iii) the patient may not benefit from treatment with an immune checkpoint inhibitor in the absence of a complement inhibitor. The label may specify one or more suitable complement inhibitors for combination therapy with the immune checkpoint inhibitor.

In some embodiments, the package insert states that the immune checkpoint inhibitor or composition comprising an immune checkpoint inhibitor is approved for treatment of subjects who have been determined, based on an assay of a complement system biomarker, to be appropriate candidates for treatment with the immune checkpoint inhibitor. In some embodiments, the package insert specifies that the immune checkpoint inhibitor or composition comprising an immune checkpoint inhibitor may be or should be administered in combination with a complement inhibitor. In some embodiments the package insert includes directions to treat or describing how to treat a subject who has a disorder of interest, e.g., cancer or a chronic infection, by using a combination of the immune checkpoint inhibitor and a complement inhibitor. In some embodiments the package insert states that the immune checkpoint inhibitor or composition comprising an immune checkpoint inhibitor is approved for use in combination with a complement inhibitor for treatment of subjects who have been determined, based on an assay of a complement system biomarker, to be appropriate candidates for treatment with the combination of an immune checkpoint inhibitor and complement inhibitor.

In some embodiments, described herein is a pharmaceutically acceptable complement inhibitor or a pharmaceutically acceptable composition comprising a complement inhibitor, packaged together with a package insert (label) approved by a government agency responsible for regulating pharmaceutical agents, e.g., the U.S. Food & Drug Administration, wherein the label includes use of the complement inhibitor in combination with an immune checkpoint inhibitor. In some embodiments, the package insert states particular patient and/or disease characteristics or criteria that define a patient population or disease category for treatment of which the complement inhibitor or composition has been approved for use. The label may specify one or more immune checkpoint inhibitors suitable for use in combination with the complement inhibitor. In some embodiments, the package insert or label includes directions to treat or describing how to treat a subject who has a disorder of interest, e.g., cancer or a chronic infection, by using a combination of the complement inhibitor and an immune checkpoint inhibitor. The label may specify one or more suitable immune checkpoint inhibitors. In some embodiments the package insert states that the complement inhibitor or composition comprising a complement inhibitor is approved for use in combination with an immune checkpoint inhibitor, for treatment of subjects who have been determined, based on an assay of a complement system biomarker, to be appropriate candidates for such treatment.

In some embodiments, described herein is a pharmaceutical pack or kit comprising a complement inhibitor and an immune checkpoint inhibitor, or a pharmaceutical composition comprising an immune checkpoint inhibitor and a complement inhibitor. The relative amount of each agent may be selected as appropriate for treatment of the disorder. The pack or kit may contain a package insert or label with directions to treat or describing how to treat a subject who has a disorder of interest, e.g., cancer or a chronic infection, by using a combination of the complement inhibitor and an immune checkpoint inhibitor. In some embodiments the package insert states that the combination is approved for use for treatment of subjects who have been determined, based on an assay of a complement system biomarker, to be appropriate candidates for treatment with the combination of an immune checkpoint inhibitor and complement inhibitor.

The complement system biomarker or assay specified in a package insert or label, in any of the aspects described herein, may be any complement system biomarker or assay described herein, such as an assay of the genotype of the subject with respect to a polymorphism in or near a complement-related gene.

In general, a therapeutic agent, e.g., an immune checkpoint inhibitor, complement inhibitor, or other therapeutic agent, or pharmaceutical composition comprising a therapeutic agent, may be administered using any suitable route of administration. For example, the agent or composition may be administered intravenously, intramuscularly, subcutaneously, intraarterially, by the respiratory route, intraperitoneally, topically, etc. In some embodiments of any of the methods, an immune checkpoint inhibitor, a complement inhibitor, or both, is/are administered intravenously. In some embodiments of any of the methods, an immune checkpoint inhibitor, a complement inhibitor, or both, is/are administered subcutaneously. In some embodiments of any of the methods, an immune checkpoint inhibitor is administered intravenously, and a complement inhibitor is administered subcutaneously, or vice versa.

In some embodiments, local administration to a tissue or organ affected by a disorder, e.g., cancer or a chronic infection, may be used. For example, intratumoral administration (e.g., intratumoral injection or infusion) may be used in some embodiments. In some embodiments an agent is administered into a blood vessel that supplies or is located at least in part within an organ or tissue in which a tumor is located. In some embodiments an immune checkpoint inhibitor, a complement inhibitor, or both, may be administered intratumorally. In some embodiments local administration comprises intrathecal administration for administration to the central nervous system.

It will be understood that “treatment” or “administration” encompasses, in various embodiments, directly administering a compound or composition to a subject, instructing a third party to administer a compound or composition to a subject, prescribing or suggesting a compound or composition to a subject (e.g., for self-administration), self-administration, and, as appropriate, other means of making a compound or composition available to a subject.

Pharmaceutical compositions suitable for injectable use (e.g., intravenous administration, subcutaneous or intramuscular administration) typically include sterile aqueous solutions (where water soluble) or dispersions and sterile powders for the extemporaneous preparation of sterile injectable solutions or dispersion. Injection encompasses bolus injection, intermittent or continuous infusion, e.g., using an infusion pump, etc. Sterile solutions can be prepared by incorporating the compound in the required amount in an appropriate solvent, optionally with one or a combination of ingredients such as buffers such as acetates, citrates, lactates or phosphates; agents for the adjustment of tonicity such as sodium chloride or dextrose; antibacterial agents such as benzyl alcohol or methyl parabens; antioxidants such as ascorbic acid, glutathione, or sodium bisulfite; chelating agents such as ethylenediaminetetraacetic acid; and other suitable ingredients etc., as desired, followed by filter-based sterilization. One of ordinary skill in the art will be aware of numerous physiologically acceptable compounds that may be included in a pharmaceutical composition. Other useful compounds include, for example, carbohydrates, such as glucose, sucrose, lactose; dextrans; amino acids such as glycine; polyols such as mannitol. These compounds may, for example, serve as bulking agents and/or stabilizers, e.g., in a powder and/or when part of the manufacture or storage process involves lyophilization. Surfactant(s) such as Tween-80, Pluronic-F108/F68, deoxycholic acid, phosphatidylcholine, etc., may be included in a composition, e.g., to increase solubility or to provide microemulsion to deliver hydrophobic drugs. pH can be adjusted with acids or bases, such as hydrochloric acid or sodium hydroxide, if desired. The parenteral preparation can be enclosed in ampoules, disposable syringes or infusion bags or multiple dose vials made of glass or plastic. Preferably solutions for injection are sterile and acceptably free of endotoxin.

Generally, dispersions are prepared by incorporating the active compound into a sterile vehicle which contains a basic dispersion medium and appropriate other ingredients from those enumerated above. In the case of sterile powders for the preparation of sterile injectable solutions, methods of preparation can include vacuum drying and freeze-drying which yields a powder of the active ingredient plus any additional desired ingredient, e.g., from a previously sterile-filtered solution thereof.

For administration by the respiratory route (inhalation), an agent may be delivered in the form of an aerosol spray from a pressured container or dispenser which contains a suitable propellant. A metered dose inhaler (MDI), dry powder inhaler, or nebulizer may be used. The aerosol may comprise liquid and/or dry particles (e.g., dry powders, large porous particles, etc.). Suitable aqueous vehicles useful in various embodiments include water or saline, optionally including an alcohol. In some embodiments the composition comprises a surfactant suitable for introduction into the lung. Other excipients suitable for pulmonary administration can be used. Respiratory administration may be used, e.g., in treating lung cancer, e.g., primary lung cancer or metastasis to the lung from a primary cancer elsewhere, or an infection in the lung.

A variety of different devices are available for respiratory administration. Nebulizers are devices that transform solutions or suspensions of medications into aerosols that are suitable for deposition in the lower airway. Nebulizer types include jet nebulizers, ultrasonic wave nebulizers, and vibrating mesh nebulizers. A partial list of available vibrating mesh nebulizers includes eFlow (Pari), i-Neb (Respironics), MicroAir (Omron), IH50 Nebulizer (Beurer), and Aeroneb® (Aerogen). A Respimat® Soft Mist™ Inhaler (Boeringer Ingelheim) may be used. A metered dose inhaler (MDI) is a handheld aerosol device that uses a propellant to deliver the therapeutic agent. MDIs include a pressurized metal canister that contains pharmacological agent in suspension or solution, propellant, surfactant (typically), and metering valve. Chloroflourocarbons (CFCs) had been widely used as propellants but have been largely replaced by hydrofluorocarbons (HFCs, also known as hydrofluoroalkanes (HFA)) such as HFC-134a and HFC-227ea. Carbon dioxide and nitrogen are other alternatives. A dry powder inhaler (DPI) is a breath-actuated device that delivers the drug in the form of particles contained in a capsule or blister that is punctured prior to use and typically does not employ a propellant. Examples of DPIs currently used to deliver medications for treating asthma and/or COPD include, e.g., Diskus, Aerolizer, HandiHaler, Twisthaler, Flexhaler. Such devices may be used to deliver an immune checkpoint inhibitor, complement inhibitor, or both, in various embodiments. Other exemplary DPI devices that may be used in various embodiments include 3M™ Taper and 3M Conix™, TAIFUN® (AKELA Pharma), Acu-Breathe™ (Respirics).

Inhalation accessory devices (IADs) generally fall into 2 categories: spacers and holding chambers. Spacers and holding chambers extend the mouthpiece of the inhaler and direct the mist of medication toward the mouth, reducing medication lost into the air. Using a spacer with an MDI can help reduce the amount of drug that sticks to the back of the throat, improving direction and deposition of medication delivered by MDIs. Valved holding chambers (VHCs) allow for a fine cloud of medication to stay in the spacer until the patient breathes it in through a one-way valve, drawing the dose of medicine into the lungs. Examples include Aerochamber and Optichamber.

Particulate compositions may be characterized on the basis of various parameters such as the fine particle fraction (FPF), the emitted dose, the average particle density, and the mass median aerodynamic diameter (MMAD). Suitable methods are known in the art, some of which are described in U.S. Pat. Nos. 6,942,868 and 7,048,908 and U.S. Publication Nos. 20020146373, 20030012742, and 20040092470. In certain embodiments aerosol particles are between approximately 0.5 μm-10 μm (MMAD), e.g., about 5 μm for respiratory delivery, though smaller or larger particles could also be used. In certain embodiments particles having a mass mean aerodynamic diameter of between 1 μm and 25 μm, e.g., between 1 μm and 10 μm, are used.

A dry particle composition containing particles smaller than about 1 mm in diameter is also referred to herein as a dry powder. A “dry” composition has a relatively low liquid content, so that the particles are readily dispersible, e.g., in a dry powder inhalation device to form an aerosol or spray. A “powder” consists largely or essentially entirely of finely dispersed solid particles that are relatively free flowing and capable of being readily dispersed in an inhalation device and subsequently inhaled by a subject, preferably so that a significant fraction of the particles can reach a desired portion of the respiratory tract. In certain embodiments large porous particles having mean geometric diameters ranging between 3 and 15 μm and tap density between 0.04 and 0.6 g/cm³ are used. See, e.g., U.S. Pat. No. 7,048,908; Edwards, D. et al, Science 276:1868-1871, 1997; and Vanbever, R., et al., Pharmaceutical Res. 16:1735-1742, 1999).

Various considerations for respiratory delivery that may be useful in certain embodiments are discussed in Bisgaard, H., et al., (eds.), Drug Delivery to the Lung, Vol. 26 in “Lung Biology in Health and Disease”, Marcel Dekker, New York, 2002. Aerosol devices are discussed, e.g., in Dolovich M B, Dhand R. Lancet. (2011) 377(9770):1032-45.

Oral administration may be used in certain embodiments. Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. A liquid composition can also be administered orally. Formulations for oral delivery may incorporate agents to improve stability within the gastrointestinal tract and/or to enhance absorption.

For topical application, an agent may be formulated in a suitable ointment containing the active component suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated as a suitable lotion or cream containing a compstatin analog suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

Systemic administration can also be by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration can be accomplished, e.g., through the use of nasal sprays or suppositories. In some embodiments, intranasal administration is used, e.g., to administer a complement inhibitor to a subject in need of treatment for nasal polyposis, chronic rhinosinusitis, or allergic rhinitis. For transdermal administration, the active compounds are typically formulated into ointments, salves, gels, or creams as generally known in the art.

The compounds can also be prepared in the form of suppositories (e.g., with conventional suppository bases such as cocoa butter and other glycerides) or retention enemas for rectal delivery.

Methods of local administration to the eye include, e.g., intraocular administration, e.g., intraocular injection, e.g., intravitreal injection, choroidal injection, transscleral injection, eyedrops or eye ointments, transretinal, subconjunctival bulbar, intravitreal injection, suprachoroidal injection, subtenon injection, scleral pocket or scleral cutdown injection.

In certain embodiments an active agent is prepared with carrier(s) that will protect the compound against rapid elimination from the body, such as a controlled release formulation, including implants and microencapsulated delivery systems. For example, a compound may be incorporated into or encapsulated in a microparticle or nanoparticle formulation. Biodegradable, biocompatible polymers can be used, such as ethylene vinyl acetate, polyanhydrides, polyglycolic acid, collagen, polyorthoesters, polyethers, polylactic acid, PLGA, etc. Liposomes or other lipid-based particles can be used as pharmaceutically acceptable carriers. These can be prepared according to methods known to those skilled in the art, for example, as described in U.S. Pat. No. 4,522,811 and/or other references listed herein. Depot formulations may be used, wherein an active agent released from the depot over time. One of ordinary skill in the art will appreciate that the materials and methods selected for preparation of a controlled release formulation, implant, etc., should be such as to retain activity of the compound. In some embodiments, a composition is free or essentially free of one or more carrier(s) whose primary or only intended purpose or effect would be to result in sustained or controlled release of an active agent, e.g., a complement inhibitor.

In some embodiments an immune checkpoint inhibitor, or an immune checkpoint inhibitor and a complement inhibitor, is/are administered to a subject in need of treatment for cancer or an infection in combination with one or more additional agents or treatment modalities useful in treating the cancer or infection. Any of a wide variety of anti-cancer agents or anti-infective agents may be used. The particular additional agent may be selected based on, e.g., the particular cancer or infection to be treated.

In some embodiments an immune checkpoint inhibitor, or an immune checkpoint inhibitor and a complement inhibitor, is/are administered in combination with one or more other anti-cancer agents or anti-cancer treatment modalities such as radiotherapy. Anti-cancer agents include a variety of different types of agents, including antibodies, polypeptides, and small molecules. Non-limiting examples of cancer chemotherapeutic agents that may be used include, e.g., alkylating and alkylating-like agents such as nitrogen mustards (e.g., bendamustine, chlorambucil, chlormethine, cyclophosphamide, ifosfamide, uramustine, and melphalan), busulfan, dacarbazine, procarbazine, temozolomide, thioTEPA, treosulfan, nitrosoureas (e.g., carmustine, fotemustine, lomustine, streptozocin); platinum agents (e.g., alkylating-like agents such as carboplatin, cisplatin, oxaliplatin, satraplatin, trinuclear platinum compounds such as BBR3464 and DH6C1); antimetabolites such as folic acids (e.g., aminopterin, methotrexate, pemetrexed, raltitrexed); purines such as cladribine, clofarabine, fludarabine, mercaptopurine, pentostatin, thioguanine; pyrimidines such as capecitabine, cytarabine, fluorouracil, floxuridine, gemcitabine; spindle poisons/mitotic inhibitors such as taxanes (e.g., docetaxel, paclitaxel), vincas (e.g., vinblastine, vincristine, vindesine, and vinorelbine), epothilones; cytotoxic/anti-tumor antibiotics such anthracyclines (e.g., daunorubicin, doxorubicin, epirubicin, idarubicin, mitoxantrone, pixantrone, and valrubicin), compounds naturally produced by various species of Streptomyces (e.g., actinomycin, bleomycin, mitomycin, plicamycin) and hydroxyurea; topoisomerase inhibitors such as camptotheca (e.g., camptothecin, topotecan, irinotecan) and podophyllums (e.g., etoposide, teniposide); monoclonal antibodies for cancer therapy such as anti-receptor tyrosine kinases (e.g., cetuximab, panitumumab, trastuzumab), anti-CD20 (e.g., rituximab, ofatumumab, and tositumomab), anti-CD19 (e.g., blinatumomab) and others for example alemtuzumab (an anti-CD52 antibody), gemtuzumab; photosensitizers such as aminolevulinic acid, methyl aminolevulinate, porfimer sodium, and verteporfin; tyrosine and/or serine/threonine kinase inhibitors, e.g., inhibitors of Abl, Kit, insulin receptor family member(s), VEGF receptor family member(s), EGF receptor family member(s), PDGF receptor family member(s), FGF receptor family member(s), mTOR, Raf kinase family, phosphatidyl inositol (PI) kinases such as PI3 kinase, PI kinase-like kinase family members, MEK, cyclin dependent kinase (CDK) family members, Aurora kinase family members (e.g., kinase inhibitors that are on the market or have shown efficacy in at least one phase III trial in tumors, such as cediranib, crizotinib, dasatinib, dabrafenib, erlotinib, gefitinib, imatinib, lapatinib, nilotinib, sorafenib, sunitinib, trametinib, vandetanib, vemurafenib), growth factor receptor antagonists; retinoids (e.g., alitretinoin and tretinoin); altretamine; amsacrine; anagrelide; arsenic trioxide; asparaginase (e.g., pegasparagase); bexarotene; proteasome inhibitors such as bortezomib or carfilzomib; denileukin diftitox; estramustine; ixabepilone; masoprocol; mitotane; testolactone; Hsp90 inhibitors; angiogenesis inhibitors, e.g., anti-vascular endothelial growth factor agents such as bevacizumab (Avastin) or VEGF receptor antagonists or soluble VEGF receptor domain (e.g., VEGF-Trap); matrix metalloproteinase inhibitors, etc.

In some embodiments an immune checkpoint inhibitor, or an immune checkpoint inhibitor and a complement inhibitor, is/are administered in combination with one or more agents useful for treating the infection. For example, an appropriate anti-viral, anti-bacterial, anti-fungal, or anti-parasite agent may be used, depending on the particular pathogen. For example, if the infectious agent is HIV, highly active antiretroviral therapy (HAART) may be administered. Examples of antiviral agents include, e.g., vidarabine, acyclovir, gancyclovir, valgancyclovir, nucleoside-analog reverse transcriptase inhibitors (NRT1) such as AZT (Zidovudine), ddI (Didanosine), ddC (Zalcitabine), d4T (Stavudine), or 3TC (Lamivudine), non-nucleoside reverse transcriptase inhibitors (NNRTI) such as nevirapine or delavirdine, protease inhibitors such as saquinavir, ritonavir, indinavir, nelfinavir; ribavirin, sofosbuvir, etc. Examples of antibacterial agents include, e.g., penicillins, cephalosporins, polymyxins, rifamycins, lipiarmycins, quinolones, sulfonamides, macrolides, lincosamides, tetracyclines, aminoglycosides, cyclic lipopeptides, glycylcyclines, oxazolidinones, and lipiarmycins. Examples of antifungal agents include, e.g., polyene antifungals such as amphotericin, azole antifungals (e.g., imidazoles, triazoles) that, e.g., inhibit the enzyme lanosterol 14 a-demethylase, echinocandins such as anidulafungin, caspofungin, micafungin. Examples of anti-malarial agents include, e.g., quinines, chloroquine, amiodaquine, pyrimethamine, artemesin and derivatives, doxycicine, among others. One of ordinary skill in the art will be aware of additional appropriate agents.

In some embodiments an anti-cancer agent or anti-pathogen agent comprises a second immunostimulatory agent. Certain aspects of the present disclosure provide methods comprising administering an immune checkpoint inhibitor, a second immunostimulatory agent, and a complement inhibitor to a subject in need thereof, e.g., a subject in need of treatment for a cancer or an infection. In some embodiments the second immunostimulatory agent stimulates or augments an endogenous immune response by a mechanism distinct from inhibiting an immune checkpoint pathway. In some embodiments the second immunostimulatory agent comprises a cytokine, e.g., interleukin-2, interferon-a, or GM-CSF. In some embodiments interferon-a may be used, e.g., in the treatment of hairy-cell leukaemia, AIDS-related Kaposi's sarcoma, follicular lymphoma, chronic myeloid leukaemia and malignant melanoma. In some embodiments interleukin-2 may be used, e.g., in the treatment of malignant melanoma and renal cell carcinoma. GM-CSF functions as a white blood cell growth factor that stimulates the production of granulocytes and monocytes. Monocytes exit the circulation and migrate into tissues, where they develop into macrophages and dendritic cells. GM-CSF, GM-CSF secreting cells (e.g., cells that have been genetically engineered to produce GM-CSF), or vectors (e.g., viruses) that encode GM-CSF may be used to enhance immune responses in cancer of any type.

In some embodiments a second immunostimulatory agent comprises an agent, e.g., an antibody, polypeptide, or small molecule, that acts as an agonist of an immunostimulatory receptor, e.g., a costimulatory receptor such as CD40, tumor necrosis factor receptor superfamily member 4 (TNFRSF4, also known as OX40), TNFRSF18 (also known as GITR), tumor necrosis factor receptor superfamily member 9 (TNFRSF9, also known as 4-1BB and CD137), or inducible costimulator (ICOS).

In some embodiments a second immunostimulatory agent comprises an oncolytic virus. Oncolytic virotherapy uses replication competent viruses to destroy cancers (Russell, S J, et al., Nat Biotechnol. (2012); 30(7):658-70). Some viruses have a natural preference for cancer cells, whereas others can be adapted or engineered to make them cancer-specific. Viruses may also or alternatively be engineered to enhance efficacy, such as by engineering them to comprise a gene encoding an immunostimulatory cytokine such as GM-CSF or to reduce pathogenicity, such as by deleting or disabling a gene involved in replication of the virus in non-dividing cells. In addition to direct killing of cancer cells, administration (e.g., intratumoral or intravenous injection) of replicative oncolytic viruses such as herpes simplex, pox-, parvo-, or adenoviruses stimulates the immune system. Talimogene laherparepvec, HSV-1 [strain JS1] ICP34.5-/ICP47-/hGM-CSF, (previously known as OncoVEX^{GM CSF}), is an example of an oncolytic virus. It comprises an immune-enhanced HSV-1 that selectively replicates in solid tumors and comprises a gene that encodes GM-CSF. (Lui et al., Gene Therapy, 10:292-303, 2003; U.S. Pat. Nos. 7,223,593 and 7,537,924.

In some embodiments a second immunostimulatory agent comprises a cancer vaccine. Examples of cancer vaccines include, e.g., Sipuleucel-T (Provenge), Oncophage, GVAX, oncolytic viruses, tarmogens, and other agents that directly or indirectly provide tumor antigens to generate or stimulate an immune response to a tumor or comprising immune system cells that have been stimulated ex vivo with tumor antigens. In some embodiments a second immunostimulatory agent comprises a bacterium (e.g., an attenuated bacterium) that has been genetically engineered to express a tumor antigen. For example, Listeria monocytogenes (Lm) is a gram-positive bacterium that selectively infects antigen-presenting cells wherein it is able to efficiently deliver tumor antigens to both the MHC Class I and II antigen presentation pathways for activation of tumor-targeting CTL-mediated immunity. Lm is able to induce therapeutic immunity against a wide-array of TAs and specifically infect and kill tumor cells directly.

In some embodiments a second immunostimulatory agent comprises a, toll-like receptor (TLR) agonist, e.g., a TLR3 agonist such as poly-IC (e.g., in the for mof pICLC (poly-IC condensed with poly-L-lysine and carboxymethylcellulose); a TLR4 agonist such as 3-O-desacyl-4′-monophosphoryl lipid A (MPL); a TLR7 agonist such as imiquimod or resiquimod (a mixed TLR7/TLR8 agonist); or a TLR9 agonist such as CpG oligodeoxynucleotides. In some embodiments a TLR agonist may be used as a cancer vaccine adjuvant. One or more TLR agonists may be administered as part of a cancer vaccine or as a separate composition. Of course other adjuvants known in the art can be used.

In some embodiments a second immunostimulatory agent comprises a STING agonist.

In some embodiments a second immunostimulatory agent comprises cell-based immunotherapy. Cell-based tumor immunotherapy holds great promise, but its efficacy may be hindered by immune checkpoint pathways. Certain aspects of the present disclosure encompass administration of cell-based immunotherapy together with an immune checkpoint inhibitor. In some embodiments the subject is determined to be a suitable candidate for treatment with an immune checkpoint inhibitor based on an assay of a complement system biomarker. Certain aspects of the present disclosure encompass administration of cell-based immunotherapy together with an immune checkpoint inhibitor and a complement inhibitor. In some embodiments the subject is determined to be a suitable candidate for treatment with an immune checkpoint inhibitor and a complement inhibitor based on an assay of a complement system biomarker.

Cell-based immunotherapy encompasses adoptive immunotherapy, e.g., which comprises the introduction of immune system cells into a subject, e.g., by infusion into the circulatory system. The introduced cells mount an immune response and/or augment an endogenous immune response against a cancer (e.g., cancer cells, cancer-associated cells) or pathogen. In some embodiments the introduced cells comprise peripheral blood mononuclear cells, lymphocytes, NK cells, dendritic cells (DCs), or a combination thereof. In some embodiments the lymphocytes comprise T cells (CD4+ T cells, CD8+ T cells), B cells, or both. In some embodiments the cells comprise cytotoxic cells, e.g., CD8+cytotoxic T cells or NK cells, that attack the tumor. In some embodiments the cells comprise tumor infiltrating lymphocytes (TILs). In some embodiments the cells comprise cytokine-induced killer (CIK) cells. In some embodiments the cells comprise immature cells that differentiate into effector cells of any of the afore-mentioned types. The immune system cells may be autologous or may be obtained from a donor (e.g., an immunocompatible donor). The cells may be expanded and/or activated ex vivo as known in the art (e.g., using IL-2 and anti-CD3 antibodies). The cells may be administered in combination with one or more cytokines.

In some embodiments cells may be genetically engineered ex vivo prior to introducing them into the subject. In some embodiments the immune system cells naturally or as a result of genetic engineering express an antigen receptor that specifically binds to a tumor antigen. In some embodiments the cells may additionally or alternately be genetically engineered to express a TA, an immunostimulatory cytokine, or both. The tumor antigen (TA) may be any TA. Various TAs that may be used are mentioned above. In some embodiments the antigen receptor is a chimeric antigen receptor (CAR). CARs comprise an antigen recognition domain fused to a transmembrane domain and an endomain, wherein the endodomain transmits a stimulatory signal to the cell. A cell, e.g., a T cell, may be genetically engineered to express a CAR. Suitable genetic engineering methods may be used. In some embodiments gene transfer by a retroviral vector may be used. Typically the endomain comprises at least one immunoreceptor tyrosine-based activation motif (ITAM), e.g., 2 or 3 ITAMs, though more may be used. In some embodiments the CD3-zeta or CD3-epsilon chain transmembrane (TM) domain and endodomain may be used. In some embodiments the CD28 TM domain may be used. The antigen recognition domain may be any suitable polypeptide that binds to a desired target, e.g., a tumor antigen, and can be expressed as part of a fusion protein at the cell surface, so that the antigen recognition domain can bind to a target antigen, e.g., on a cancer cell. In some embodiments an scFv may be used. In some embodiments a non-antibody polypeptide may be used such as an affibody, adnectin, anticalin, or the like. “Second-generation” CARs add intracellular signaling domains from various costimulatory protein receptors (e.g., CD28, 41BB, ICOS) to the cytoplasmic tail of the CAR to provide additional signals to the T cell. “Third-generation” CARs combine multiple signaling domains, such as CD3z-CD28-41BB or CD3z-CD28-OX40. See, e.g., Sadelain M, et al., Cancer Discov. 2013; 3(4):388-98, for further discussion of CARs and their design.

Dendritic cells (DCs) can be stimulated to activate a cytotoxic response towards an antigen. Dendritic cells may be pulsed ex vivo with an antigen or genetically engineered to express a tumor antigen (TA), e.g., by transfection with a vector, e.g., a viral vector, encoding the TA. Upon infusion into the patient the DCs present tumor antigens to lymphocytes. This initiates a cytotoxic response against cells expressing tumour antigens (against which the adaptive response has now been primed). An example of this approach is the product Sipuleucel-T.

It will be understood that the classification of anti-cancer agents herein is non-limiting. A number of anti-cancer agents have multiple activities or mechanisms of action and could be classified in multiple categories or classes or have additional mechanisms of action or targets. It will also be understood that additional antibodies, small molecules, or engineered binding proteins that bind to the molecular targets of antibodies or small molecules mentioned above may be used, e.g., for the same purposes.

The anti-cancer agent may be selected based on the particular cancer type being treated and/or the stage or grade or other clinical or pathological criteria, e.g., the agent may have demonstrated efficacy in treating cancers of that type, may be approved for treating cancers of that type, may have demonstrated efficacy or been approved for treating patients with cancers having particular stage or grade or meeting other clinical or pathological criteria. In some embodiments a molecularly targeted anti-cancer agent may be used. A “molecularly targeted anti-cancer agent” refers to an agent that acts on, and typically inhibits, a particular molecular target, e.g., a gene product of an oncogene, that is overexpressed, activated, or otherwise dysregulated in cancer cells and contributes to causing or sustaining one or more aspects of the malignant phenotype. In some embodiments the molecular target may be a substrate of an oncogenic protein. Typically, a molecularly targeted anti-cancer agent binds to its molecular target. In some embodiments the molecular target is a kinase, e.g., a tyrosine kinase or a serine/threonine kinase, such as those above mentioned. It will be understood that a molecularly targeted anti-cancer agent may not be completely specific for its target. In some embodiments it may act on a group of structurally related targets. For example, many kinase inhibitors inhibit multiple kinases. In some embodiments a molecularly targeted therapy acts on a mutant form of the target. It may or may not act on the normal form of the target. For example, V600 is an oncogenic mutation in BRAF and is found in some melanomas. Dabrafenib and vemurafenib are approved by the FDA for the treatment of unresectable or metastatic BRAF V600-positive melanoma. In some embodiments a BRAF inhibitor is used in combination with an immune checkpoint inhibitor for treatment of melanoma in a subject who has been determined to be an appropriate candidate for treatment with an immune checkpoint inhibitor based on an assay of a complement system biomarker. In some embodiments a BRAF inhibitor is used in combination with an immune checkpoint inhibitor and a complement inhibitor for treatment of melanoma in a subject who has been determined to be an appropriate candidate for treatment with an immune checkpoint inhibitor and a complement inhibitor based on an assay of a complement system biomarker. In some embodiments, treatment with a MEK inhibitor, e.g., trametinib, is useful in combination with a BRAF inhibitor and may be included in any treatment regimen that includes a BRAF inhibitor. In some embodiments the immune checkpoint inhibitor is a CTLA4 inhibitor, e.g., ipilimumab.

When two or more agents (e.g., compounds or compositions) are used or administered “in combination” with each other, also referred to as “combination therapy”, co-administration”, they may be given at the same time, within overlapping time periods, or sequentially (e.g., separated by up to 2-4 weeks, 4-6 weeks, 6-8 weeks, or 8-12 weeks, in time), at least once, in various embodiments. The agents may be administered in the same composition or can be administered separately but sufficiently closely in time so as to provide the desired therapeutic effect. A person of ordinary skill in the art would readily determine appropriate timing, sequence, and dosages of administration for particular agents and compositions described herein. It will be understood that any sequence may be applied repeatedly, and that different time intervals may be used over a course of treatment. There may be one or more cycles of administration of a first agent, followed by one or more cycles of administration of a second agent, and such cycles may be repeated one or more times. Agents administered in combination may be administered via the same route or different routes in various embodiments. They may be administered in either order in various embodiments. In some embodiments an agent is administered at least once between two doses of another agent. In some embodiments an agent is administered at least once between every second, third, or fourth dose of another agent. In some embodiments, agents are administered within 4, 8, 12, 24, 48, 72, or 96 hours of each other at least once. In some embodiments, agents are administered within 4, 8, 12, 24, 48, 72, or 96 hours of each other multiple times. In some embodiments, a first agent is administered prior to or after administration of the second agent, e.g., sufficiently close in time that the two agents are present at useful levels within the body at least once. In some embodiments, the agents are administered sufficiently close together in time such that no more than 50%, 75%, or 90% of the earlier administered agent has been metabolized to inactive metabolites or eliminated, e.g., excreted, from the body, at the time the second agent is administered.

It will be appreciated that a compound may be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Also, pharmaceutically-acceptable salts can be prepared as alkaline metal or alkaline earth salts, such as sodium, potassium or calcium salts, if appropriate depending on the identity of the active agent.

It will be understood that the pharmaceutically acceptable carriers, compounds, and preparation methods mentioned herein are exemplary and non-limiting. See, e.g., Remington: The Science and Practice of Pharmacy. 21st Edition. Philadelphia, Pa. Lippincott Williams & Wilkins, 2005, for additional discussion of pharmaceutically acceptable compounds and methods of preparing pharmaceutical compositions of various types.

An agent can be used or administered to a subject in an effective amount. In some embodiments, an “effective amount” of an active agent, e.g., a complement inhibitor or immune checkpoint inhibitor refers to an amount of the active agent sufficient to elicit one or more biological effect(s) of interest in, for example, a subject to whom the active agent (or composition) is administered. As will be appreciated by those of ordinary skill in the art, the absolute amount of a particular agent that is effective may vary depending on such factors as the biological endpoint, the particular active agent, the target tissue, etc. Those of ordinary skill in the art will further understand that an “effective amount” may be administered in a single dose, or may be achieved by administration of multiple doses. In some embodiments an effective amount of a complement inhibitor or a composition comprising a complement inhibitor may be an amount sufficient to reduce the level of complement activation or complement activation capacity in a subject by a selected amount (e.g., a selected percentage). For example, in some embodiments the level of complement activation or complement activation capacity is reduced by at least 10%, 20%, 30%, 40%, 50%, 60%, 70%, 80%, 90%, 95%, or more, as determined using a suitable assay. In some embodiments the level is determined in a biological sample obtained from the subject, e.g., blood (or plasma or serum) or other body fluid or a tissue sample (e.g., a sample of tumor tissue). In some embodiments an effective amount of an agent or composition may be an amount sufficient to achieve one or more of the following in a cancer patient: a complete response, a partial response, or stable disease as determined by objective criteria; an improvement in symptoms, an increase in progression-free survival, an increase in overall survival. In some embodiments an effective amount of an agent or composition may be an amount that suppresses (e.g., eliminates) replication of a pathogen in a subject suffering from an infection, renders a subject free of the infectious agent, renders the subject non-infectious, results in an improvement in symptoms of infection, decreases mortality due to the infection, and/or an increases overall survival. Those of ordinary skill in the art will appreciate that a wide variety of assays are available for detecting and/or quantifying pathogens and/or their replication. These may include, e.g., measuring pathogen-specific nucleic acids or proteins, culturing the pathogen from a biological sample obtained from a subject, measuring a surrogate marker. In some embodiments the biological effect is enhancement of the efficacy of a second agent. An effective amount of a composition, e.g., a pharmaceutical composition, contains an effective amount of one or more agents. If the composition comprises multiple active agents, the agents may be present in an amount such that the overall composition is effective.

In general, appropriate doses of immune checkpoint inhibitor, complement inhibitor, or other active agent depend at least in part upon the potency of the immune checkpoint inhibitor, complement inhibitor or other active agent, route of administration, etc. In general, dose ranges that are effective and well tolerated can be selected by one of ordinary skill in the art. Such doses can be determined using clinical trials as known in the art. In some embodiments, an animal model is used, for example, to help guide selection of a dose, dose range, or formulation for testing in humans, to assess one or more biological effect(s), etc. Those of ordinary skill in the art will understand that an effective amount for treating a disorder may be established based on the effect of the agent in a population of subjects to whom it is administered, e.g., in a clinical trial, and that the agent may not produce the desired therapeutic effect in every subject. Those of ordinary skill in the art will also understand that certain agents are typically used in combination with other therapies, and that an “effective amount” of such an agent for treating a disorder may be an amount such that the therapeutic effect of interest is produced by the combination of the agent and the other therapies, also used at their effective amounts. Optionally, a dose may be tailored to the particular recipient, for example, through administration of increasing doses until a preselected desired response is achieved, such as a preselected desired degree of complement inhibition in the case of a complement inhibitor. If desired, the specific dose level for any particular subject may be selected based at least in part upon a variety of factors including the activity of the specific compound employed, the particular condition being treated and/or its severity, the age, body weight, general health, route of administration, any concurrent medication, and/or the status of the complement system as determined based on one or more complement biomarkers, which may be measured in one or more samples obtained from the subject. In some embodiments an effective amount or dose of an agent, e.g., an immune checkpoint inhibitor or a complement inhibitor, ranges from about 0.001 to 500 mg/kg body weight, e.g., about 0.01 to 100 mg/kg body weight, e.g., about 0.1 to 50 mg/kg body about 0.1 to 20 mg/kg body weight, e.g., about 1 to 10 mg/kg. In some embodiments an effective amount may be between 1 mg and 10,000 mg, e.g., between 1 mg and 10 mg, between 10 mg and 100 mg, between 100 mg and 1000 mg, between 1000 mg and 2000 mg.

One of ordinary skill in the art will appreciate that methods of determining appropriate dosages of immune checkpoint inhibitors to administer to a cancer patient, either alone or in combination with one or more other agents (e.g., a complement inhibitor, a second immunostimulatory agent, etc.), may be determined by performing dose-response and toxicity studies using methods that are well known in the art. In certain embodiments, an immune checkpoint inhibitor that has been approved for administration to human subjects may be administered at a dose indicated in the approved label (prescribing instructions). In certain embodiments, an immune checkpoint inhibitor may be administered at about 0.1 mg/kg-20 mg/kg, or the maximum tolerated dose. In some embodiments the agent may be administered about every 2-6 weeks, e.g., about every 2 weeks, about every 3 weeks, about every 4 weeks, or about every 6 weeks. In some embodiments an immune checkpoint inhibitor antibody may be administered by an escalating dosage regimen including administering a first dosage at about 3 mg/kg, a second dosage at about 5 mg/kg, and a third dosage at about 9 mg/kg. Another escalating dosage regimen may include administering a first dosage of immune checkpoint inhibitor antibody about 3 mg/kg, a second dosage of about 3 mg/kg, a third dosage of about 5 mg/kg, a fourth dosage of about 5 mg/kg, and a fifth dosage of about 9 mg/kg. Exemplary dosages of immune checkpoint inhibitor antibodies include 3 mg/kg ipilimumab administered every three weeks for four doses; 10 mg/kg ipilimumab every three weeks for eight cycles; 10 mg/kg every three weeks for four cycles then every 12 weeks; 10 mg/kg MK-3475 every two or every three weeks; 2 mg/kg MK-3475 every three weeks; 15 mg/kg tremilimumab every three months; 0.1, 0.3, 1, 2, 3 or 10 mg/kg nivolumab every two weeks for up to 96 weeks (or longer); 0.3, 1, 3, or 10 mg/kg BMS-936559 every two weeks for up to 96 weeks (or longer) (Kyi C. & Postow, M A, FEBS Lett. (2014) 588(2):368-76; Callahan, M K & Wolchok, J D (2013) J Leukoc Biol 94:41-53); pembrolizumab at doses of 2 mg/kg and 10 mg/kg every 3 weeks (Hamid, O., N Engl J Med. 2013; 369(2):134-44); BMS-936559 at doses of 1 mg/kg, 3 mg/kg, or 10 mg/kg every 2 weeks (Brahmer, J R, et al., N Engl J Med 2012; 366:2455-65); pidilizumab at 3 mg/kg intravenously every 4 weeks (Westin, J R, et al., Lancet Oncol. 2014 January; 15(1):69-77).

In certain embodiments, a complement inhibitor that has been approved for administration to human subjects may be administered at a dose indicated in the approved label (prescribing instructions). In certain embodiments, a complement inhibitor, e.g., a compstatin analog or an antibody, may be administered at about 0.1 mg/kg-20 mg/kg, or the maximum tolerated dose. In some embodiments the agent may be administered about every 2-6 weeks, e.g., about every 2 weeks, about every 3 weeks, about every 4 weeks, or about every 6 weeks. In certain embodiments a complement inhibitor, e.g., a compstatin analog or an antibody, may be administered at a dose of about 0.05 mg/kg/day to about 2 mg/kg/day. In certain embodiments a complement inhibitor, e.g., a compstatin analog or an antibody, may be administered at a dose of about 2 mg/kg/day to about 5 mg/kg/day. It will be understood that the dose need not be administered daily, e.g., the afore-mentioned amount may be administered at less frequent intervals such that an average amount of 0.05 mg/kg/day to about 2 mg/kg/day is administered. In some embodiments, eculizumab may be administered at 600 mg-1200 mg, e.g., 900 mg-1200 mg, every 2 weeks.

In some embodiments, a method comprises administering at least 5, 10, 15, 20, or 25 doses of a complement inhibitor a subject. In some embodiments, treatment with a complement inhibitor is continued over a period of at least 1, 2, 3, 6, 9, 12 months, or more, e.g., 1-2 years, 2-5 years, or more, e.g., as long as treatment with an immune checkpoint inhibitor is continued. In some embodiments, between 1 and 5 doses or between 5 and 10 doses of a complement inhibitor may be sufficient to inhibit or overcome resistance to treatment with an immune checkpoint inhibitor. The subject may be retreated with a complement inhibitor if resistance re-emerges. In some embodiments a complement inhibitor may be administered so as to inhibit complement in the cancer or in the subject's blood for a period of at least 1, 2, 3, 4, 6, or 8 weeks. In some embodiments complement is inhibited for a limited period of time, e.g., up to about 1, 2, 3, 4, 6, or 8 weeks. In some embodiments a single course of complement inhibitor is administered. In some embodiments two or more courses of therapy may be administered, with the complement system being allowed to return to its usual state in between courses.

It will be appreciated that the dosing interval and dosage amount may vary over the course of therapy. In some embodiments, a dosing interval or dosage amount of an immune checkpoint inhibitor, complement inhibitor, or both, for a subject may vary over time and/or may be selected at least in part based on a biomarker. For example, in some embodiments a dosing interval, dosage amount, or time for retreating a subject may be based on measurement of complement activation or complement activation capacity in the subject or in a sample obtained from the subject and/or assessment of disease activity (or a biomarker thereof) between doses.

In some embodiments, a course of treatment comprises two or more phases, e.g., an induction phase and a subsequent phase. In many embodiments, the induction phase (if used) occurs when a subject initiates therapy with a particular agent or combination of agents. The induction phase can consist of a period of time during which an agent, e.g., an immune checkpoint inhibitor, a complement inhibitor, or both, is/are administered at a higher dose and/or at more frequent intervals and/or using a different route of administration than during the subsequent phase. In some embodiments an induction phase may last for up to 1, 2, 3, 4, 5, 6, 7, 8, 10, or 12 weeks. In some embodiments a dose or dosing interval is adjusted during an induction phase. For example, in some embodiments the dosing interval may be increased over time and/or the dose may be decreased or increased over time during the induction phase. In some embodiments an escalating dose regimen may be used. In some embodiments retreatment may occur on a fixed time schedule.

Further provided herein are methods comprising instructing a patient or health care provider in the use of a composition or method described herein, where the term “instructing” in this context means providing directions for the relevant treatment, treatment regimen, complement system biomarker assay, or the like, by any means, e.g., writing. Instructing can be in the form of prescribing a course of treatment, or can be in the form of package inserts or other written material. It will be understood that such methods may be applied in the context of any composition or method described herein. Instructions may specify one or more acceptable doses, dosing intervals, routes of administration, methods of monitoring the patient, etc.

Further provided are methods comprising marketing a composition or method described herein, wherein “marketing” refers to the promotion (e.g., advertising), selling, distribution of a product (e.g., a pharmaceutical agent or composition) or other activity involving or directed to commercializing a product. It will be understood that such methods may be applied in the context of any composition or method described herein.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. It will be appreciated that the invention is in no way dependent upon particular results achieved in any specific example or with any specific embodiment. Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. For example, and without limitation, it is understood that where claims or description indicate that a residue at a particular position may be selected from a particular group of amino acids or amino acid analogs, the invention includes individual embodiments in which the residue at that position is any of the listed amino acids or amino acid analogs. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims or from the description above is introduced into another claim. For example, any claim that is dependent on another claim can be modified to include one or more elements, limitations, clauses, or descriptive terms, found in any other claim that is dependent on the same base claim. Furthermore, where the claims recite a composition, it is to be understood that methods of administering the composition according to any of the methods disclosed herein, and methods of using the composition for any of the purposes disclosed herein are included within the scope of the invention, and methods of making the composition according to any of the methods of making disclosed herein are included within the scope of the invention, unless otherwise indicated or unless it would be evident to one of ordinary skill in the art that a contradiction or inconsistency would arise. Methods of treating a subject can include a step of providing a subject in need of such treatment (e.g., a subject who has been diagnosed with the disease or is at increased risk of developing a disease), a step of diagnosing a subject as having a disease and/or a step of selecting a subject for treatment with one or more agents described herein.

Where elements are presented as lists, it is to be understood that each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects or embodiments of the invention, is/are referred to as comprising particular elements, features, etc., certain embodiments of the invention or aspects of the invention consist, or consist essentially of, such elements, features, etc.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Any embodiment, aspect, element, feature, etc., of the present invention may be explicitly excluded from the claims. For example, any immune checkpoint inhibitor, complement inhibitor, formulation, disorder, subject population or characteristic(s), dosing interval, administration route, biomarker, assay, or combination of any of the foregoing, can be explicitly excluded.

1. A method of treating a subject in need of treatment of cancer comprising administering an immune checkpoint inhibitor and a complement inhibitor to the subject.

2. (canceled)

3. The method of claim 1, wherein the subject has been determined, based on an assay of one or more complement system biomarkers, to be unlikely to respond to therapy with an immune checkpoint inhibitor.

4. (canceled)

5. The method of claim 1, wherein the subject has been previously treated for the cancer with an immune checkpoint inhibitor in the absence of combination treatment with a complement inhibitor and did not respond.

6. The method of claim 1, wherein the subject has been previously treated for the cancer with an immune checkpoint inhibitor in the absence of combination treatment with a complement inhibitor and exhibited a response followed by disease progression.

7.-8. (canceled)

9. The method of claim 1, wherein the method further comprises treating the subject with a second anti-cancer agent.

10.-15. (canceled)

16. A method of treating a cancer patient comprising steps of: (a) obtaining results of an assay of one or more a complement system biomarker in a cancer patient or in a sample obtained from a cancer patient; (b) correlating the results with the likelihood that the patient will respond to therapy with an immune checkpoint inhibitor; and (c) based on step (b) either (i) treating the cancer patient with an immune checkpoint inhibitor or (ii) treating the cancer patient with an immune checkpoint inhibitor and a complement inhibitor or (iii) treating the cancer patient with a second anti-cancer therapy in combination with or instead of an immune checkpoint inhibitor.

17. The method of claim 16, wherein results of the assay of a complement system biomarker comprises the genotype of the subject or cancer with respect to a polymorphism in or near of a complement-related gene.

18.-35. (canceled)

36. A method of treating a subject in need of treatment for cancer comprising: (a) treating the subject with an immune checkpoint inhibitor; (b) evaluating the subject one or more times after initiating treatment with the immune checkpoint inhibitor; (c) determining that the subject exhibits progressive disease; and (d) treating the subject with a complement inhibitor in combination with the same or a different immune checkpoint inhibitor.

37. The method of claim 36, wherein the method comprises treating the subject with a complement inhibitor if the subject does not respond to the immune checkpoint inhibitor within 6 months of initiating treatment or exhibits progressive disease after an initial response.

38. The method of claim 1, wherein the cancer is a melanoma, lung cancer, bladder cancer, head and neck cancer, ovarian cancer, renal cell carcinoma (RCC), prostate cancer, or hematological malignancy.

39.-40. (canceled)

41. The method of claim 1, wherein the immune checkpoint inhibitor comprises an antibody, an engineered binding protein, a soluble receptor, an aptamer, or a small molecule that binds to an immune checkpoint protein.

42. (canceled)

43. The method of claim 1, wherein the immune checkpoint inhibitor inhibits the PD1 pathway.

44. The method of claim 1, wherein the immune checkpoint inhibitor binds to PD1, PD-L1 or PD-L2.

45. The method of claim 1 of claim 44, wherein the immune checkpoint inhibitor comprises an antibody that binds to PD1 or PD-L1.

46. (canceled)

47. The method of claim 1, wherein the checkpoint inhibitor inhibits the CTLA4 pathway.

48. The method of claim 47, wherein the immune checkpoint inhibitor binds to CTLA4.

49.-51. (canceled)

52. The method of claim 1, wherein the immune checkpoint inhibitor inhibits an immune checkpoint pathway involving LAG3, TIM3, BTLA, A2AR, or A2BR.

53. The method of claim 1, wherein the complement inhibitor comprises an antibody, aptamer, peptide, polypeptide, or small molecule that binds to a complement component.

54. The method of claim 1, wherein the complement inhibitor binds to C3, C5, factor B, or factor D.

55. The method of claim 1, wherein the complement inhibitor inhibits cleavage of C3, C5, or factor B.

56.-57.

58. The method of claim 1, wherein the complement inhibitor comprises a compstatin analog whose sequence comprises any of SEQ ID NOs: 3-41.

59.-60. (canceled)

61. The method of claim 1, wherein the immune checkpoint inhibitor comprises ipilimumab, tremilimumab, nivolumab, pembrolizumab (lambrolizumab), pidilizumab, MPDL3280A (an Fc engineered anti-PD-L1), BMS-936559, MPDL3280A, MEDI4736, MSB0010718C, or any combination of these.

62. (canceled)
